SEQUENCE LISTING (1 sequence)

SEQ ID NO: 1 - DNA, 126 bp, Homo sapiens
  source 1..126: mol_type = other DNA
gtgttgtatgaaaggagagaaggttagcactccccttgacaaggatggaagaggccctcgggcctgacaacacgcatacggttaaggcattgccacctacttcgtggcatctaaccatcgtttttt

(19) United States
(12) Patent Application Publication
(10) Pub. No.: US 2024/0325429 A1
(43) Pub. Date: Oct. 3, 2024

(54) MINOR SPLICEOSOME TARGETING COMPOSITIONS AND THEIR USE IN CANCER TREATMENT

(71) Applicant: UNIVERSITÄT BERN, Bern (CH)

(72) Inventors: Anke AUGSPACH, Bern (CH); Mark RUBIN, Schliern bei Köniz (CH)

(73) Assignee: UNIVERSITÄT BERN, Bern (CH)

(21) Appl. No.: 18/691,491

(22) PCT Filed: Aug. 17, 2022
(86) PCT No.: PCT/EP2022/072931

§ 371 Date: Mar. 13, 2024

(30) Foreign Application Priority Data
Aug. 17, 2021 (EP) 21191594.7

(51) Int. Cl. A61K 31/713; A61P 35/00; C12N 15/113; C12Q 1/6886

(52) U.S. Cl. CPC A61K 31/713; A61P 35/00; C12N 15/113; C12Q 1/6886; C12Q 2600/106; C12Q 2600/158

(57) ABSTRACT

The present invention relates to a pharmaceutical nucleic acid agent capable of downregulating or inhibiting the activity of the minor spliceosome (MiS) for use in treatment or prevention of recurrence of cancer. The invention also relates to a method for the diagnosis of cancer.

This application claims the benefit of the European Patent Application EP21191594.7 filed 17 Aug. 2021, which is incorporated herein by reference.

The present invention relates to a pharmaceutical nucleic acid agent capable of downregulating or inhibiting the activity of the minor spliceosome (MiS), for use in treatment, or prevention of recurrence, of cancer. The invention also relates to a method for the diagnosis of cancer.

### BACKGROUND OF THE INVENTION

Cancer evolves continuously by modifying its transcriptome as it evades therapeutic interventions. Splicing, which is routinely leveraged, results in altered isoforms of key cancer genes involved in disease progression. Currently, little is known about MiS in cancer progression. Here the inventors explore PCa as an exemplar cancer to understand the role of MiS in disease progression.

Androgen deprivation therapy (ADT) is used to treat advanced PCa. However, for PCa resistant to ADT, second-generation potent androgen receptor signalling inhibitors (ARSi) such as enzalutamide and abiraterone are used. While initially effective, intrinsic or acquired resistance to ARSi in form of castration resistant prostate cancer (CRPC) eventually develops in this ultimately lethal disease. In CRPC resistance to ARSi is conferred by intra-tumoral heterogeneity driven by re-activation of the AR axis including AR amplifications, AR mutations, AR co-activators and ligand independent AR activations.

Another increasingly recognized mechanism in the context of prolonged ARSi treatment (AR suppression) is the trans-differentiation form adenocarcinoma (CRPC-adeno) to neuroendocrine PCa (CRPC-NE), an extremely lethal and AR-indifferent variant of PCa. Generally, CRPC-adeno and CRPC-NE are defined by expression/absence of characteristic markers such as AR and KLK3 (CRPC-adeno) or SYP and CHGA and AR absence (CRPC-NE). The expression of those markers can be quantified in so called AR- or NEPC-scores.

While CRPC-adeno and CRPC-NE share similar genomic landscapes, they have dramatically distinct transcriptomes, suggesting non-coding RNA events and RNA splicing as a potential mechanism of PCa transdifferentiation and progression. Indeed, multiple studies propose that alternative splicing of the AR transcript plays a key role in therapy resistance of CRPC-adeno. While the splicing factor SRRM4 has been identified as a crucial driver of CRPC-NE transdifferentiation. In general, non-canonical splicing has been extensively linked to prostate tumorigenesis and many PCa relevant genes display isoform switching during cancer development and progression. Yet little is known about the pathways controlling it and a unified hypothesis explaining the molecular origin of those isoforms is still lacking. Thus, understanding the mechanisms underlying aberrant splicing in PCa is essential both for predicting tumor progression and for discovering key regulators. While the role of canonical splicing in cancer has been studied extensively, the present understanding of the interplay between minor intron splicing and cancer is lacking.

Minor introns (<0.5%), which require the minor spliceosome, are found in genes with mostly major introns that are spliced by the major spliceosome. These minor intron-containing genes (MIGs) execute diverse functions in disparate molecular pathways. Despite the diverse functions, MIGs are highly enriched in the essentialome, a list of genes that are essential for survival. The essentiality of MIGs is reflected in early embryonic lethality when MiS is inhibited in mice, zebrafish, and Drosophila. Moreover, loss of U11 snRNA in the developing pallium results in aberrant splicing of MIGs that resulted in cell cycle defect and loss of rapidly dividing neural stem cells. Regarding cancer, the dysregulation of minor intron splicing has been linked to the Peutz-Jegher's syndrome and myelodysplastic syndrome which frequently proceed to gastrointestinal cancer and acute myeloid leukemia, respectively. There is further evidence that MiS components show a reliable association with an increased risk of scleroderma (U11/U12-65K protein), AML (U11-59K protein) and familial PCa (U11 snRNA). In fact, microRNA (miRNA) profiling studies of high-risk Finnish PCa families identified altered U11 snRNA expression as a risk factor to develop PCa.

Based on the above-mentioned state of the art, the objective of the present invention is to provide means and methods to treat or prevent the recurrence of cancer. This objective is attained by the subject-matter of the independent claims of the present specification, with further advantageous embodiments described in the dependent claims, examples, figures and general description of this specification.

### SUMMARY OF THE INVENTION

Here the inventors show that MiS function, which is regulated by the AR-axis, increases with (prostate) cancer disease stage and degree of differentiation. In fact, MiS component, U6atac snRNA, might serve as an additional marker for cancer diagnostics. The inventors show that siU6atac-mediated MiS inhibition is more effective at blocking PCa cell proliferation than the current state of the art combination therapy such as EZH2 inhibitor/enzalutamide. Finally, the inventors show that other MiS components can also be targeted, and that MiS inhibition also blocks proliferation of other cancer cell types. In all, this work brings to light a novel pathway, the minor spliceosome, as point of entry for therapeutics against lethal PCa and that this strategy extends to other cancer types.

A first aspect of the invention relates to a pharmaceutical nucleic acid agent capable of downregulating or inhibiting the activity of the minor spliceosome (MiS) for use in treatment or prevention of recurrence of cancer.

A second aspect of the invention relates to a method for assigning a likelihood of having or developing cancer to a patient. A high likelihood of having or developing cancer is assigned if an expression level of snRNA U6atac is 2-3.

A third aspect of the invention relates to a pharmaceutical nucleic acid agent for use according to the first aspect, wherein a high likelihood of having or developing cancer, or of having or developing a cancer of advanced, therapy resistant phenotype is assigned to the patient according the method of the second aspect.

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term snRNA U6atac in the context of the present specification relates to

(SEQ ID NO 001)
GTGTTGTATGAAAGGAGAGAAGGTTAGCACTCCCCTTGACAAGGATGGA
AGAGGCCCTCGGGCCTGACAACACGCATACGGTTAAGGCATTGCCACCT
ACTTCGTGGCATCTAACCATCGTTTTTT (ENSG00000221676).

### General Molecular Biology: Nucleic Acid Sequences, Expression

The term gene refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

The terms gene expression or expression, or alternatively the term gene product, may refer to either of, or both of, the processes—and products thereof—of generation of nucleic acids (RNA) or the generation of a peptide or polypeptide, also referred to transcription and translation, respectively, or any of the intermediate processes that regulate the processing of genetic information to yield polypeptide products. The term gene expression may also be applied to the transcription and processing of a RNA gene product, for example a regulatory RNA or a structural (e.g. ribosomal) RNA. If an expressed polynucleotide is derived from genomic DNA, expression may include splicing of the mRNA in a eukaryotic cell. Expression may be assayed both on the level of transcription and translation, in other words mRNA and/or protein product.

The term downregulating or inhibiting expression in the context of the present specification relates to the ability to reduce the number of RNA molecules inside a cell.

The term Nucleotides in the context of the present specification relates to nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). Wherever reference is made herein to a hybridizing sequence, such hybridizing sequence may be composed of any of the above nucleotides, or mixtures thereof.

The terms capable of forming a hybrid or hybridizing sequence in the context of the present specification relate to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and on the backbone chemistry.

In the context of the present specification, the term hybridizing sequence encompasses a polynucleotide sequence comprising or essentially consisting of RNA (ribonucleotides), DNA (deoxyribonucleotides), phosphothioate deoxyribonucleotides, 2′-O-methyl-modified phosphothioate ribonucleotides, LNA and/or PNA nucleotide analogues. In certain embodiments, a hybridizing sequence according to the invention comprises 8, 9, 10, 11, 12, 13, 14, 15, 16, 17, 18, 19, 20, 21, 22, 23, 24, 25, 26, 27, 28, 29 or 30 nucleotides. In certain embodiments, the hybridizing sequence comprises deoxynucleotides, phosphothioate deoxynucleotides, LNA and/or PNA nucleotides or mixtures thereof.

The term antisense oligonucleotide in the context of the present specification relates to an oligonucleotide having a sequence substantially complimentary to, and capable of hybridizing to, an RNA. Antisense action on such RNA will lead to modulation, particular inhibition or suppression of the RNA's biological effect. If the RNA is an mRNA, expression of the resulting gene product is inhibited or suppressed. Antisense oligonucleotides can consist of DNA, RNA, nucleotide analogues and/or mixtures thereof. The skilled person is aware of a variety of commercial and non-commercial sources for computation of a theoretically optimal antisense sequence to a given target. Optimization can be performed both in terms of nucleobase sequence and in terms of backbone (ribo, deoxyribo, analogue) composition. Many sources exist for delivery of the actual physical oligonucleotide, which generally is synthesized by solid state synthesis.

The term siRNA (small/short interfering RNA) in the context of the present specification relates to an RNA molecule capable of interfering with the expression (in other words: inhibiting or preventing the expression) of a gene comprising a nucleic acid sequence complementary or hybridizing to the sequence of the siRNA in a process termed RNA interference. The term siRNA is meant to encompass both single stranded siRNA and double stranded siRNA. siRNA is usually characterized by a length of 17-24 nucleotides. Double stranded siRNA can be derived from longer double stranded RNA molecules (dsRNA). According to prevailing theory, the longer dsRNA is cleaved by an endo-ribonuclease (called Dicer) to form double stranded siRNA. In a nucleoprotein complex (called RISC), the double stranded siRNA is unwound to form single stranded siRNA. RNA interference often works via binding of an siRNA molecule to the mRNA molecule having a complementary sequence, resulting in degradation of the mRNA. RNA interference is also possible by binding of an siRNA molecule to an intronic sequence of a pre-mRNA (an immature, non-spliced mRNA) within the nucleus of a cell, resulting in degradation of the pre-mRNA.

The term shRNA (small hairpin RNA) in the context of the present specification relates to an artificial RNA molecule with a tight hairpin turn that can be used to silence target gene expression via RNA interference (RNAi).

The term sgRNA (single guide RNA) in the context of the present specification relates to an RNA molecule capable of sequence-specific repression of gene expression via the CRISPR (clustered regularly interspaced short palindromic repeats) mechanism.

The term miRNA (microRNA) in the context of the present specification relates to a small non-coding RNA molecule (containing about 22 nucleotides) that functions in RNA silencing and post-transcriptional regulation of gene expression.

### Binding:

The term specific binding in the context of the present invention refers to a property of ligands that bind to their target with a certain affinity and target specificity. The affinity of such a ligand is indicated by the dissociation constant of the ligand. A specifically reactive ligand has a dissociation constant of ≥10⁻⁷ mol/L when binding to its target, but a dissociation constant at least three orders of magnitude higher in its interaction with a molecule having a globally similar chemical composition as the target, but a different three-dimensional structure.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

### DETAILED DESCRIPTION OF THE INVENTION

A first aspect of the invention relates to a pharmaceutical nucleic acid agent capable of downregulating or inhibiting the activity of the minor spliceosome (MiS) for use in treatment or prevention of recurrence of cancer.

In certain embodiments, the agent is capable of downregulating or inhibiting expression of snRNA U6atac, particularly by hybridizing to, and leading to degradation or inhibition of, SEQ ID No 001.

In certain embodiments, the agent is or encodes an antisense oligonucleotide. In certain embodiments, the agent is or encodes an siRNA.

In certain embodiments, said cancer is a lethal cancer of advanced, therapy resistant phenotype (neuroendocrine and adenocarcinomas, particularly neuroendocrine carcinomas).

In certain embodiments, said cancer is selected from glioblastoma, breast cancer, chronic myeloid leukemia (CML), bladder cancer, colon cancer, kidney cancer and prostate cancer.

In certain embodiments, said cancer is prostate cancer.

In certain embodiments, said cancer is selected from castration-resistant prostate cancer (CRPC), neuroendocrine prostate cancer (NEPC), castration-resistant neuroendocrine prostate cancer (CRPC-NE) and small cell prostate cancer. In certain embodiments, said cancer is a lethal cancer of androgen receptor negative NE type and/or androgen receptor negative and NE negative type. Advanced resistant prostate cancers do not respond to ADT (androgene deprivation therapy) and ARSi (Androgene receptor inhibitors).

In certain embodiments, said agent is administered in combination with a platinum-containing complex.

In certain embodiments, said agent is administered in combination with a platinum-containing drug selected from carboplatin, satraplatin, cisplatin, dicycloplatin, nedaplatin, oxaliplatin, picoplatin, and/or triplatin.

A second aspect of the invention relates to a method for assigning a likelihood of having or developing cancer to a patient. A high likelihood of having or developing cancer is assigned if an expression level of snRNA U6atac is 2-3. For in situ testing there are 4 possible levels 0, 1, 2, 3. 2-3 would be equivalent to moderate/strong expression.

In certain embodiments, a high likelihood of having or developing cancer is assigned if the expression level of snRNA U6atac is 2 or 3.

In certain embodiments, a high likelihood of having or developing a cancer of advanced, therapy resistant phenotype is assigned if an expression level of snRNA U6atac is 2 or 3. In certain embodiments, a high likelihood of having or developing a cancer of advanced, therapy resistant phenotype is assigned if the expression level of snRNA U6atac is 2 or 3.

The inventors have observed a strong difference, regarding U6atac expression, between 1) benign tissue and cancer 2) primary and advanced or metastatic cancer. Their conclusion is that the relationship is valid for any cancer, wherein a higher U6atac score signals a more advanced stage of cancer.

In certain embodiments of the second aspect, the cancer is selected from glioblastoma, breast cancer, chronic myeloid leukemia (CML), bladder cancer, colon cancer, kidney cancer and prostate cancer. In certain embodiments of the second aspect, the cancer is prostate cancer.

A third aspect of the invention relates to a pharmaceutical nucleic acid agent for use according to the first aspect, wherein a high likelihood of having or developing cancer, or of having or developing a cancer of advanced, therapy resistant phenotype is assigned to the patient according the method of the second aspect.

A further aspect of the invention relates to a method for treatment or prevention of recurrence of cancer in a patient, said method comprising administering a pharmaceutical nucleic acid agent according to the first aspect to a patient.

A further aspect of the invention relates to a method for treatment or prevention of recurrence of cancer in a patient, said method comprising the steps:
a. obtaining a tumour sample from said patient;
b. determining an expression level of snRNA U6atac in said sample;
c. identifying a patient with high risk of recurrence of cancer if said expression level of snRNA U6atac is 2 or 3;
d. treating said patient with high risk of recurrence of cancer with antineoplastic drug.

In certain embodiments, the patient is treated via administering a pharmaceutical nucleic acid agent according to the first aspect.

A further aspect of the invention relates to a system for performing the method according to the second aspect.

A further aspect of the invention relates to a use of an agent being able to determine the expression level of snRNA U6atac in the manufacture of a kit for the detection of cancer.

### Medical Treatment, Dosage Forms and Salts

Similarly, within the scope of the present invention is a method or treating cancer in a patient in need thereof, comprising administering to the patient a nucleic acid sequence vector according to the above description.

Similarly, a dosage form for the prevention or treatment of cancer is provided, comprising a non-agonist ligand or antisense molecule according to any of the above aspects or embodiments of the invention.

The skilled person is aware that any specifically mentioned drug compound mentioned herein may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for parenteral administration, such as subcutaneous, intravenous, intrahepatic or intramuscular injection forms. Optionally, a pharmaceutically acceptable carrier and/or excipient may be present.

### Pharmaceutical Compositions and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

The pharmaceutical composition can be formulated for enteral administration, particularly oral administration or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The pharmaceutical composition can be formulated for parenteral administration, for example by i.v. infusion, intradermal, subcutaneous or intramuscular administration.

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of a nucleic acid agent as identified herein, or its pharmaceutically acceptable salt, as specified in detail above, for use in a method of manufacture of a medicament for the treatment or prevention of cancer.

Similarly, the invention encompasses methods of treatment of a patient having been diagnosed with a disease associated with cancer. This method entails administering to the patient an effective amount of a nucleic acid agent as identified herein, or its pharmaceutically acceptable salt, as specified in detail herein.

The invention further encompasses the use of a nucleic acid agent able to detect an snRNA U6atac expression level identified herein for use in the manufacture of a kit for the detection of cancer.

Wherever alternatives for single separable features such as, for example, a target gene, nucleic acid agent type or medical indication are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### DESCRIPTION OF THE FIGURES

FIG. 1 shows that MIG expression patterns and MiS component expression correlate with PCa progression. A) Enrichment of MIGs (542) amongst proteins interacting at a distance (d)=1, d=2, d=3, d=4 and d=5 with proteins encoded by 403 cancer and 26 prostate cancer genes in a human protein-protein interaction network. Enrichment values show the log 2 fold change of the presence of MIGs in distance bins, compared to randomly sampled gene sets of equal size. Error bars indicate standard deviations. Asterisk indicates P<10E-5. B) Largest connected subnetwork of all interactions between prostate cancer genes and MIGs captured 87 genes (inset). Curve indicates the distribution of network sizes when the inventors randomly sampled sets of non-MIGs of equal size (542). C) Subnetwork of interactions between 74 MIGs and 19 prostate cancer genes (amplification of inset in 1B). D) Relative performance of MIGs and non-MIGs with respect to clustering gene expression data from nine distinct cancer types from PCAWG (Biliary-, Breast-, Colorectal-, Lung-, Ovary-, Pancreas-, Stomach-, Thymus-, and Prostate-Adenocarcinoma). Each box corresponds to a 1000-length simulation in which non-MIGs are randomly sampled from among all non-MIGs in the genome. This plot exhibits a negative relationship between the relative abundance of non-MIGs and the quality of clustering, as quantified using the Silhouette coefficient. The P-value is based on a two-sided t-test in which each sample value is taken to be the difference between the Silhouette coefficient of one of the 1000 100% non-MIG samples and the corresponding Silhouette coefficient when only MIGs are used for clustering (i.e., the Silhouette coefficient corresponding to 0% non-MIGs). E) Relative performance of MIGs and non-MIGs with respect to clustering gene expression data from different stages of prostate cancer progression. Gene expression data were taken from GTEx, TCGA, and SU2C; respectively, these datasets represent increasing stages of tumor progression (from healthy tissue in GTEx to advanced Prostate tumors in SU2C). Clustering was performed using an n_cluster value of K=3. Each box in this plot corresponds to a 1000-length simulation in which non-MIGs are randomly sampled from among all non-MIGs in the genome. This plot exhibits a negative relationship between the relative abundance of non-MIGs and the quality of clustering, as quantified using the Silhouette coefficient. The P-value is based on a two-sided t-test in which each sample value is taken to be the difference between the Silhouette coefficient of one of the 1000 100% non-MIG samples and the corresponding Silhouette coefficient when only MIGs are used for clustering (i.e., the Silhouette coefficient corresponding to 0% non-MIGs). F) Boxplots representing the distribution of RNU11 expression values (counts per million (CPM)) across primary tumor samples where RNU11 was detected. Samples (n=23) were grouped according to low (6+7) or high (8+9) Gleason score (n=23, Wilcoxon Test, p-value indicated). G) Pearson correlation analysis between RNPC3 and SOX2. Organoids are color-coded according to their transcriptomic NEPC score, whereby a score >0.4 indicates a CRPC-NE phenotype while a score<0.4 indicates a CRPC-Adeno phenotype (Beltran et al., PMID 26855148). Organoids are size-coded according to their AR-score. H) Heatmap showing RNA-seq expression (FPKM) of prostate cancer cell lines, ordered by increasing NEPC and decreasing AR score. The NEPC and AR score were calculated based on FPKM values of a set of 70 and 27 genes to estimate the likelihood of a test sample to be CRPC-NE or CRPC-adeno, respectively. 1) U6atac snRNA expression as x-fold of siScrambled normalized to the mean of GAPDH and ACTB gene transcription in different PCa cell lines (RWPE n=5, LNCaP n=7, DU145 n=7, PC3 n=3, PCc8 n=9, L-AR n=10, C4-2 n=7, 22Rv1 n=5, PM154 n=5, H660=5). Data are represented using box and whisker plots. Boxplots display values of minimum, first quartile, median, third quartile, and maximum (two-sided Mann-Whitney test, ns; not significant, p>0.05, *p<0.05, **p<0.01, ***p<0.001). Each data point represents the x-fold of LNCaP cells from a single experiment. Experiments were performed in triplicates. J) U6atac score of the TMA analysis (Wilcoxon Test, p=0.0038).

FIG. 2 shows that MiS activity is regulated by AR and is elevated in prostate cancer. A) Representative images of the validation of RNU6atac BaseScope probes in human primary, primary with metastatic potential and metastatic PCa tissue. Scale bars represent 50 μm. Quantification of the U6atac score of the whole TMA analysis shown on the right-hand side. Comparisons between groups were performed with Wilcox test (primary vs. primary (metastatic: p=0.004, primary vs. metastasis: p<2.22e-16, primary (metastatic) vs. metastasis: p=0.013). B) Normalized luminescence values of minor/major spliceosome luc-reporter plasmids in different PCa cell lines of different PCa subtypes (RWPE n=9, LNCaP n=30, DU145 n=7, PC3 n=10, C4-2 n=22, 22Rv1 n=13, VCAP n=7, PM1262 n=13, PM154 n=20, MSK16 n=7, H660=12, mean±SEM, one-way Anova; ns p>0.05, *p<0.05, **p<0.01, ***p<0.001). Each data point represents a single experiment; experiments were performed in triplicates. Abbreviations: Pri-PCa=Primary Prostate Cancer, CRPC-Ad=Castration Resistant Adeno Prostate Cancer, CRPC-NE=Castration Resistant Neuroendocrine Prostate Cancer. C) Normalized luminescence values of minor/major spliceosome luc-reporter plasmids in LNCaP and C4-2 PCa cells subjected to long-term treatment with abiraterone, enzalutamide and charcoal stripped (c/s) media (LNCaP: Abiraterone n=19, c/s n=17, Enzalutamide n=7, C4-2: Abiraterone n=24, c/s n=29, Enzalutamide n=12, mean±SEM, one-way Anova; ns p>0.05, *p<0.05, **p<0.01, ***p<0.001). Each data point represents a single experiment; experiments were performed in triplicates. D) Normalized luminescence values of minor/major spliceosome luc-reporter plasmids in LNCaP and L-AR cells treated with siAR, c/s media and c/s media+DHT (10 uM) (LNCaP n=34, L-AR n=19, L-AR+siAR n=7, L-AR c/s n=8, L-AR c/s+DHT n=13, mean±SEM, ordinary one-way Anova; ns p>0.05, *p<0.05, **p<0.01, ***p<0.001). Each data point represents a single experiment, that were performed in triplicates. Abbreviations: c/s=charcoal stripped media, DHT=Dihydrotestosterone. E) Representative 40× confocal photomicrographs of PLA signal from AR and PDCD7 or HSP90 (positive control) heteromeric formation (red puncta) and associated negative IgG rabbit control. PLA was performed using anti-PDCD7 antibody (rabbit monoclonal) and androgen receptor antibody (mouse monoclonal) primary antibody and oligonucleotide-linked PLA secondary probes [mouse, M(+) and rabbit, R(−)]. Scale bars represent 25 μm. The experiment was repeated 3 times.

FIG. 3 shows that inhibition of the MiS through U6atac siRNA effectively alters the PCa transcriptome. A) U6atac snRNA expression as x-fold of siScrambled (control) normalized to the mean of GAPDH and ACTB gene transcription in C4-2 cells (U6atac KD n=14, U6atac over. n=7) Data are represented using box and whisker plots that display values of minimum, first quartile, median, third quartile, and maximum. Statistical analysis was evaluated using two-sided unpaired t-test, ns p>0.05, *p<0.05, **p<0.01, ***p<0.001. Each data point represents a single experiment; experiments were performed in triplicates. B) Expression of CoA3 spliced and unspliced transcripts as x-fold of siScrambled normalized to the mean of GAPDH and ACTB gene transcription in C4-2 cells treated with siU6atac RNA for 48, 72 and 96 hours (n=3, two-way Anova; ns p>0.05, *p<0.05, **p<0.01, ***p<0.001). Experiments were performed in triplicates. C) Box-plot showing 10^{th}-90^{th} percentile mis-splicing index for all minor introns that show retention (431) in LNCaP, C4-2, and 22Rv1 cell lines as well as PM154 organoid after 96 h treatment with siScrambled (scr) or siU6atac. Significance determined by Kruskal-Wallis test followed by post-hoc Dunn's test: asterisk (*) denotes significance compared to each siU6atac samples' appropriate siScrambled control; plus sign (+) denotes significance compared to LNCaP siU6atac; hashtag (#) denotes significance compared to PM154 siU6atac. *p<0.05, **p<0.01, ***p<0.001. D) Bar chart showing the distribution of alternative splicing (AS) events around (from two exons upstream to two exons downstream) all minor introns in LNCaP, C4-2, and 22Rv1 cell lines and the PM154 organoid after 96 h treatment with siScrambled (scr) or siU6atac. Different colors depict different alternative splicing categories. E) Venn-diagram (not to scale) depicting the overlap of MIGs with significantly elevated minor intron retention in the LNCaP, C4-2, and 22RV1 cell lines (left) and the PM154 organoid (right) after 96 h siU6atac treatment compared to the appropriate 96 h siScrambled control. F) Venn-diagram (not to scale) depicting the overlap of MIGs with significantly elevated alternative splicing in the LNCaP, C4-2, and 22RV1 cell lines (left) and the PM154 organoid (right) after 96 h siU6atac treatment compared to the appropriate 96 h siScrambled control. G) STRING network showing association of shared (i) prostate cancer-associated MIGs with elevated minor intron retention (red) and (ii) downregulated protein coding genes (grey; downregulated MIGs in blue) in all three cell lines LNCaP, C4-2, and 22RV1 (left) and PM154 organoid (right). H) Venn-diagram (not to scale) showing the overlap of Ingenuity Pathway Analysis (IPA)-generated biological pathways for the LNCaP, C4-2, and 22RV1 (LCR) gene list (G; left) and PM154 (P) gene list (G; right). 1) Venn-diagram (not to scale) showing the overlap of Ingenuity Pathway Analysis (IPA)-generated biological networks for the LNCaP, C4-2, and 22RV1 (LCR) gene list (H; left) and PM154 (P) gene list (G; right).

FIG. 4 shows that U6atac mediated MiS inhibition effectively alters the PCa proteome in a cell-type specific manner. A) Volcano plot showing proteins most significantly increased (upper right) and decreased (upper left) in C4-2 cells treated with siU6atac (96 h), as compared to the siScrambled control (pooled data from 3 co-IP replicates). The x-axis represents log 2 fold change (FC) values, the y-axis represents −log 10 of adjusted p-values. Grey dots represent non-differentially expressed (non-DE) proteins; orange dots represent differential expressed proteins (DE), black dots represent differentially expressed MIG-encoded proteins (DE MIG) and red dots represent differentially expressed MIG-encoded proteins that transcriptomically have significantly elevated minor intron retention. B) Venn diagrams (not to scale) illustrating the overlap in proteins that are upregulated after U6atac KD in LNCaP, C4-2, 22Rv1 and PM154 cells, assessed by mass spectrometry analysis. C) Venn diagrams (not to scale) illustrating the overlap in proteins that are downregulated after U6atac KD in LNCaP, C4-2, 22Rv1 and Pm154 cells, assessed by mass spectrometry analysis. D) Immunoblot showing expression levels of selected proteins which are down- or upregulated in mass spectrometry analysis in C4-2 cell lines treated for 96 hours with siScrambled or siU6atac RNA. GAPDH was used as loading control. E) Table summarizing differential expression of selected proteins after siU6atac treatment in LNCaP (L), C4-2 (C), 22Rv1 (R) and PM154 (P) cells. F) Venn-diagrams (not to scale) illustrating the overlap of genes that show upregulation (left) or downregulation (right) transcriptionally (trans.) and by mass spec (prot.) in LNCaP, C4-2, 22Rv1, and PM154 cells. G) Quantification of flow cytometry analysis of C4-2 and PM154 cells treated with siU6atac for 72 h and 96 h, followed by staining with EdU and Hoechst (n=3, mean±SEM, ordinary two-way Anova; ns p>0.05, *p<0.05, **p<0.01, ***p<0.001). H) Psuedocolour plot showing the gates for G1/0, S and G2 selection in C4-2 cells treated for 72 h with siU6atac or siScrambled. Quantification of flow cytometry analysis of PM154 organoids treated with siU6atac for 72 h and 96 h, followed by staining with EdU and Hoechst (n=3, mean±SEM, ordinary two-way Anova; **p=0.001, ****p<0.001). 1) Psuedocolour plot showing the gates for G1/0, S and G2 selection in PM154 organoids treated for 72 h with siU6atac or siScrambled. Quantification of flow cytometry analysis of PM154 organoids treated with siU6atac for 72 h and 96 h, followed by staining with EdU and Hoechst (n=3, mean±SEM, ordinary two-way Anova; **p=0.001, ****p<0.001).

FIG. 5 shows that single cell RNAseq corroborates siU6atac-mediated cell cycle defects and reveals PCa lineage dependency on MiS function. A) UMAP representation of LNCaP cell line showing the clusters at the optimal resolution (0.2). B) Histogram of LNCaP cell line showing the contribution of each sample in each cluster. C) UMAP representation of LNCaP cell line showing the cell cycle phase of LNCaP cell line for each cell. D) Histogram of LNCaP cell line showing the percentage of cell cycle phase in each cluster. E) UMAP representation of the LNCaP cell line showing the contribution of siScrambled and siU6atac samples (on the left); the cell cycle phase of siScrambled and siU6atac samples (on the center) and cell phase of each cell (on the right). F) UMAP representation of PM154 cell line showing the contribution of siScrambled and siU6atac samples (on the left); the cell cycle phase of siScrambled and siU6atac samples (on the center) and cell phase of each cell. G) Histogram of LNCaP cell line showing the percentage of the cell cycle phase for siScrambled and siU6atac cells. The table is showing the number of cells for each cell cycle phase. The p-value was calculated using a Fisher's exact test. H) Histogram of PM154 cell line showing the percentage of the cell cycle phase for siScrambled and siU6atac cells. The table is showing the number of cells for each cell cycle phase. The p-value was calculated using a Fisher's exact test. 1) UMAP representation of LNCaP cell line showing the AR score calculated on siScrambled and siU6atac samples. Violin plots show the AR score calculated for each cell cycle phase. The p-value was calculated using a Wilcoxon test. J) UMAP of LNCaP cell line showing the EMT score calculated on siScrambled and siU6atac samples. Violin plots show the EMT score calculated for each cell cycle phase. The p-value was calculated using a Wilcoxon test.

FIG. 6 shows that U6atac and the MiS represent potential therapeutic targets in cancer. A) Cell viability in human PCa cell lines (LNCaP n=5, L-AR n=4, C4-2 n=6, 22Rv1 n=6, Pm154 n=6), human fibroblast cells (HS27 n=6) and primary mouse prostate cells (MS2514 n=3, MS2513 n=3) (mean±SEM, ordinary two-way Anova; ns p>0.05, *p<0.05, **p<0.01, ***p<0.001). Experiments were performed in triplicates. B) Growth curves of C4-2 cells stable overexpressing U6atac normalized to C4-2 cells expressing the EV plasmid or C4-2 cells treated for several time points with siU6atac normalized to C4-2 cells treated with siScrambled. Data represents pooled results from 16 (C4-2 siU6atac) and 4 (C4-2 U6atac) biologically independent experiments (mean+/−SD ordinary two-way Anova; ns p>0.05, *p<0.05, **p<0.01, ***p<0.001). C) Growth curves of LNCaP and L-AR cells treated for several time points with siU6atac. Data is normalized to the siScrambled control and represents pooled results from 4 biologically independent experiments (mean+/−SEM, ordinary two-way Anova; ns p>0.05, *p<0.05, **p<0.01, ***p<0.001). D) Cell viability in different human cancer cell lines treated for several time points with siU6atac. Data are normalized to the siScrambled control and represents pooled results from 4 biologically independent experiments (mean+/−SEM ordinary two-way Anova; ns p>0.05, *p<0.05, **p<0.01, ***p<0.001). E) Cell viability in human bladder cancer UMOC-9 cells treated with siRNA against U6atac, RNPC3 and Scrambled or with Cisplatin (1 uM). Data represents pooled results from 4 biologically independent experiments (mean+/−SD, ordinary one-way Anova; ns p>0.05, *p<0.05, **p<0.01, ***p<0.001). F) Growth curves of LNCaP (n=4), L-ENZ (n=6) and C4-2 (n=4) cells treated with siScrambled, siU6atac and siEZH2+/−Enzalutamide (10 uM). Data represents pooled results from biologically independent experiments (mean+/−SEM, ordinary two-way Anova; ns p>0.05, *p<0.05, **p<0.01, ***p<0.001). G) Scatter blot representing RNAseq results from patient derived PCa organoids (big dots) superimposing clinical data from the SU2C dataset (small dots). Y-axis represents the AR-score, x-axis represents the NEPC score. H) Brightfield microcopy of MSK8 cells 8 days after siRNA treatment against U6atac. Cells treated with Scrambled siRNA are shown as control. Scale bars (red): 50 μm. Bar blot summarizes cell counts at day 8 (n=3, paired t-test, *p=0.0307). 1) Cell viability in patient derived PCa organoids. Data is normalized to the scrambled control and represents pooled results from three biologically independent experiments (mean+/−SEM, two-way Anova; ns p>0.05, *p<0.05, **p<0.01, ***p<0.001).

FIG. 7 shows that U6atac expression is increased in cancer. A) Representative images of RNU6atac BaseScope probe expression in human ovary, small cell lung cancer (SCLC), (ADC), skin, uterus and prostate benign (n=15) and tumor tissue (n=33). Scale bars represent 20 μm. Lower panel shows the U6atac score of the TMA analysis (Wilcoxon Test, p=0.0038). B) U6atac intensity in the different samples (Wilcoxon Test, stomach: p=8.78E-9, tyroid follicular carcinoma: p=0.63, tyroid poorly differentiated carcinoma: p=6.26E-6).

FIG. 8 shows that U6atac expression is increased in metastatic PCa. U6atac score across all samples tested (Wilcoxon Test, primary vs. primary with metastatic potential: p=0.0004, primary vs. metastasis: p<2.2E-16, primary with metastatic potential vs. metastasis: p=0.013).

FIG. 9 shows that MiS component expression and MiS activity increases with PCa progression. A) Cell viability measured using CellTiter-GLO in human PCa cell lines treated with 20 uM enzalutamide for 48 hours. Data was normalized to the untreated WT control (ctr.) (LNCaP n=6, L-AR n=3, L-rENZ n=6, C4-2 n=3, 22Rv1 n=4, PM154 n=6, PC3 n=3, PC3Pro4 n=3, mean±SEM, ordinary one-way Anova; ns p>0.05, *p<0.05). Experiments were performed in triplicates. B) Normalized luminescence values of minor/major spliceosome luc-reporter plasmids in PC3 cells (n=10) and its subline PC3PPro4 (n=4) (upper row) and LNCaP cells (n=37), its AR overexpressing subline L-AR (n=19) and enzalutamide resistant subline L-rENZ (n=7, mean±SD, ordinary one-way Anova; ns p>0.05, ***p, ****p<0.0001). Each data point represents a single experiment, experiments were performed in triplicates. C) hSCN4A and p120 reporter plasmid splice index in PC3, PC3Pro4, LNCaP and L-rENZ cells (n=3, two-sided unpaired t-test, ****p<0.0001). Splice index was calculated as the ratio of hSCN4A and p120 spliced vs. unspliced expression values of cells transfected with the hSCN4A, and p120 minigene reporter plasmids for 72 h. Expression values were normalized to the mean of GAPDH and ACTB gene transcription and calculated as x-fold of siScrambled (siScr.).

FIG. 10 shows that siU6atac RNA decreases MiS activity. A) U6atac and p120 spliced and unspliced expression as x-fold of siScrambled (siScr.) normalized to the mean of GAPDH and ACTB gene transcription in 22Rv1, PM154, L-AR, LNCaP, L-rENZ and H660 cells treated with siU6atac RNA for 96 hours. Data are represented using box and whisker plots. Boxplots display values of minimum, first quartile, median, third quartile, and maximum (n=4, ordinary one-way ANOVA, 22Rv1/U6atac: *p=0.0193, 22Rv1/CoA3 spliced: *p=0.0315, LNCaP: **p=0.0017, H660: ***p=0.0003, ****p<0.0001). Each data point represents a single experiment, experiments were performed in triplicates. B) U6atac and hSCN4A, hSCN8A, p120 spliced and unspliced expression as x-fold of siScrambled (siScr.) normalized to the mean of GAPDH and ACTB gene transcription in C4-2 cells transfected with the hSCN4A, hSCN8a and p129 minigene reporter plasmids and treated with siU6atac RNA for 96 hours (hSCN4A n=3, hSCN8A n=4, p120 n=5). Data are represented using box and whisker plots.

Boxplots display values of minimum, first quartile, median, third quartile, and maximum (two-sided unpaired t-test, ns p>0.05, ***p<0.001, ****p<0.0001). Each data point represents a single experiment, experiments were performed in triplicates. C) Normalized luminescence values of minor spliceosome luc-reporter plasmids in LNCaP (siU6atac n=5, Anisomycin n=8) and PM154 (siU6atac n=5, Anisomycin n=4) cells treated with siU6atac (96 h) or Anisomycin (1 ug/ml for 4 h) (mean±SEM, ordinary one-way Anova; ns p>0.05, ***p<0.001, ****p<0.0001). Each data point represents a single experiment, experiments were performed in triplicates. D) U6atac snRNA expression as x-fold of siScrambled (siScr.) normalized to the mean of GAPDH and ACTB gene transcription in LNCaP, C4-2, 22Rv1 and PM154 cells treated with siU6atac RNA for 48 and 96 hours (n=5) Data are represented using box and whisker plots. Boxplots display values of minimum, first quartile, median, third quartile, and maximum (one-way Anova; ****p<0.0001). Experiments were performed in triplicates.

FIG. 11 shows that siU6atac triggers intron retention and AS in PCa. A-D) Overlap (left) and total number (right) of (A) upregulated protein coding genes, (B) downregulated protein coding genes, (C) upregulated MIGs, and (D) downregulated MIGs in the LNCaP, C4-2, and 22RV1 cell lines and PM154 organoid, respectively.

FIG. 12 shows that siU6atac decreases U6atac and spliced CoA3 transcript expression. U6atac snRNA, CoA3 spliced and unspliced expression as x-fold of siScrambled (siScr.) normalized to the mean of GAPDH and ACTB gene transcription in LNCaP, C4-2, 22Rv1 cells and PM154 organoids treated with siU6atac RNA for 96 hours (n=3) Data are represented using box and whisker plots. Boxplots display values of minimum, first quartile, median, third quartile, and maximum (ordinary one-way Anova; 6Uatac: *p=0.0294, ***p=0.004 (22Rv1), 0.0002 (LNCaP, ****p<0.0001; CoA3 spliced: ***p=0.0025 (PM154), 0.0035 (22Rv1), ***p=0.0003) FIG. 13 shows that siU6atac impacts protein expression in PCa. A) BRCA1, Brd9, EED, AR, NKX3.1, Rb1, AURKA, BRAF, CDN2A, CDN1A and JNK1/2 Immunoblot of LNCaP, C4-2, 22Rv1 cells and PM154 organoids treated for 96 h with siU6atac or siScrambled (scr.) B) Immunoblot quantifications. Values were normalized to GAPDH (ordinary one-way Anova, C4-2: *p=0.0485 (Rb1), 0.0196 (CDN1A); 22Rv1: *p=0.0230).

FIG. 14 shows scRNAseq in PM154 organoids. A) UMAP representation of PM154 cell line showing the clusters at the optimal resolution (0.2). B) Histogram of PM154 cell line showing the contribution of each sample in each cluster. C) UMAP representation of LNCaP cell line showing the cell cycle phase for each cell. D) Histogram of LNCaP cell line showing the percentage of cell cycle phase in each cluster. E) UMAP representation of PM154 cell line showing the AR score calculated on siScrambled and siU6atac samples. Violin representations of PM154 cell line showing the AR score calculated for each cell cycle phase. The pvalue was calculated using a Wilcoxon test. F) UMAP representation of PM154 cell line showing the EMT score calculated on siScrambled and siU6atac samples. Violin representations are showing the EMT score calculated for each cell cycle phase. The pvalue was calculated using a Wilcoxon test. G) AR score of LNCaP and PM154 cell lines calculated on siScrambled and siU6atac samples using bulk RNAseq.

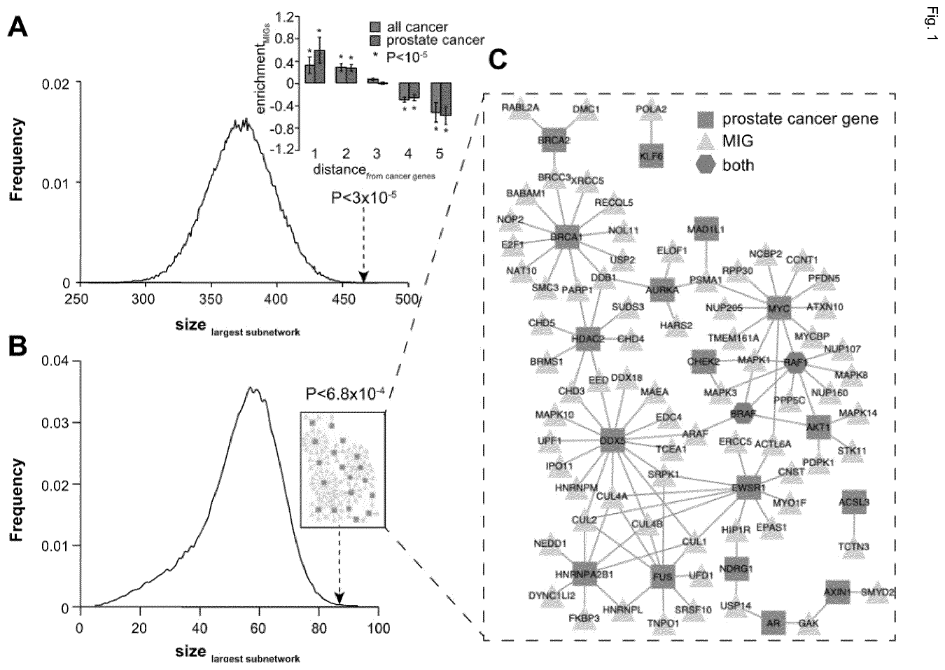

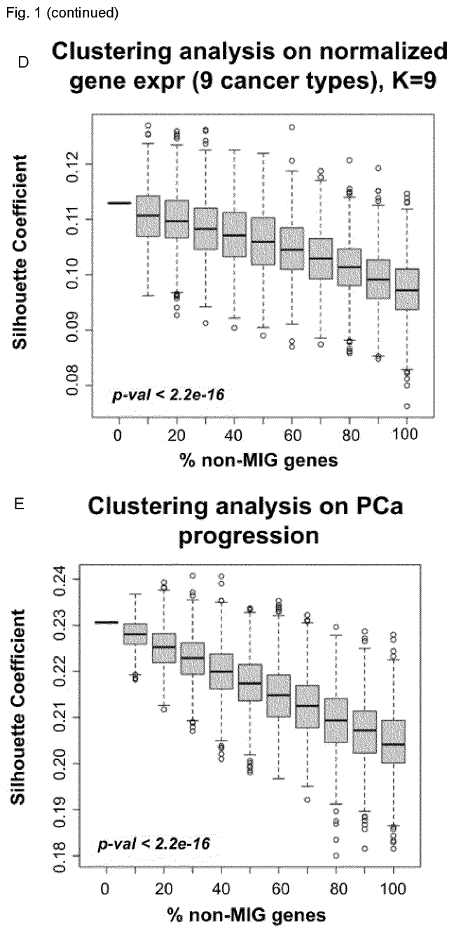

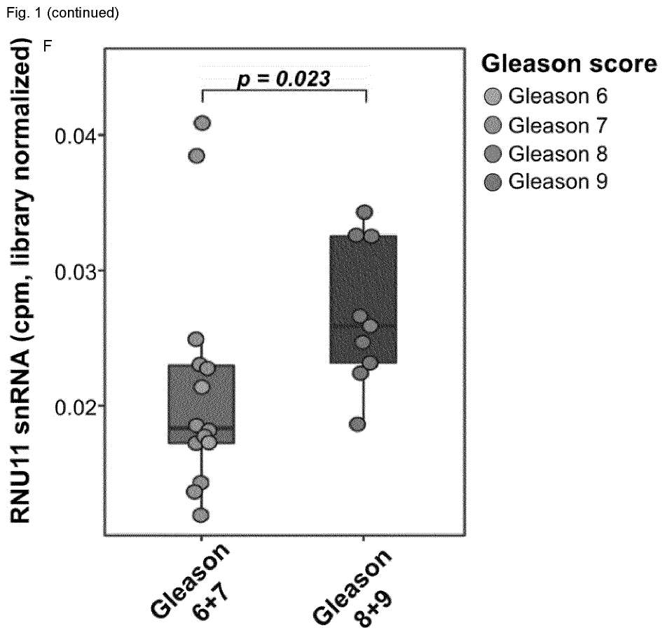

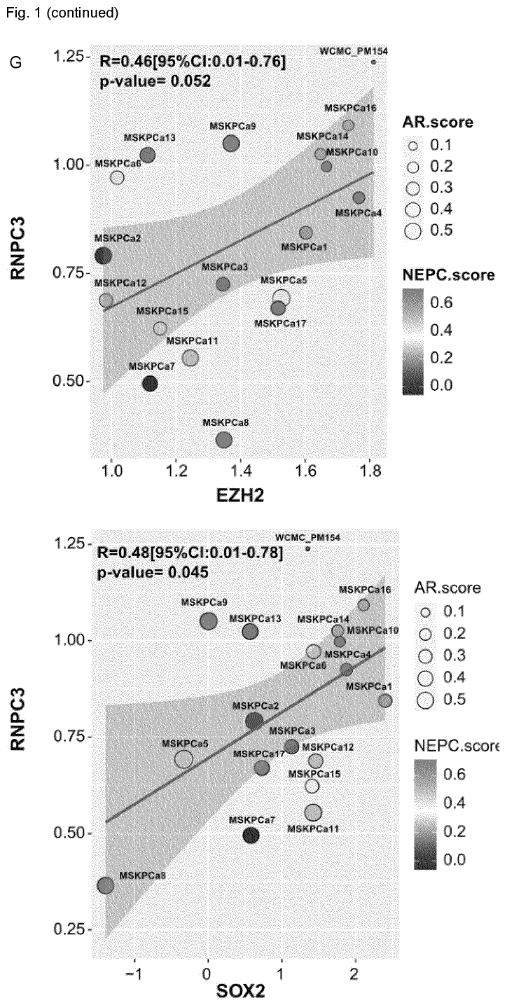

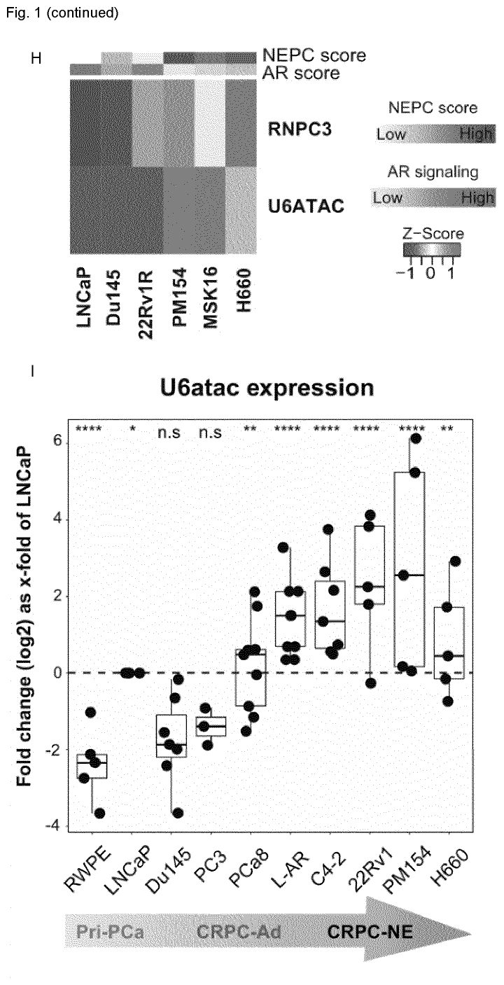

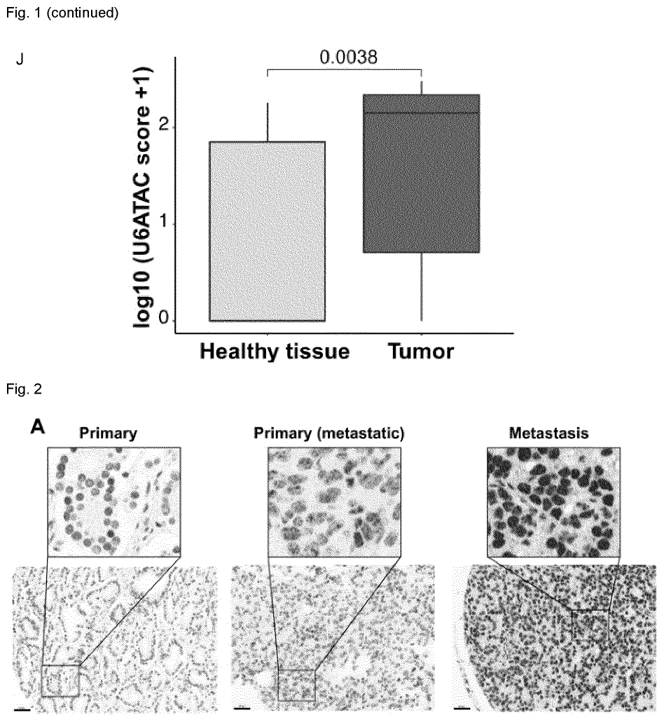

FIG. 15 shows scRNAseq analysis of MIG expression in LNCaP cells. Violin plots showing the expression Levels of selected MIGs, which represent critical PCa nodes (FIG. 1B, C) in LNCaP cells.

FIG. 16 shows scRNAseq analysis of MIG expression in PM54 organoids. Violin plots showing the expression Levels, which represent critical PCa nodes (FIG. 1B, C) in PM154 cells.

FIG. 17 shows that siU6atac and siRNPC3 impact PCa cell growth. A) U6atac snRNA and RNPC3 expression as x-fold of siScrambled (siScr.) normalized to the mean of GAPDH and ACTB gene transcription in LNCaP, L-AR, C4-2, 22Rv1, PM154, HS27 and MS2514 cells and organoids treated with siU6atac (n=5) and siRNPC3 (n=3) RNA for 96 hours. Data are represented using box and whisker plots. Boxplots display values of minimum, first quartile, median, third quartile, and maximum (two-way Anova; LNCaP: **p=0.0076, ****p<0.00′1; L-AR: ***p=0.0006, ****p<0.0001; C4-2: ns p>0.05, ****p<0.0001; 22Rv1: **p=0.0048, ****p<0.0001; PM154: **p=0.0024, ****p<0.0001; HS27: *p=0.0190, ***p=0.0005; Ms2514: **p=0.0025, ***p=0.0007). B) Growth curves of LNCaP, C4-2, L-AR, 22Rv1 cells and PM154 organoids treated for 0, 48, 72 and 96 h with siU6atac. Data is normalized to the siScrambled control and represents pooled results from 8 (siU6atac) and 6 (siRNPC3) biologically independent experiments (mean values+/−SEM, ordinary two-way Anova; ns p>0.05, **p<0.01, ****p<0.001).

FIG. 18 shows that MiS inhibition blocks viability and growth of cancer cells. A) U6atac snRNA expression as x-fold of siScrambled (siScr.) normalized to the mean of GAPDH and ACTB gene transcription in LNCaP (n=4) and L-AR (n=8) cells treated with siU6atac RNA for 96 hours. Data are represented using box and whisker plots. Boxplots display values of minimum, first quartile, median, third quartile, and maximum (ordinary one-way Anova, ****p<0.0001). B) Growth curves of LNCaP and L-AR cells (n=3, mean+/−SEM, ordinary two-way Anova; ****p<0.00001).C) U6atac and U12 snRNA and RNPC3 and PDCD7 expression as x-fold of siScrambled (siScr.) normalized to the mean of GAPDH and ACTB gene transcription in C4-2, DLD1, HEK 293, K-562, LN18, MDA-MB-231 and UMOC-9 cells treated with siU6atac, siPDCD7, siRNPC3 and siU12 RNA for 96 hours (n=3). Data are represented using box and whisker plots. Boxplots display values of minimum, first quartile, median, third quartile, and maximum (unpaired two-tailed t-test; p-values indicated in figure). D) Cell viability assay measured using CellTiter-GLO in in C4-2, DLD1, HEK 293, K-562, LN18 and MDA-MB-231 treated cells treated with siPDCD7, siRNPC3 and siU12 RNA (48, 72, 96 and 120 h). Data is normalized to the siScrambled (siScr.) control and represents pooled results from 4 biologically independent experiments (mean values+/−SEM, two-way Anova; p-values indicated in figure) E) U6atac snRNA, CoA3 spliced and unspliced expression after siU6atac (96 h) treatment and EZH2 expression after siEZH2 (96 h) treatment as x-fold of siScrambled (siScr.) normalized to the mean of GAPDH and ACTB gene transcription in LNCaP (n=4), C4-2 (n=4) and L-rENZ (n=6) cells. Data are represented using box and whisker plots. Boxplots display values of minimum, first quartile, median, third quartile, and maximum (unpaired two-tailed t-test; p-values indicated in figure). E) U6atac snRNA, CoA3 spliced and unspliced expression as x-fold of siScrambled (siScr.) normalized to the mean of GAPDH and ACTB gene transcription in MSK10 (n=3), MSK14 (n=3), MSK16 (n=5), MSK8 (n=3) and PM154 (n=4) patient derived PCa organoids treated with siU6atac RNA for 96 hours. Data are represented using box and whisker plots. Boxplots display values of minimum, first quartile, median, third quartile, and maximum (unpaired two-tailed t-test; p-values indicated in figure).

FIG. 19 shows that MiS inhibition has stronger impact on cancer than on normal cells. Brightfield microcopy of C4-2, HS27, MS2514 and MS2513 cells 96 hours after siU6atac and siRNPC3 treatment. Cells treated with Scrambled siRNA are shown as control. Scale bars (red): 100 μm.

FIG. 20 shows that minor splicing correlates with PCa progression. CoA3 spliced as x-fold of unspliced expression normalized to the mean of GAPDH and ACTB gene transcription in RWPE (n=6), LNCaP (n=5), Du145 (n=6), PC3 (n=3), PCc8(n=4), L-AR (n=3), C4-2 (n=8), 22Rv1 (n=5), PM154 (n=5), MSK16 (n=1) and H660 (n=4) cells and organoids. Each data point represents a single experiment, experiments were performed in triplicates

FIG. 21 shows MIG expression in LNCaP, C4-2, 22Rv1 and PM154 cells. heatmap showing log 10 transformed TPM values. Color denotes percentile (10th-90th) with blue low expression and red high expression. UpSig lists show genes significantly more highly expressed in one cell line when compared to all other cell lines via ANOVA (P<0.05) with post-hoc Tukey's test (P<0.05) using BH P-value adjustment.

FIG. 22 shows that p38MAPK regulates MiS in PCa-NE. A) Normalized luminescence values of minor/major spliceosome luc-reporter plasmids in LNCaP n=8/3, C4-2 n=6/4, Pm154 n=5/3), mean±SEM, two-way Anova; ns p>0.05, *p<0.05, ****p<0.0001). Each data point represents a single experiment; experiments were performed in triplicates. B and C) Normalized luminescence values of minor/major spliceosome luc-reporter plasmids in LNCaP C4-2 22Rv1 and Pm154 cells (n=3) treated with siScrambled or siMAPK13/14 for 96 h, mean±SEM, one-way Anova; ns p>0.05, ****p<0.0001). Each data point represents a single experiment; experiments were performed in triplicates. Lower panel represents the KD control: MAPK13/14 snRNA expression as x-fold of siScrambled normalized to the mean of GAPDH and ACTB gene transcription. Data are represented using box and whisker plots. Boxplots display values of minimum, first quartile, median, third quartile, and maximum (one-way Anova, ns; not significant, p>0.05, *p<0.05, **p<0.01, ***p<0.001). Experiments were performed in triplicates. D) schematic overview of U6atc turnover regulation. AR blocks U6atac turnover in CRPC which leads to increase MiS activity while p38 does so in NEPC.

FIG. 23 shows Relative performance of MIGs and non-MIGs with respect to clustering gene expression data from. A) nine (Biliary-, Breast-, Colorectal-, Lung-, Ovary-, Pancreas-, Stomach-, Thymus-, and Prostate-Adenocarcinoma) and B) 23 distinct cancer types from the PCAWG consortium. Each box corresponds to a 1000-length simulation in which non-MIGs are randomly sampled from among all non-MIGs in the genome. The plots exhibits a positive relationship between the relative abundance of MIGs and the quality of clustering, as quantified using the Silhouette coefficient. The P-value is based on a two-sided t-test in which each sample value is taken to be the difference between the Silhouette coefficient of one of the 1000 100% non-MIG samples and the corresponding Silhouette coefficient when only MIGs are used for clustering (i.e., the Silhouette coefficient corresponding to 0% non-MIGs).

FIG. 24 shows U11, U12, U4atac and U6atac expression as x-fold of the respective LNCaP and PC3 expression values normalized to the mean of GAPDH and ACTB gene transcription in L-AR (n=5), L-rENZ (n=4) and PC3Pro4 cells (n=5). Data are represented using box and whisker plots. Boxplots display values of minimum, first quartile, median, third quartile, and maximum (ordinary one-way Anova, ns p>0.05, *p<0.05). Each data point represents a single experiment, experiments were performed in triplicates.

FIG. 25 shows MiS influences REST splicing: A) canonical REST and REST4 expression as x-fold of LNCaP cells normalized to the mean of GAPDH and ACTB gene transcription in LNCaP, C4-2, 22Rv1 and PM154 cells (n=4, one-way Anova, ns p>0.05, *p<0.05, **p<0.01). Each data point represents a single experiment; experiments were performed in triplicates. B) Scatterplots showing the relationship between the expression levels of REST/REST4 genes versus the AR/NEPC scores (n=249, Pearson correlation test). Boxplots showing the expression levels of REST/REST4 genes of the samples AR versus NEPC positive (score >=0.4, Wilcoxon test). C) canonical REST expression after siU6atac treatment as x-fold of siScrambled ctr cells normalized to the mean of GAPDH and ACTB gene transcription in LNCaP (n=4), C4-2 (n=5), 22Rv (n=6) and PM154 (n=3) cells (one-way Anova, ns p>0.05, *p<0.05, ***p<0.001). Each data point represents a single experiment; experiments were performed in triplicates. D) bulk RNAseq TPM values of REST4 and REST? isoforms of LNCaP, C4-2, 22Rv1 and PM154 cells treated for 48 h with siU6atac and siScrambled. Red font indicates cases with a fold change threshold >1.5 E) REST4 expression after siU6atac (48-72 h) treatment as x-fold of siScrambled ctr cells normalized to the mean of GAPDH and ACTB gene transcription in LNCaP, C4-2, 22Rv and PM154 cells (n=5, one-way Anova, ns p >0.05, *p<0.05). Each data point represents a single experiment; experiments were performed in triplicates. F) product of a qRT-PCR with primers binding to exon 3 (fw) and exon 4 (rv) of REST loaded on a 3% agarose gel. Experiment performed in LNCaP, C4-2, 22Rv1 and PM154 cells treated with siScrambled or siU6atac (48 h). canonical REST size expected at 150 bp and REST4 sample size expected at 200 bp.

FIG. 26 shows AR and minor splicing. A) U6atac expression as x-fold of LNCaP cells normalized to the mean of GAPDH and ACTB gene transcription in LNCaP and L-rENZ cells and in L-AR cells that were treated with siSCrambled, siAR (n=2), c/s media and/orDHT (n=4) (unpaired t-test, *p<0.05). Each data point represents a single experiment; experiments were performed in triplicates. B) Immunoblot showing expression levels of PARP (MIG control, n=5), EZH2 (n=8), Rb1 (n=3) and AR (n=8) in LNCaP and C4-2 cell lines treated for 96 hours with siScrambled or siU6atac RNA. GAPDH was used as loading control (multiple unpaired t-test, ****p<0.001).

FIG. 27 shows A) confluence (y-axis) and B) viability (y-axis) of various cell lines treated with siU6atac/siScrambled for 120 h (n=3). Cells are ranked according to their doubling time (DT). Normal cells are depicted in green. C) U6atac expression as x-fold of siScrambled after siU6atac treatment of all cells used for that experiment. Each data point represents a single experiment; experiments were performed in triplicates. Data are represented using box and whisker plots. Boxplots display values of minimum, first quartile, median, third quartile, and maximum.

FIG. 28 shows A) Growth curves of mCherry positive HS27 cells and GFP positive C4-2 cells grown in a co-culture which was treated with siScrambled or siU6atac. Data represented as x-fold of siScrambled. Data represents pooled results from biologically independent experiments (n=3, mean+/−SEM, ordinary two-way Anova; ns p>0.05, *p<0.05) B) U6atac expression as x-fold of siScrambled after siU6atac treatment of all cells used for that experiment. Each data point represents a single experiment; experiments were performed in triplicates. Data are represented using box and whisker plots. Boxplots display values of minimum, first quartile, median, third quartile, and maximum (n=3, mean+/−SEM, ordinary one-way Anova; ****p<0.0001)

FIG. 29 shows rescue experiments with a hairpin against RNU6atac that proved dependence of critical MIG expression on U6atac.

FIG. 30 shows A) Brightfield and Fluorescent microscopy of C42′U6atac cells (Scale=50 uM) B) U6atac expression of C4-2 EV and C42′U6atac cells (n=3). Each data point represents a single experiment; experiments were performed in triplicates. Data are represented using box and whisker plots. Boxplots display values of minimum, first quartile, median, third quartile, and maximum (n=3, mean+/−SEM, ordinary one-way Anova; *p<0.05) C) Growth curves of C4-2′U6atac cells (blue) and C4-2 cells treated with siU6atac (pink) r. Data represented as x-fold of C4-2′EV or siScrambled respectively. Data represents pooled results from biologically independent experiments (n=3, mean+/−SEM, ordinary two-way Anova; ****p<0.0001) D) experimental set-up of rescue experiment E) 1% Agarosegel showing amplified endogenous U6atac (1427 bp) and CRISPERed U6atac (707 bp) in C4-2 and C4-2′U6atac cells which were untreated (NTC), transfected with empty backbone vector Px458 or with gRNAs targeting U6atac (CRISPR) F) U6atac expression of C4-2 and C42′U6atac cells (n=3) which were treated with gRNAs against U6atac. Data represented as x-fold of cells treated with empty vector Px458 Each data point represents a single experiment; experiments were performed in triplicates Data are represented using box and whisker plots. Boxplots display values of minimum, first quartile, median, third quartile, and maximum. (n=3, mean+/−SEM, ordinary one-way Anova; ***p<0.001)G) Brightfield microscopy of C4-2 and C4-2′U6atac cells treated with either empty vector Px458 or gRNAs against U6atac (Scale=50 uM) H) Viability of C4-2 and C4-2′U6atac cells treated with gRNAs against U6atac. Data represented as x-fold of cells treated with empty vector Px458 (n=2, experiments performed in triplicates).

FIG. 31 shows Pearson correlation analysis between SNRNP48, SNRNP25, SNRNP35, UBE2K, ZMAT5, PDCD7 (MiS proteins), ZRSR2, CENPA (MiS and major spliceosome proteins) and EZH2. Organoids are color-coded according to their transcriptomic NEPC score, whereby a score >0.4 indicates a CRPC-NE phenotype while a score<0.4 indicates a CRPC-Adeno phenotype (Beltran et al., PMID 26855148). Organoids are size-coded according to their AR-score.

### EXAMPLES

### Example 1: MIGs Play a Crucial Role in Cancer Onset and Progression

Since MIGs are highly enriched in cell cycle regulation and survival, the inventors explored whether MIGs are enriched in biological pathways exploited by cancer-causing genes. For this, the inventors determined whether MIG-encoded proteins were enriched amongst proteins that interact with proteins encoded by cancer-causing genes in a network of 160,881 protein-protein interactions (PPI) between 15,366 human proteins as of the HINT database (Das, J et al., BMC Syst Biol 2012, 6, 92). The inventors hypothesized that MIGs are prime recipients of molecular information that cascade from cancer-causing genes through their protein-protein interactions. Considering 403 cancer-causing genes in the set of 15,366 interacting proteins as of the Cancer Genome Interpreter database (Bigas, N et al., Genome Medicine 2018, 10 (1), 25), the inventors computed the shortest distance d of all proteins encoded by cancer-causing genes, where d=1 indicates a direct interaction. In each distance bin, the inventors determined the enrichment of 542 MIG-encoded proteins (inset, FIG. 1A) to find significant enrichment of MIGs in the immediate network neighbourhood of cancer-causing genes (permutation test: d=1,2; p<10E-5). However, MIGs were significantly diluted at higher distances (d >3, p<10E-5) (inset, FIG. 1A). Next the inventors focused on immediate interactors of the proteins encoded by 26 PCa-causing genes (inset FIG. 1A) (Bigas, N et al. ibid). The inventors found a significant enrichment of MIG-encoded proteins as immediate interactors of these 26 PCa proteins. Since MIGs were significantly enriched at d=1 from cancer-causing genes, the inventors next identified 3 PPI subnetworks of interactions between MIG encoded proteins and the 26 PCa proteins. In fact, the inventors found that MIG-encoded proteins formed a significantly large (permutation test, p<6.8×10E-4) tight-knit web of interactions between 17 PCa proteins and 72 MIG-encoded proteins, capturing more than 95% of proteins (FIG. 1B, C). Similarly, significant (permutation test, p<3×10E-5) large tight-knit subnetworks were obtained for MIG-encoded proteins and proteins encoded by all 403 cancer-causing genes (FIG. 1A). Such findings suggest that proteins encoded by cancer-causing genes in general and PCa causing-genes in particular tend to significantly interact with MIG-encoded proteins in the underlying human interaction network.

That MIGs interact directly with cancer-causing proteins led the inventors to explore the extent to which MIGs exhibit a greater degree of differential expression between distinct cancer types (relative to non-MIGs). To address this question, the inventors first used normalized MIG expression data to perform hierarchical sample clustering (HSC) of 9 different solid cancer types, and then evaluated the extent to which this MIG expression successfully clusters samples by cancer type. The inventors first evaluated the quality of the resultant clustering by visualizing the clustered data with a dendrogram and its associated heatmap, wherein structure can readily be seen in the case for which only MIGs were used in clustering the samples (FIG. 1D). To evaluate whether MIGs more successfully cluster the data relative to non-MIGs, the inventors performed the same type of visual analysis for datasets comprising different relative abundances of MIGs and non-MIGs. Specifically, this controlled approach entailed progressively polluting the pool of MIGs with increasing samples of non-MIGs, such that 0%, 10%, 20%, 30%, . . . 100% of all genes in a given gene set were non-MIGs. In adopting this approach, the inventors found that these progressive increases in the relative abundances of non-MIGs (within this set of genes used for clustering) gave rise to progressively deteriorating quality in data clustering. This can be seen visually by observing the deteriorating structure in the heatmaps associated with these various relative abundances of non-MIG.

The inventors then took a more objective, quantitative approach to measure this deterioration in clustering (i.e., visual structure within these heatmaps). To quantitatively evaluate the extent to which a given expression dataset may be clustered, the inventors used the Silhouette coefficient, wherein higher coefficient values designate more clearly-defined and correct clustering. The inventors employed a simulation (data re-sampling-based scheme) to generate 1000 gene sets for each fraction of non-MIG genes, and then calculated the Silhouette coefficient associated with each resampled gene set. This quantitative approach recapitulated what the inventors had observed visually in the heatmaps: the Silhouette coefficients associated with clustering expression data in gene sets with higher fractions of non-MIGs resulted in less effective in clustering for these 9 different cancer types (FIG. 1E; two-sided t-test p<2.2E-16). That MIGs enable better clustering performance than do non-MIGs suggests that MIG expression is more informative for defining differences between these 9 cancer types.

The inventors next evaluated whether MIG expression may likewise exhibit greater differential gene expression across different stages of PCa progression (relative to non-MIGs). This was carried out for the transdifferentiation analysis across the distinct transcriptomes, illustrated by principal component analysis. Specifically, to obtain data representing different stages of PCa progression, the inventors used prostate samples derived from GTEx (normal tissues), TCGA (primary PCa samples), and SU2C (CRPC-adeno and CRPC-NE) datasets. As with the inventors' clustering analysis on 9 distinct cancer types detailed above, the inventors observed that MIG expression resulted in better clustering performance on these distinct phases of prostate cancer progression, although the visual disparities between the heatmaps associated with 0% non-MIGs and 100% non-MIGs are not as pronounced as what the inventors had observed in the context of the inventors' pan-cancer analysis. The same simulation-based scheme in which Silhouette coefficients were measured in the inventors' pan-cancer analysis was similarly applied in the context of these broad stages of cancer progression. Again, the inventors observed that MIG expression results in greater clustering performance than non-MIG expression (FIG. 1F and G; two-sided t-test p<2.2E-16). Together these findings show that transcription of MIGs is closely associated to cancer onset and progression.

### Example 2: MiS Component Expression Correlates with PCa Progression

The upregulation of MIGs with PCa progression and transdifferentiation would necessitate increased MiS activity, which in turn is regulated by the levels of MiS components. Therefore, the inventors queried the expression kinetics of U11 snRNA (RNU11), a crucial MiS component in PCa (PRAD) TCGA data. The inventors observed a significant (Wilcoxon Test, p=0.023) association between high-grade (Gleason score 8 and 9) PCa patients and high RNU11 expression (FIG. 1). Another unique MiS component is the protein RNPC3, which has been linked to cancer-associated scleroderma. Therefore, the inventors queried RNA-seq data from 18 PCa organoids (CRPC-adeno and CRPC-NE), which revealed that expression of RNPC3 showed a tendency towards positive correlation with the pluripotent stem cell and early differentiation markers SOX2 and epigenetic regulator EZH2, respectively (FIG. 1). The inventors observed RNPC3 expression paralleled PCa disease progression with the lowest expression in benign and hormone-sensitive PCa cells (LNCaP), intermediate expression in aggressive CRPC-adeno cells (22Rv1) and the highest expression in CRPC-NE cells (H660) and patient-derived organoid lines (WCM154 and Msk16) (FIG. 1). The same was observed for U6atac snRNA which is regulated by high turnover rate (and is therefore a crucial regulatory node of MiS activity). High RNU6atac and RNPC3 expression further correlated with high NEPC and low AR scores, which are indicators for poor PCa prognosis due to CRPC-NE lineage plasticity (FIG. 1). Moreover, low U6atac levels inherently throttle MiS activity, so its increase in expression is especially informative. Indeed, quantitative-RT-PCR data confirmed that MiS component expression correlates with PCa progression: expression of the four MiS snRNAs—U6atac, U4atac, U11 and U12—was higher in CRPC-adeno and CRPC-NE cell lines relative to hormone-responsive LNCaP and RWPE cells or to AR-low Du145 and PC3 cells (FIG. 1).

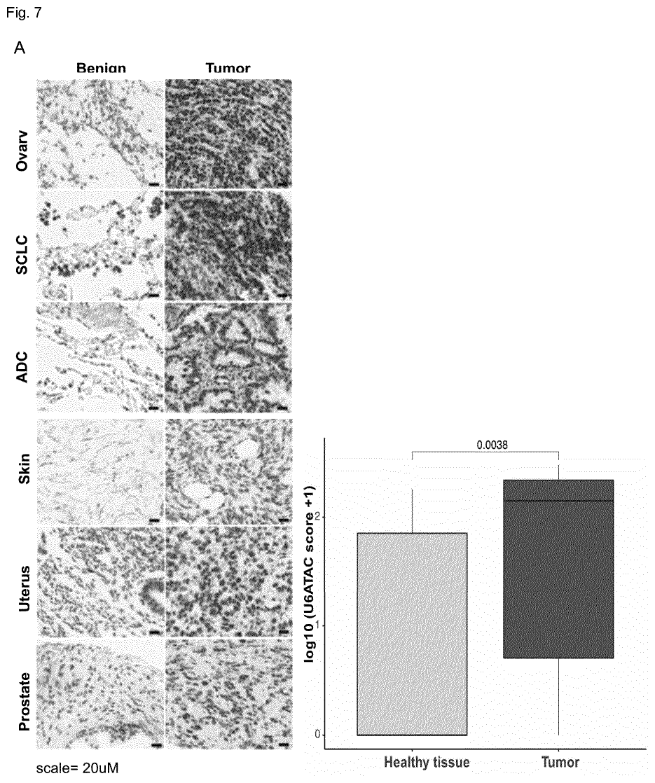

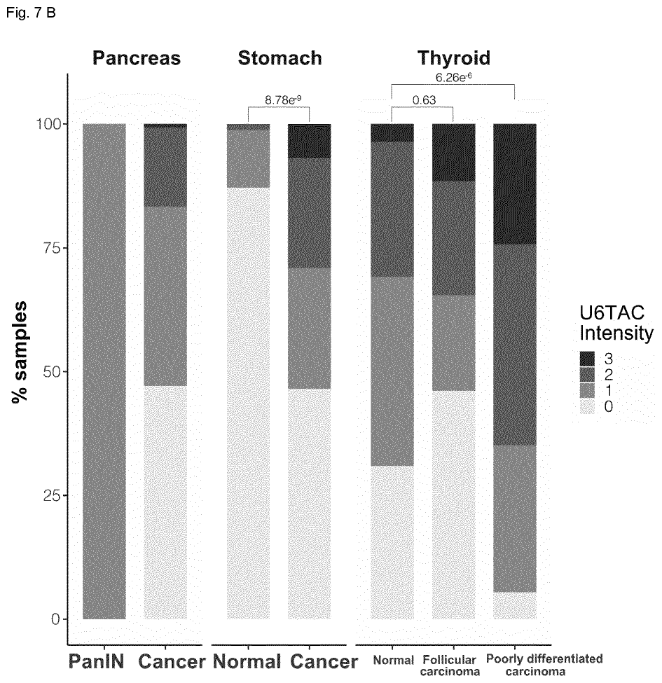

To test the prediction that U6atac expression is elevated with cancer onset and progression, the inventors developed an in-situ probe specifically against U6atac. The inventors used this probe to survey different cancer types, and found significantly higher expression of U6atac in cancer tissue compared to matching benign samples (Wilcox test, p=0.0038). This finding demonstrates a strong specificity of U6atac expression for highly proliferative tissues (pan-cancer Tissue Microarray (TMA)) (FIG. 1 and FIG. 7). Consistently, thyroid and colon cancer showed a higher U6atac intensity score in cancer compared to the corresponding benign tissues (FIG. 7). Together the inventors' data underscore that expression of both MiS components and MIGs show a reliable association with cancer onset and progression.

### Example 3: MiS Activity Correlates with PCa Progression

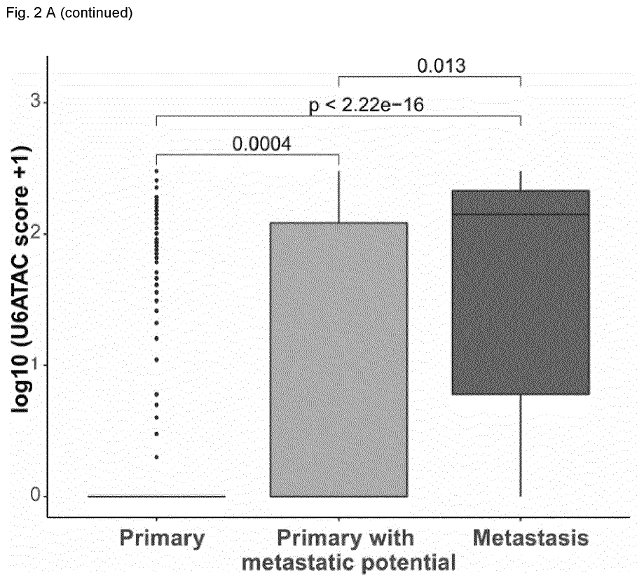

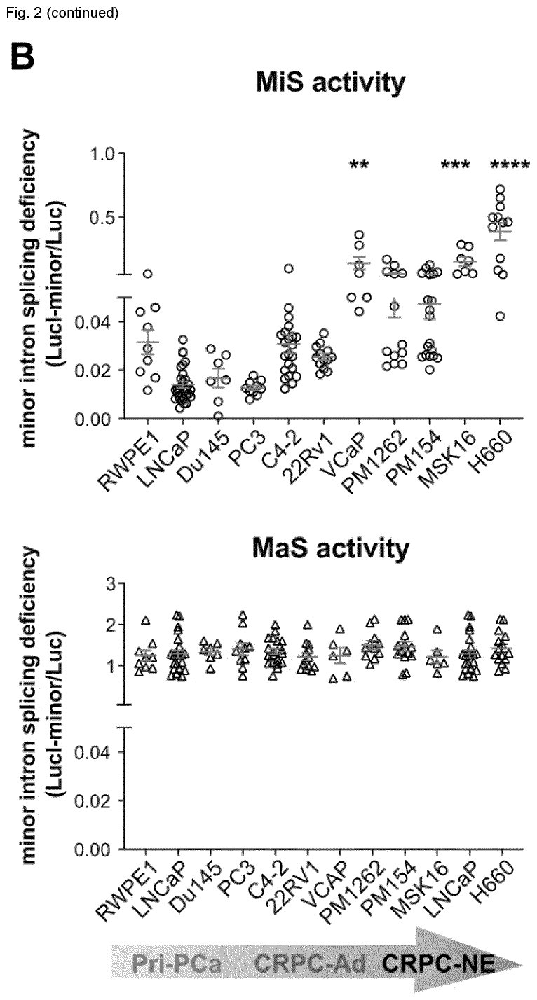

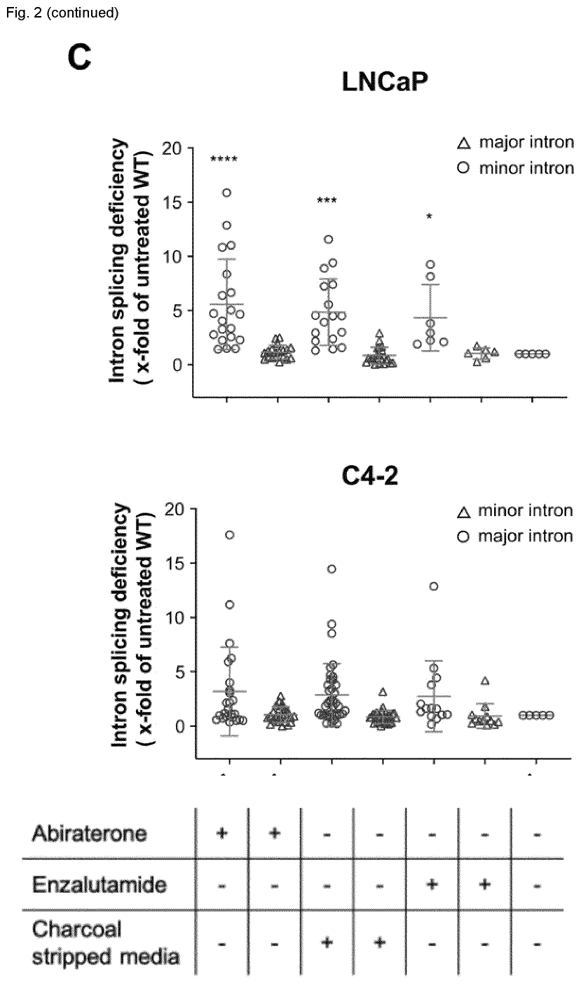

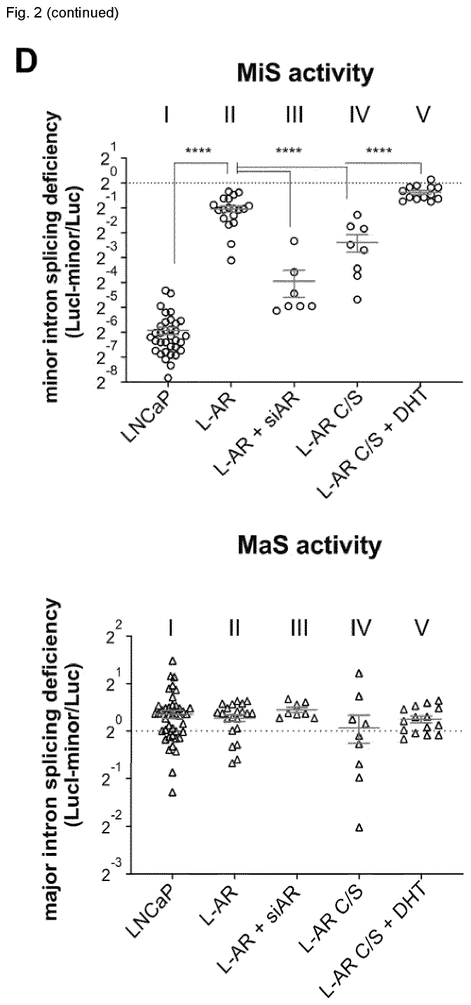

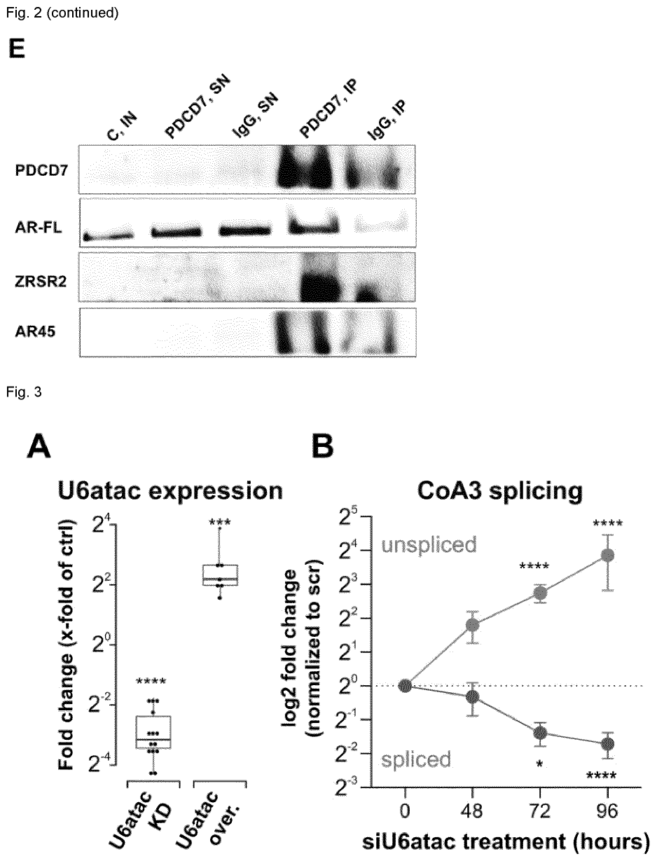

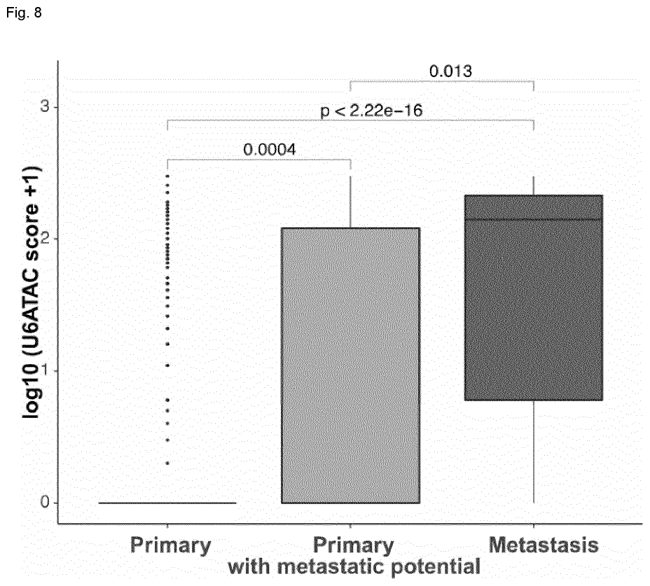

Consistent with the increased expression of U6atac in more aggressive PCa cell lines (FIG. 1), the inventors also observed significantly increased U6atac expression in primary and metastatic PCa patient samples compared to benign prostate (Wilcox test, p=0.0004). More importantly, the inventors discovered that metastatic PCa had significantly elevated U6atac expression compared to primary PCa (Wilcox test, p=0.013) (FIG. 2A and FIG. 8). Thus, the inventors posited that MiS activity should increase with PCa progression. To test this, the inventors used minor and major splicing reporter plasmids (kindly provided by the Dreyfuss Lab) in different PCa cell lines reflecting the range of PCa progression and transdifferentiation. The inventors found a trend towards more efficient minor intron splicing in therapy-resistant C4-2 and 22Rv1 cells compared to the hormone-sensitive LNCaP cells (FIG. 2B) or AR-low DU145 and PC3 cells. The inventors also observed a six-fold increase in minor intron splicing in the CRPC-NE cell and patient-derived organoid lines (PM-154, PM1262, MSK16 and NCI-H660) (FIG. 2B). In contrast, major intron splicing was unaffected in all cell and organoid lines tested (FIG. 2B) indicating that, unlike the major spliceosome, minor spliceosome activity dynamically increases with PCa progression. This led the inventors to explore the molecular mechanisms underlying the regulation of MiS activity in PCa.

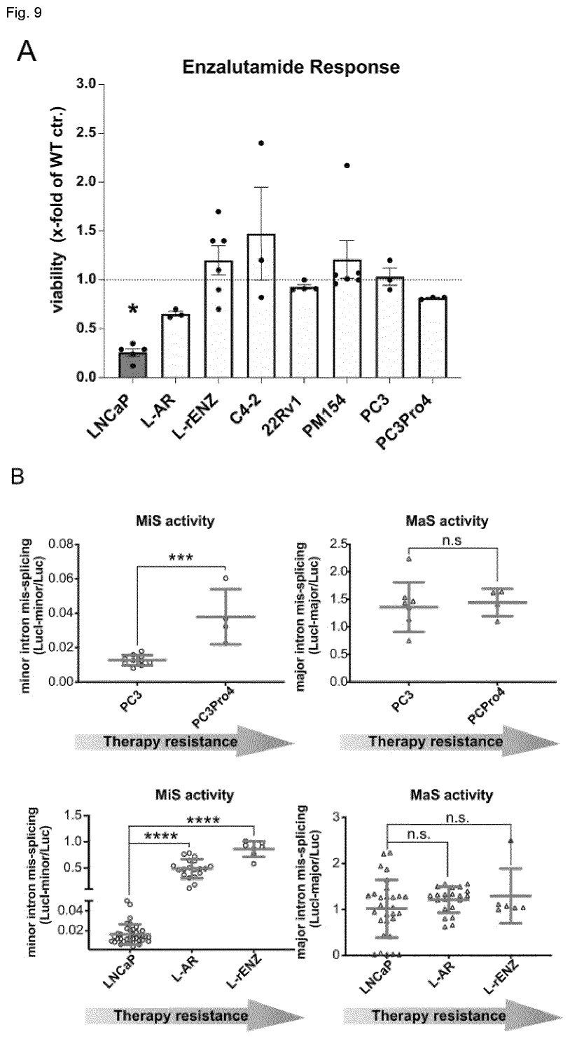

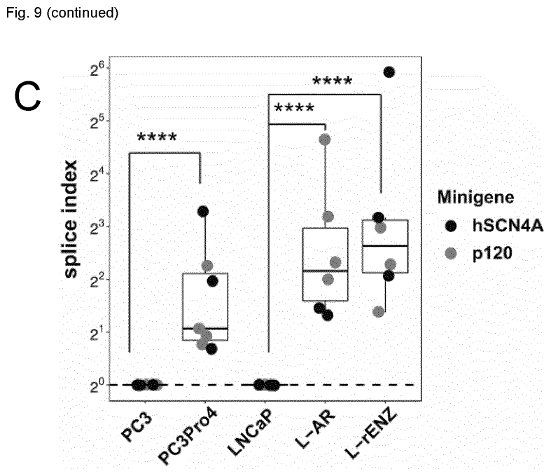

AR signaling plays a critical role in PCa progression, and is often the apex of oncogenic pathways. Therefore, the inventors hypothesized that MiS activity across PCa progression might be linked to AR signaling. To simulate stress response and re-activation of AR signalling in PCa, the inventors mimicked therapy resistance mechanisms, and subjected PCa cells to long term androgen deprivation therapy (ADT) and ARSi using charcoal-stripped (C/S) media, abiraterone and enzalutamide. The inventors observed a significant increase in MiS activity in cells exposed to ADT/ARSi, whereas the treatment had only limited effects on major splicing (one-way ANOVA; *p=0.0117, ***p and ****p<0.001) (FIG. 2C). A similar association was observed in PC3 cells and their more highly differentiated subline PC3Pro4 as well as in hormone-sensitive LNCaP cells and a subline which overexpresses AR (L-AR and/or is ENZ-resistant (L-rENZ) (FIG. 9A and B). PC3Pro4, L-AR and L-rENZ displayed significantly increased MiS activity but unchanged major spliceosome activity (FIG. 9B) (one-way ANOVA; ***p and ****p<0.001). This increase in MiS activity in those lines was further confirmed by the minor-intron containing p120 and hSCN4A minigene constructs. The inventors observed that L-AR, L-rENZ- and PC3Pro4-cells, compared to their native LNCaP and PC3 lines, displayed a higher splicing efficiency in both reporter assays (FIG. 9C). Lastly, both L-rENZ and PC3Pro4 cells expressed higher MiS snRNA levels (FIG. 24) compared to their respective WT lines.

Next, the inventors explored the relationship of AR signaling to minor intron splicing. Here, the inventors used luciferase reporter as the readout of minor intron splicing in therapy-sensitive LNCaP cells. The overexpression of AR led to a significant increase in MiS activity (FIG. 2D II) (one-way Anova; ****p<0.001), which was not observed with the major spliceosome reporter (FIG. 2D II). MiS activity decreased upon siRNA against AR (FIG. 2D III). AR KD was confirmed by qRT-PCR. To further explore the regulation of MiS activity by AR signaling, the inventors treated L-AR cells with hormone-depleted media to block AR activation, which was confirmed by a reduction in KLK3 expression. Again, the inventors found that blocking AR activation resulted in decreased MiS activity (FIG. 2D IV). Finally, addition of dihydrotestosterone (DHT) to the hormone-depleted media rescued MiS activity to levels observed in L-AR alone (FIG. 2D V). Similar findings were observed with U6atac snRNA expression and the p120 minor intron splicing minigene reporter. In contrast, major spliceosome activity was not affected by AR modulation (FIG. 2D).

The association between AR activation and MiS activity suggests the existence of an AR-MiS regulatory axis. To address this, the inventors determined whether AR interacts with MiS proteins. Co-immunoprecipitations (Co-IPs) with an antibody directed against the AR revealed an interaction with PDCD7, one of the seven proteins unique to the MiS complex, and vice versa, implying a direct regulation of the MiS by AR through PDCD7 (FIG. 2E). AR45, an AR splice variant known to heterodimerize with the full-length version, and ZRSR2, which is known to interact with the MiS were used as positive controls. Further support was obtained by proximity ligation assays demonstrating an in-situ interaction between AR and PDCD7 as well as with the AR interactor HSP90 (FIG. 2F). Taken together, this data suggest that MiS activity is regulated by AR signalling, it can be stimulated by ADT, and that it increases with PCa disease progression to CRPC-adeno and CRPC-NE.

### Example 4: MiS Inhibition in Prostate Cancer Results in Aberrant Minor Intron Splicing

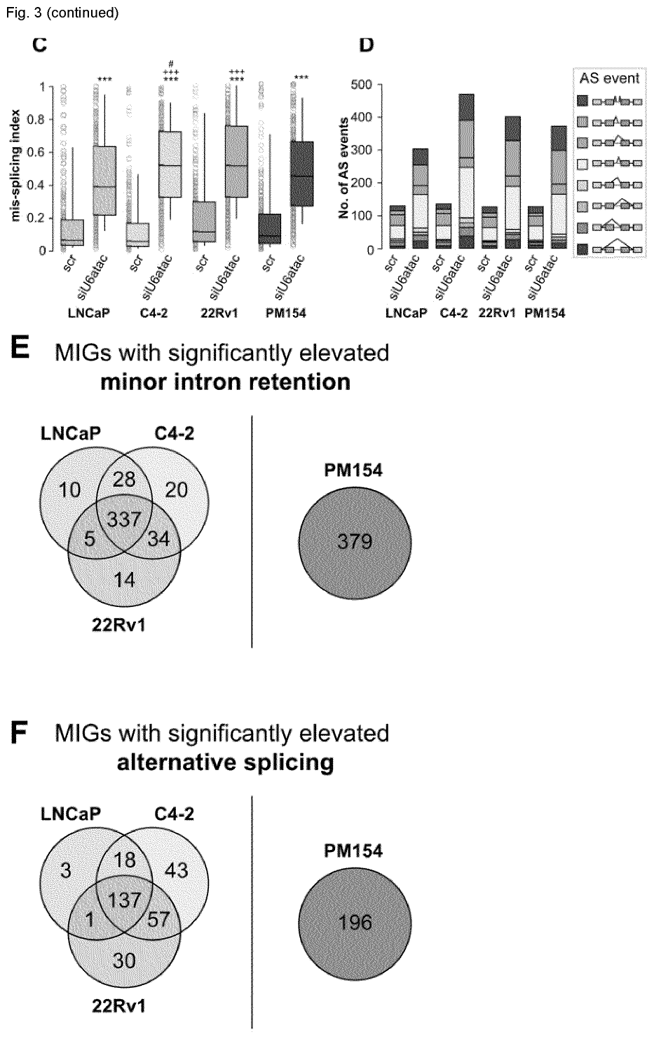

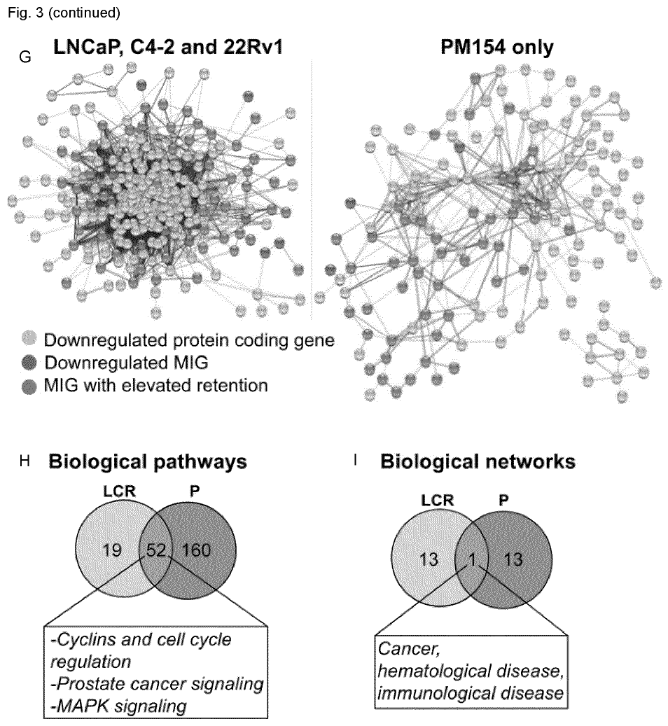

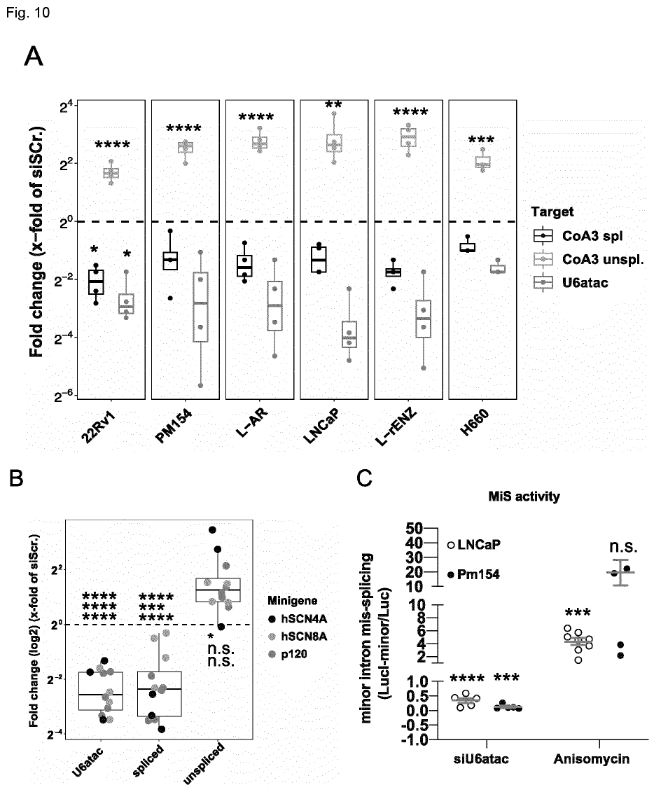

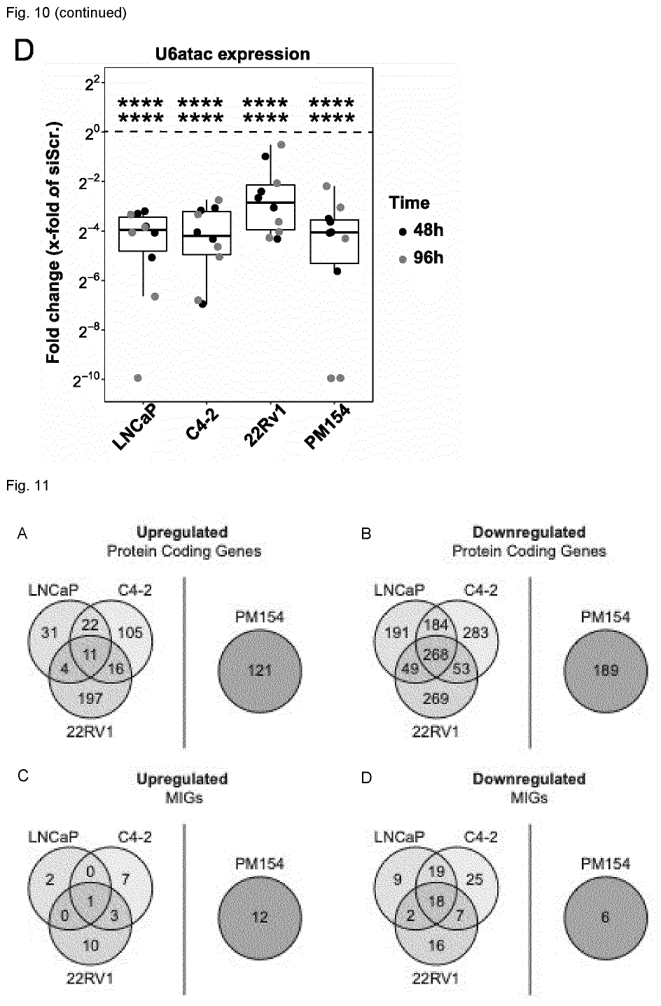

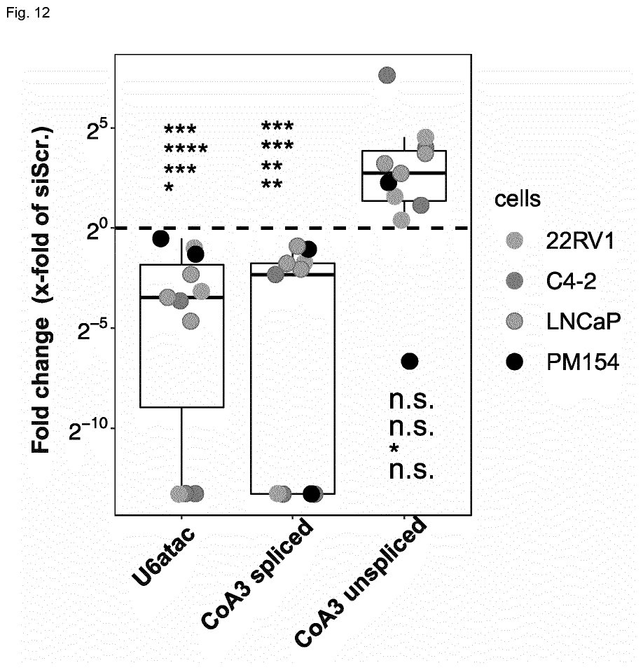

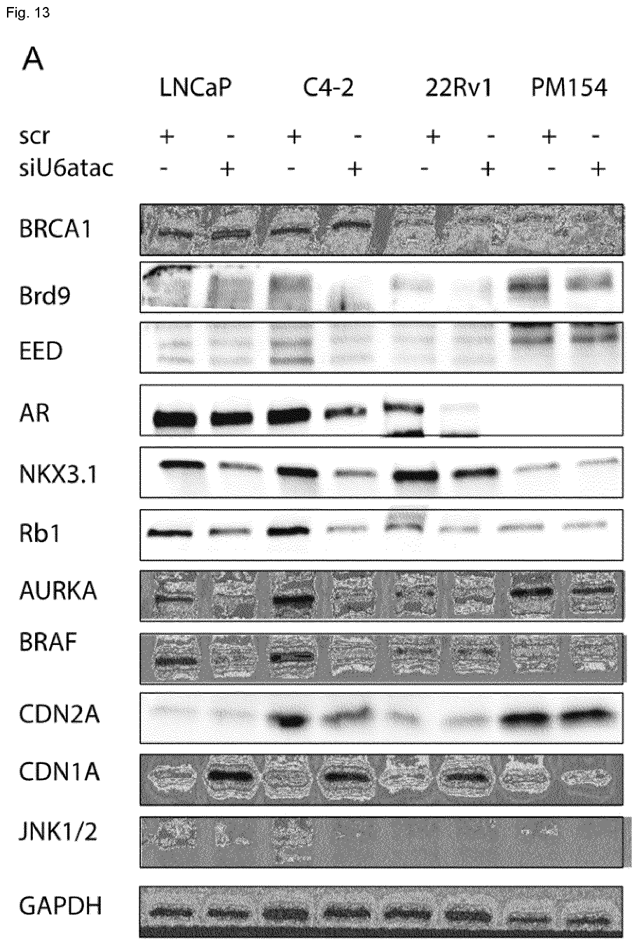

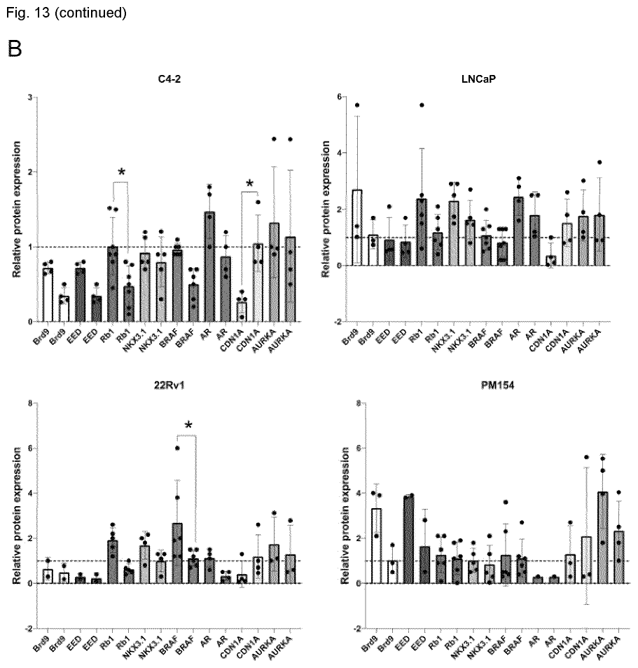

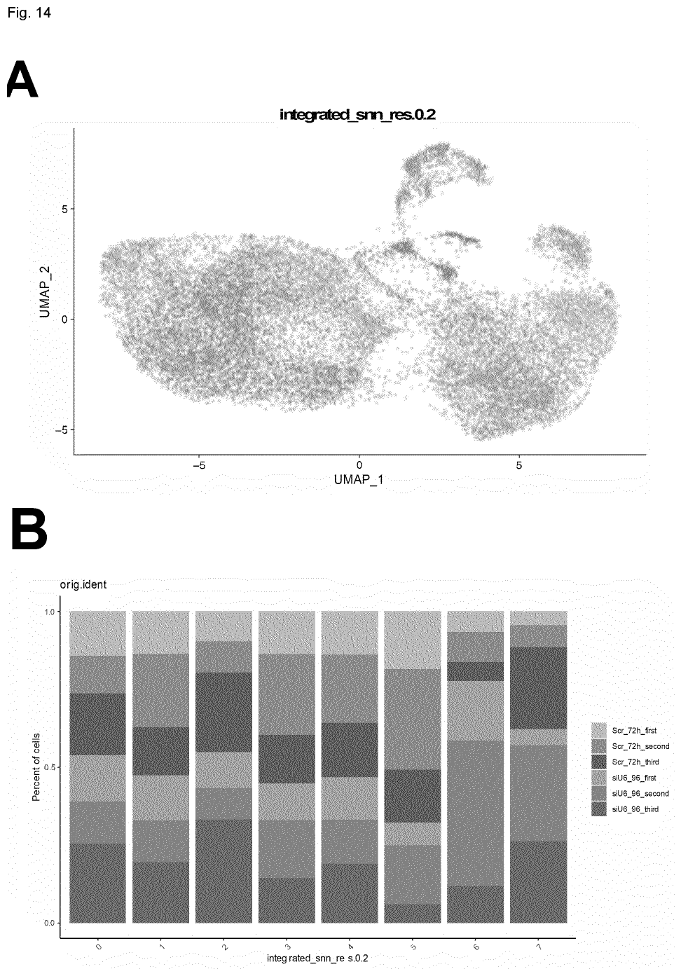

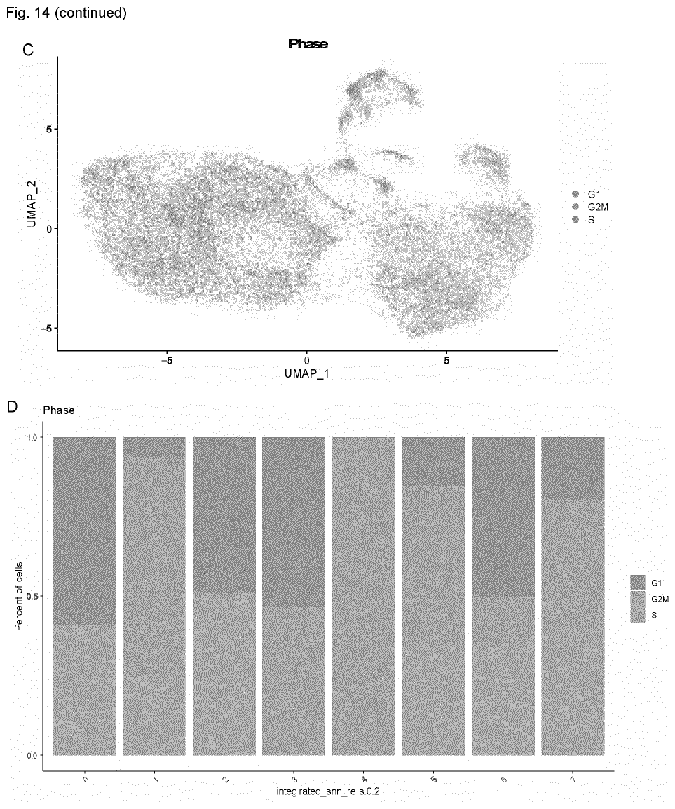

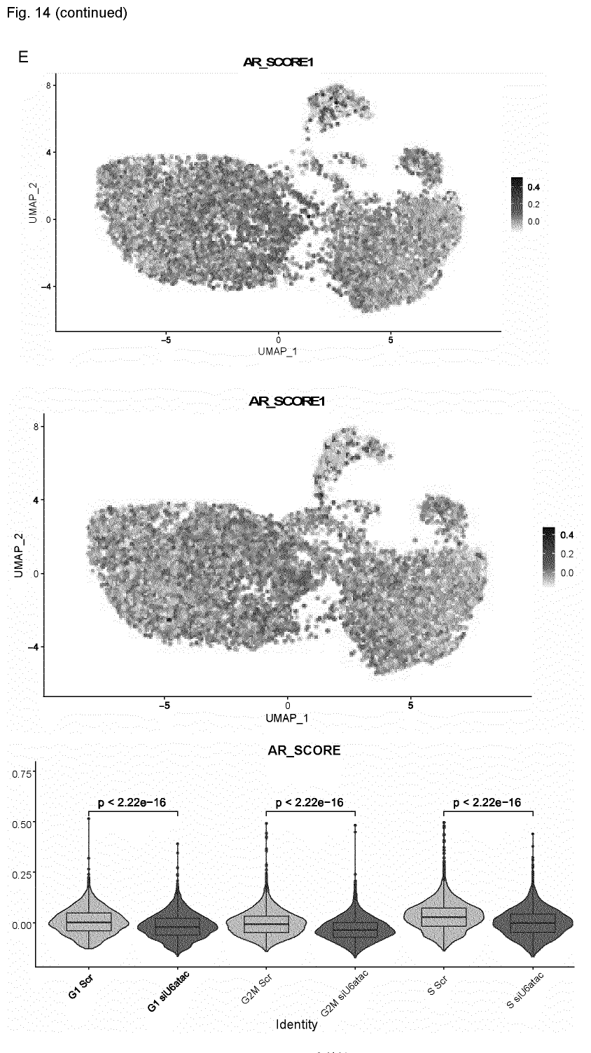

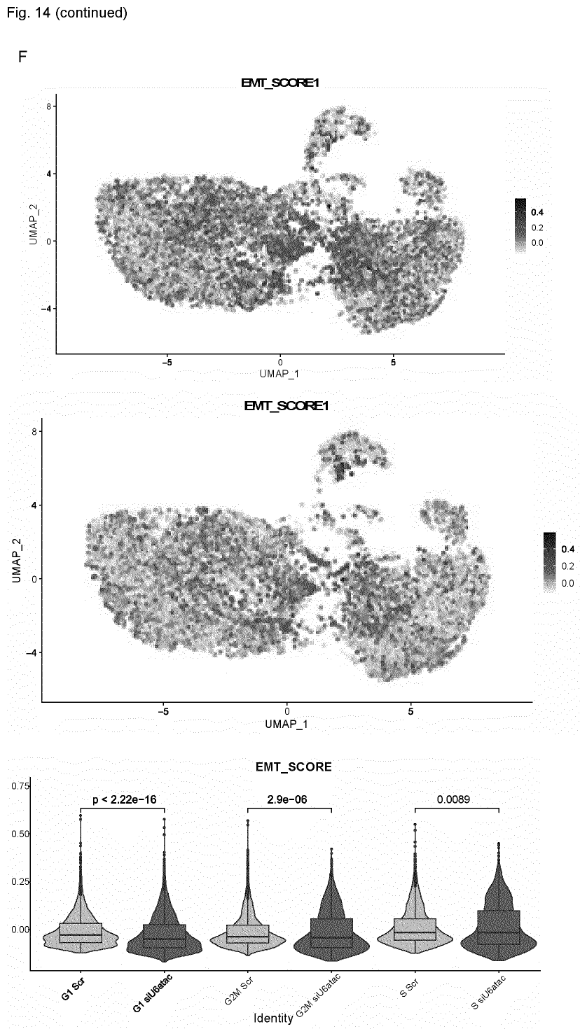

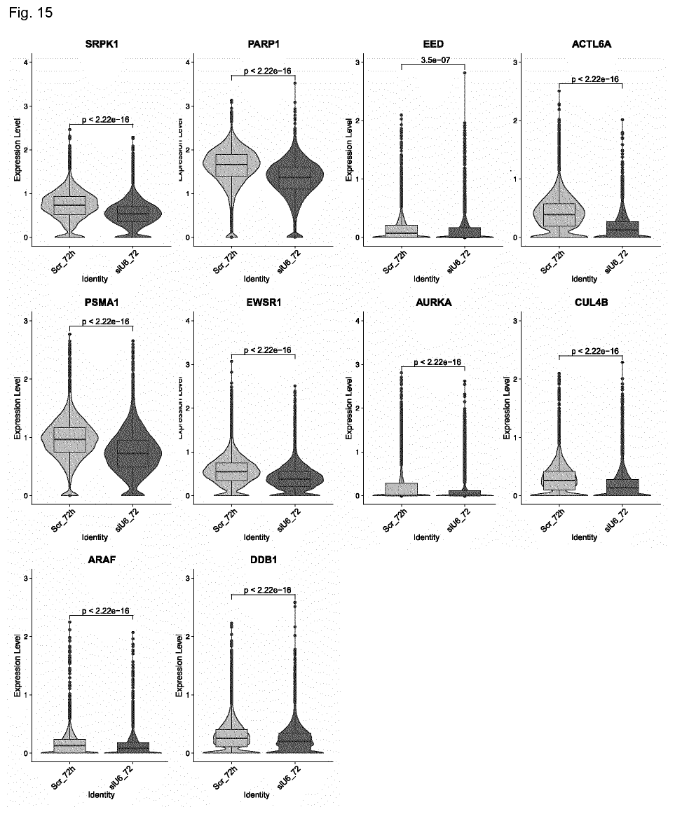

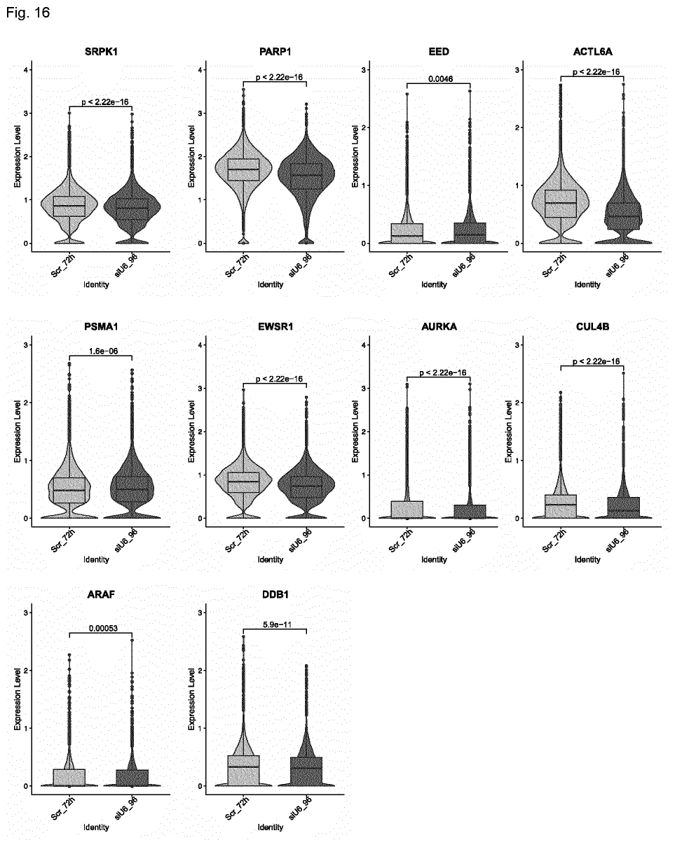

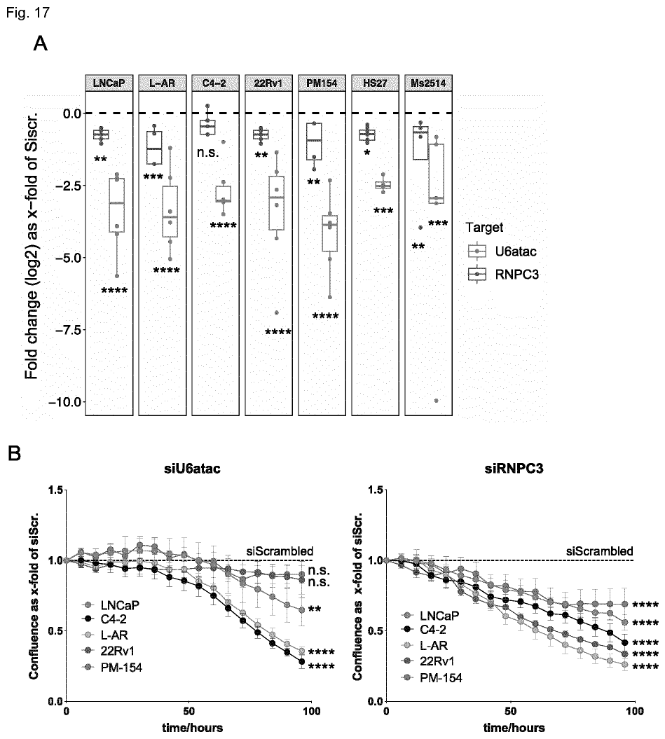

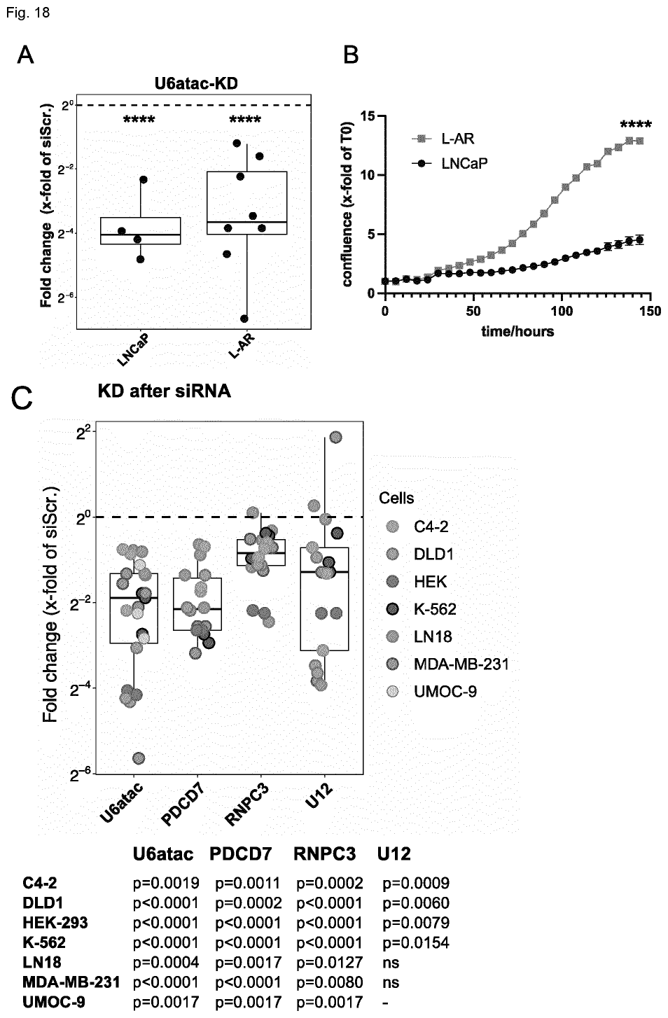

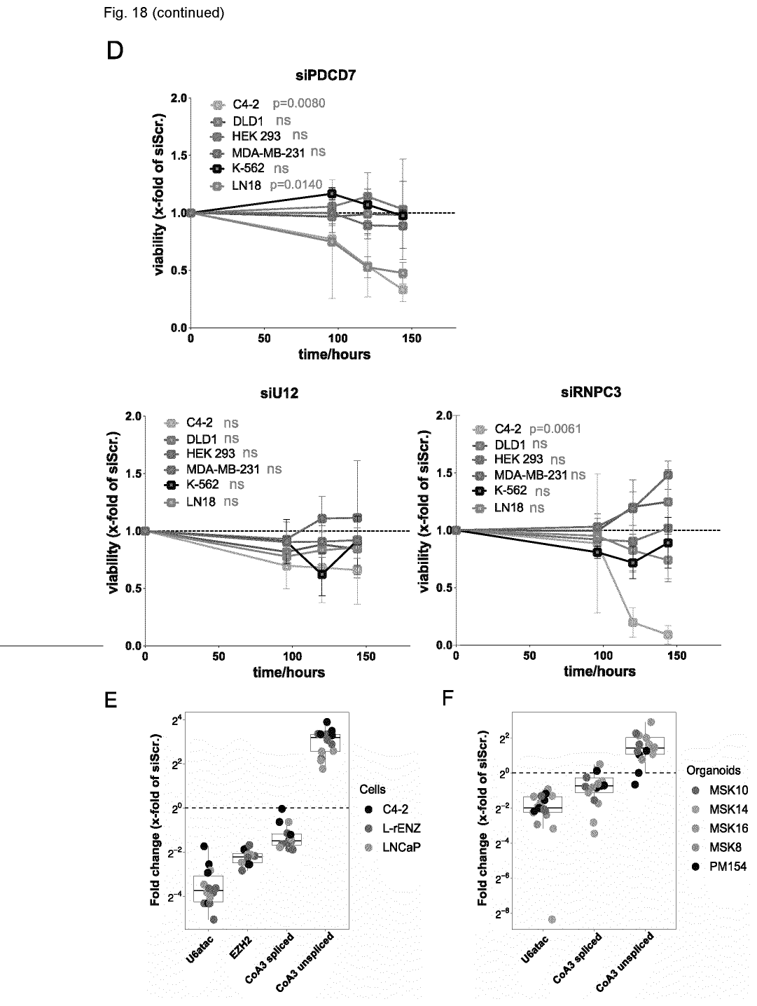

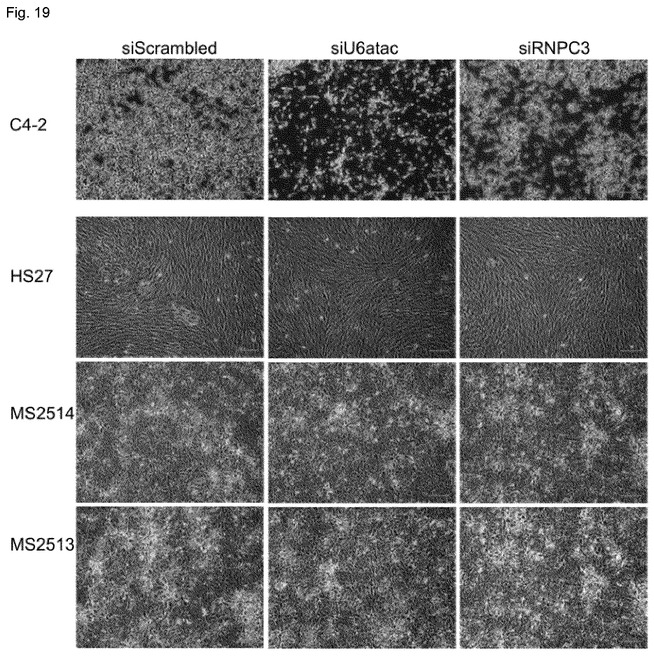

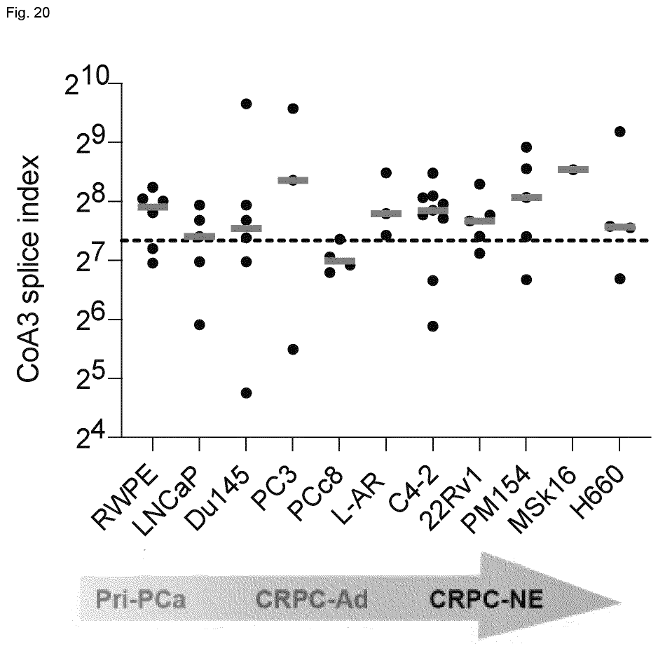

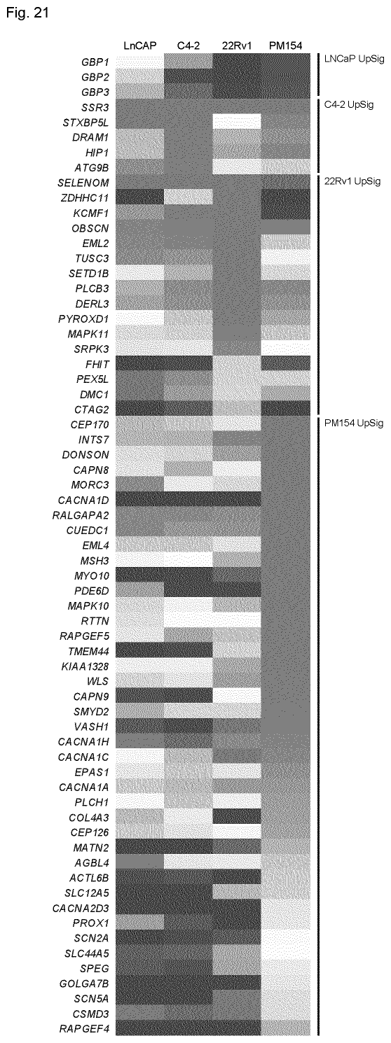

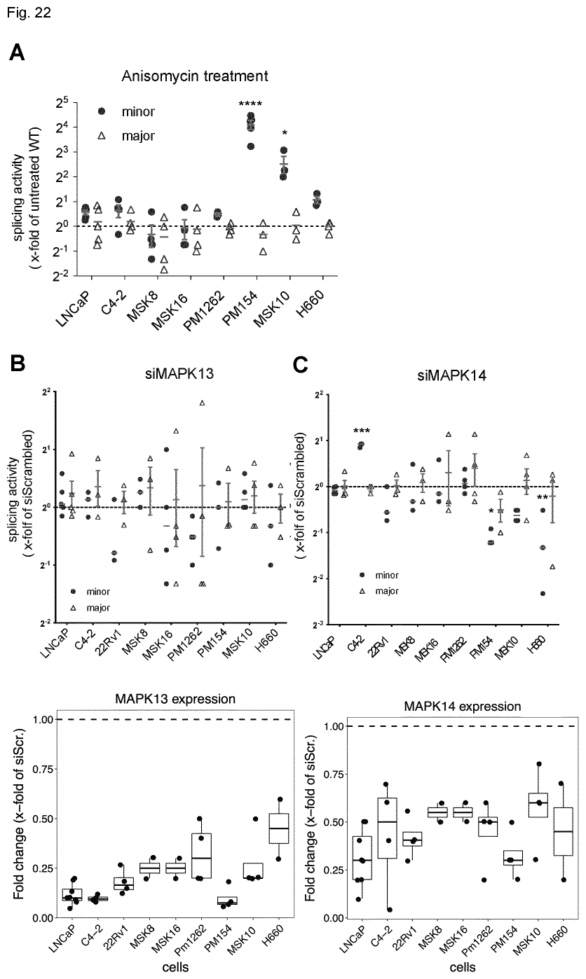

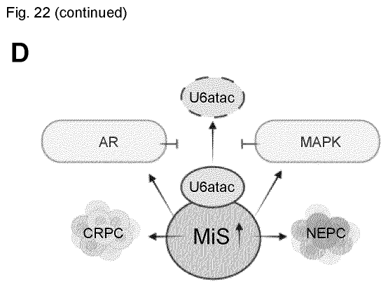

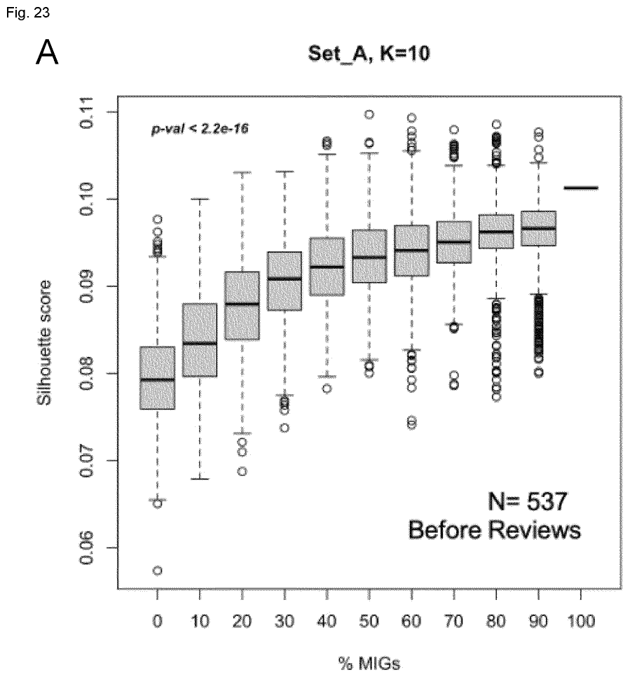

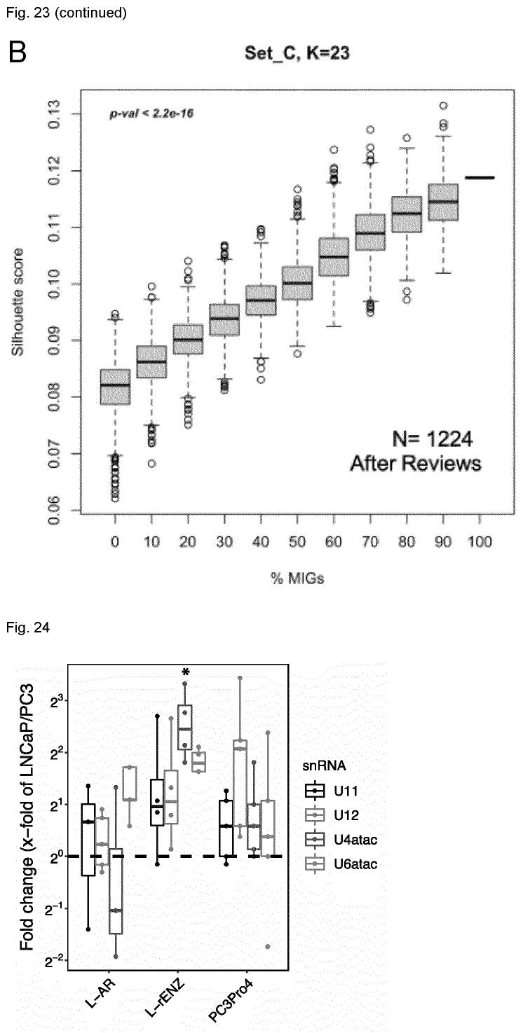

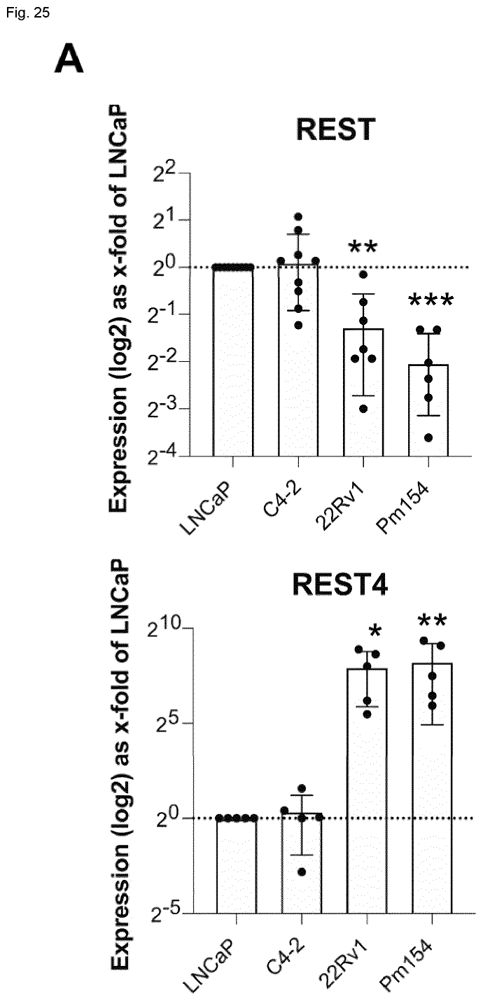

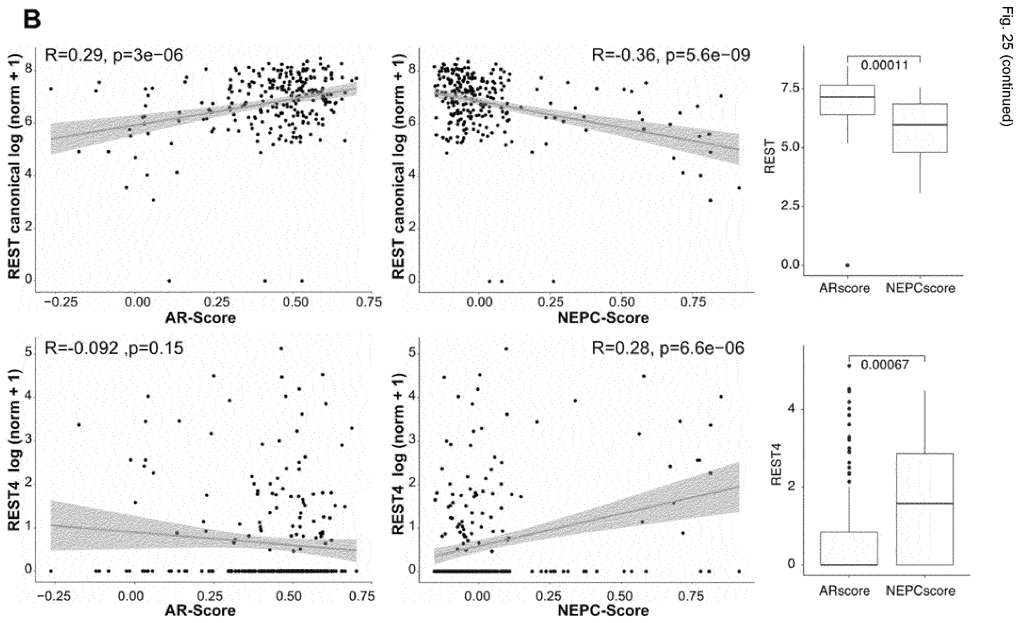

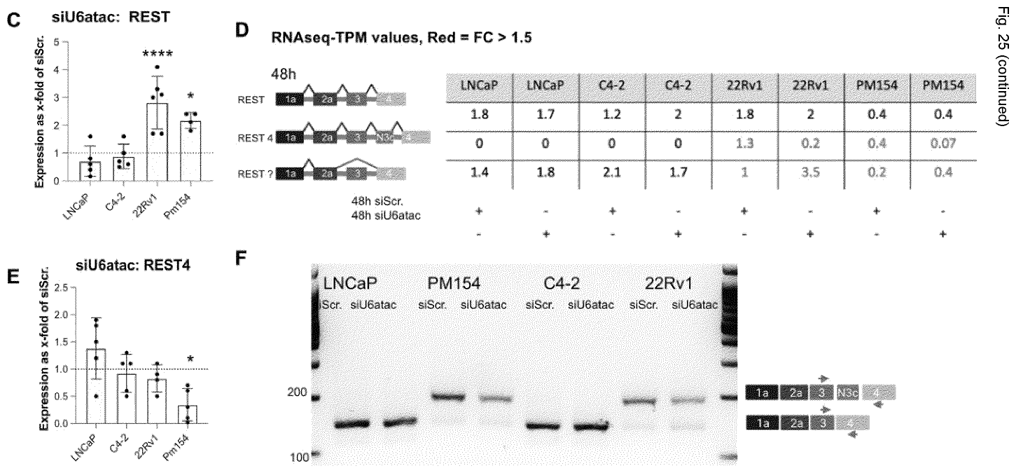

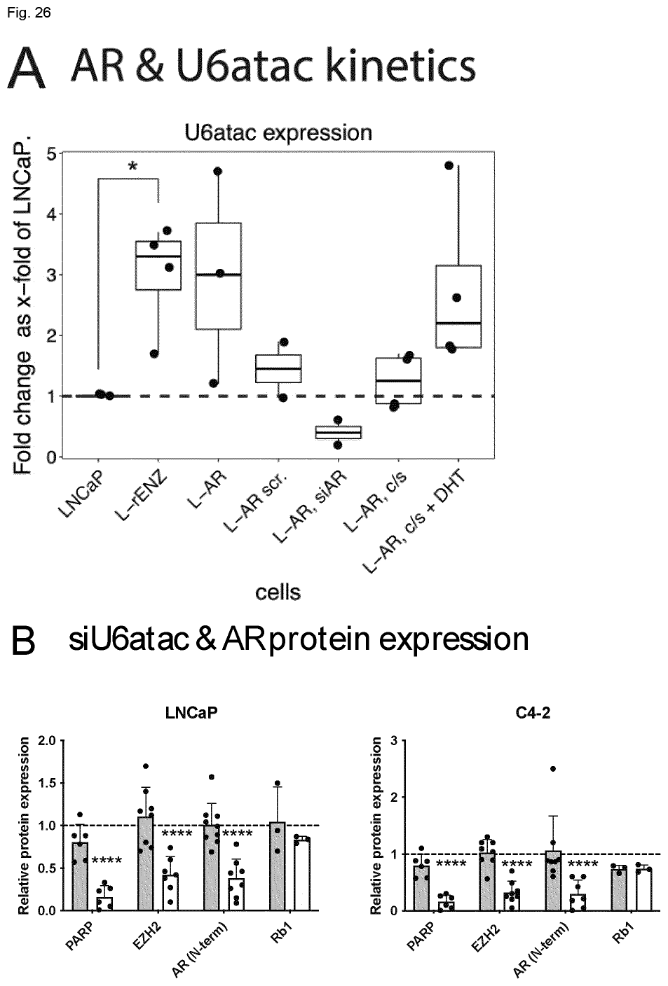

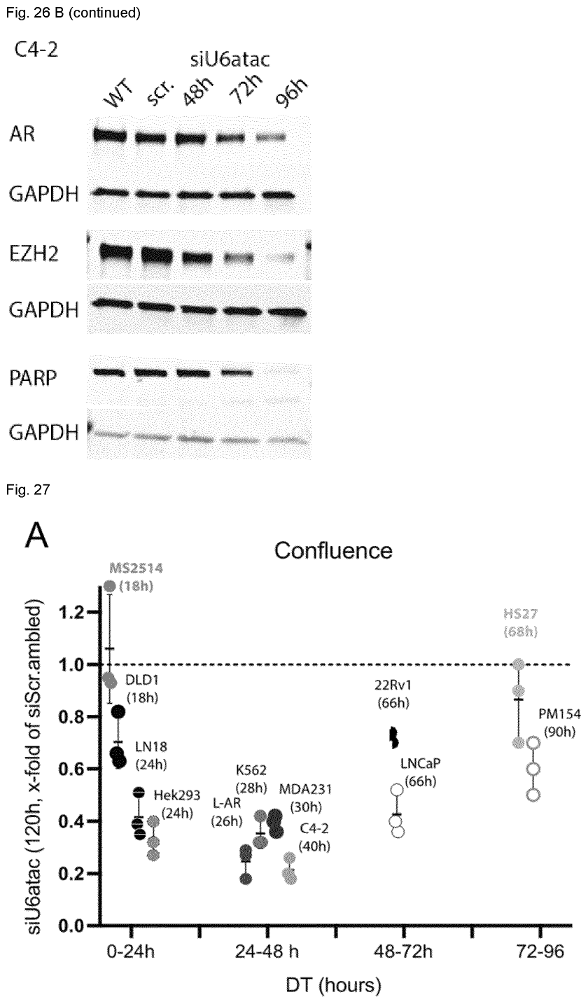

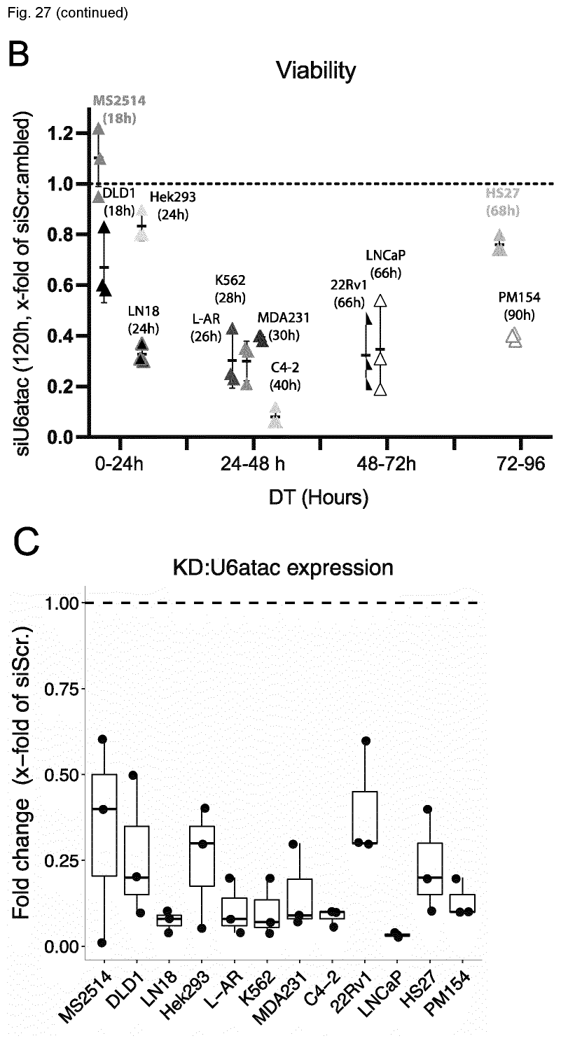

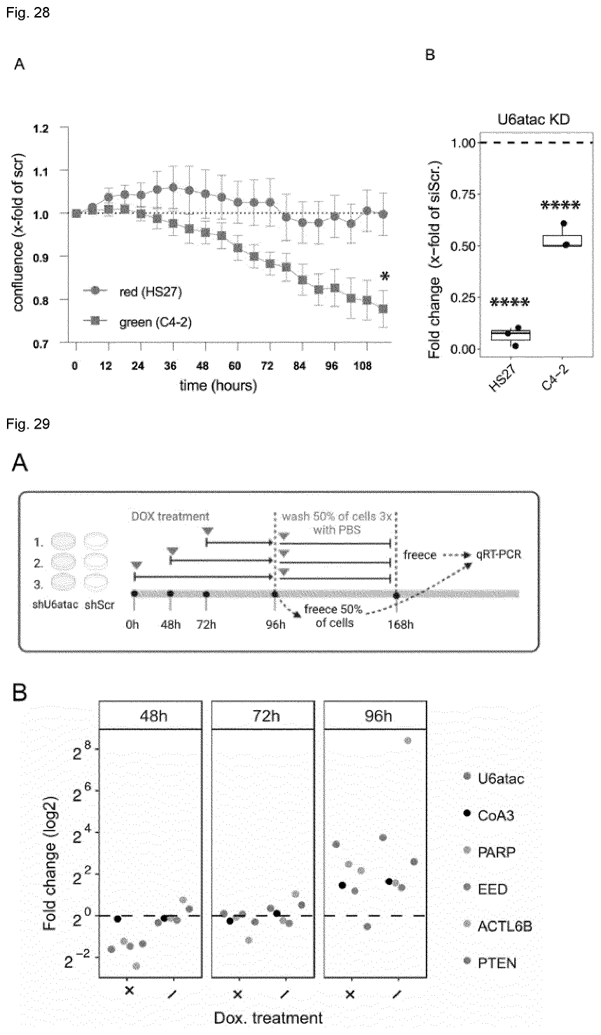

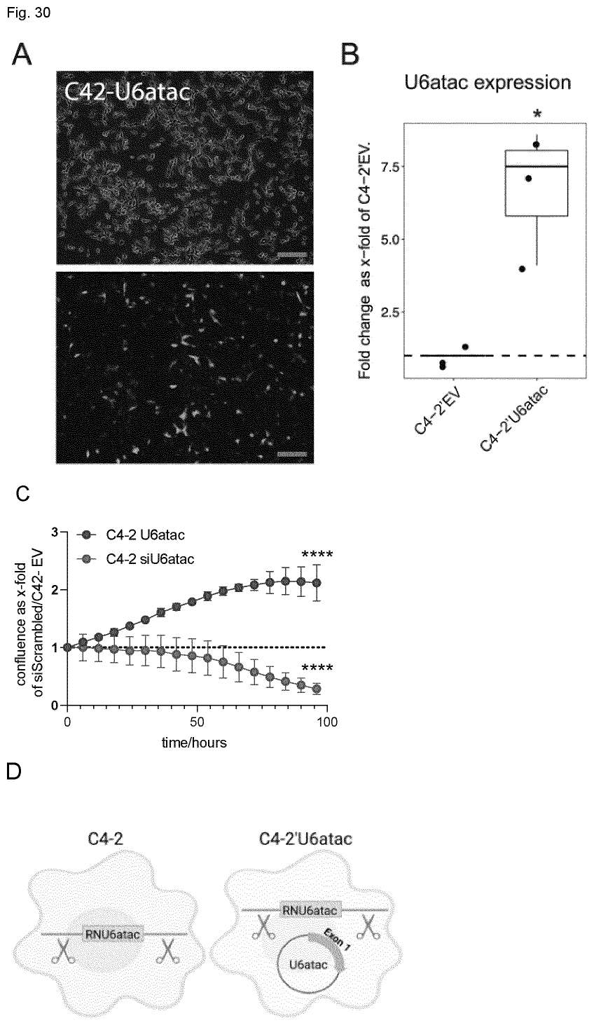

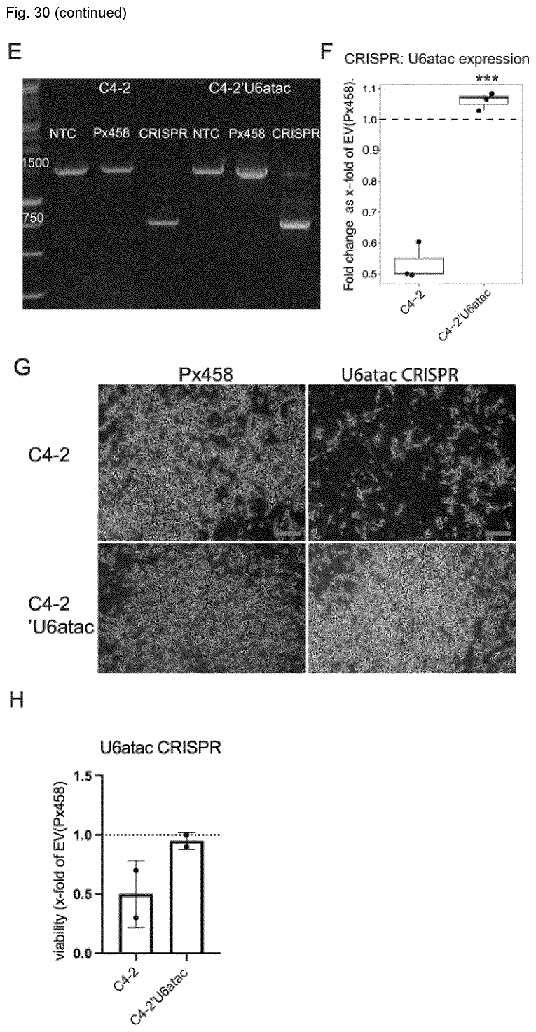

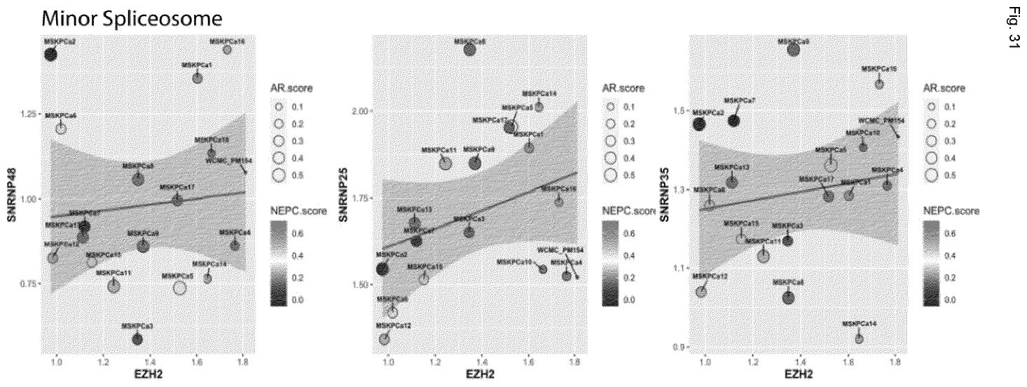

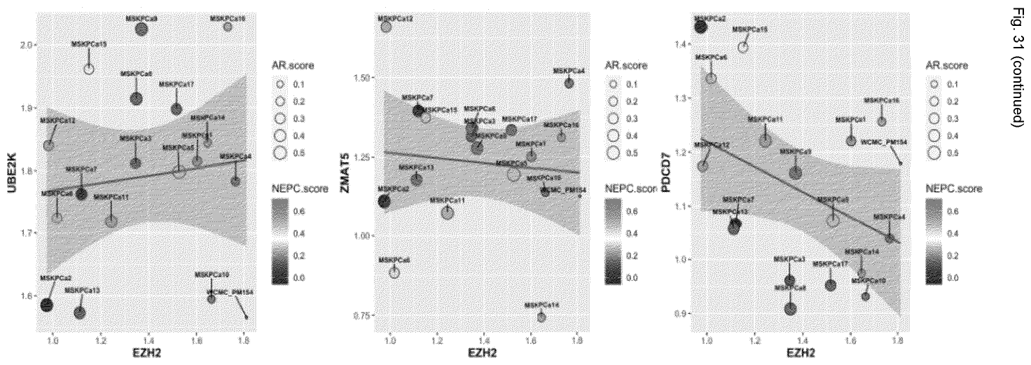

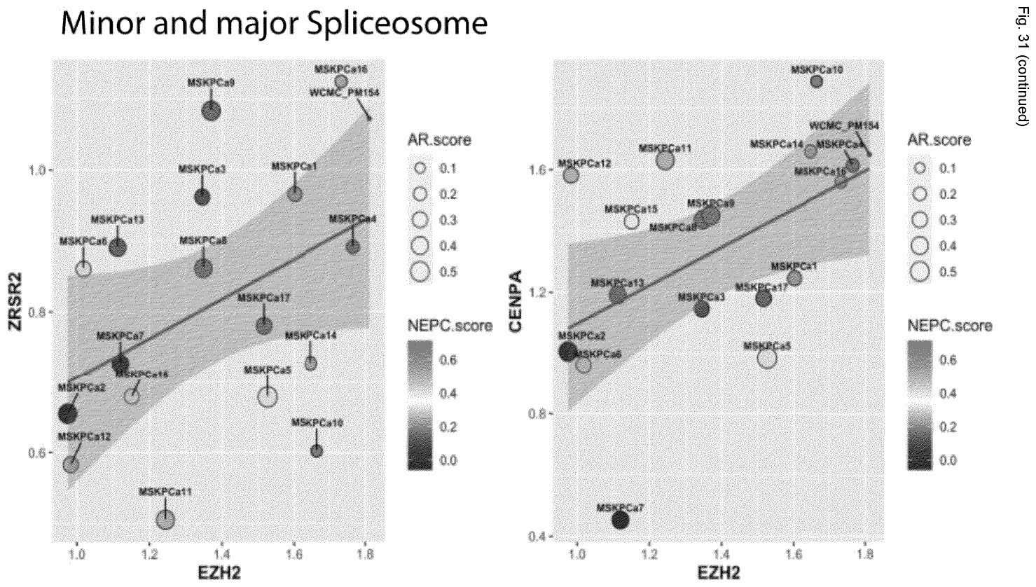

Based on the inventors' finding that the MiS plays a crucial role in PCa progression, the inventors next inhibited the MiS in PCa by targeting the U6atac snRNA, which normally exhibits higher rates of turnover but is detected at higher levels in advanced cancer stages. The inventors used siRNA against siU6atac in four PCa cell lines: LNCaP (primary PCa, therapy-sensitive), C4-2 (CRPC-adeno, therapy-resistant), and 22RV1 (CRPC-adeno, therapy-resistant) cell lines and a patient-derived organoid, PM154 (CRPC-NE, therapy-resistant). First, the inventors chose C4-2 cells to establish the kinetics of U6atac downregulation (FIG. 3A). Successful downregulation (˜7-fold, unpaired t-test: p<0.0001) of U6atac snRNA in C4-2 cells upon treatment with siU6atac was confirmed by qRT-PCR, in line with an increase in U6atac expression (˜6-fold, t-test: p<0.0001) in C4-2 cells overexpressing U6atac (FIG. 3A). The inventors then explored the kinetics of MiS inhibition on minor intron splicing by analysing COA3, which has a single intron that is a minor intron. The resulting minor spliceosome inhibition was reflected in a progressive increase of unspliced COA3 natural minor intron reporter transcripts across time, with the highest change observed at 96 hours post transfection, the latest time point investigated (FIG. 3B). Similar results were obtained in other cell lines (FIG. 10A). This finding was further confirmed by decreased splicing of three minor intron splicing minigene reporters p120, hSCN8A and hSCN4A (FIG. 10B) in siU6atac-treated cells compared to siScrambled controls. Additionally, failure to splice minor introns was observed by the decrease in luminescence signal with a luc-minor splicing reporter (FIG. 10C).

To capture a comprehensive effect of MiS inhibition, the inventors performed ribo-depleted total RNAseq on the four cell types treated with siU6atac and siScrambled for 96 hours. Equivalent levels of U6atac KD were observed in all 4 cell lines (FIG. 10D). The inventors first determined the overall level of minor intron retention by quantifying a mis-splicing index for each cell line. Comparison of the median mis-splicing index for each sample revealed significantly elevated minor intron retention in siU6atac-treated LNCaP, C4-2, 22RV1 cells, and PM154 compared their respective scrambled siRNA control (FIG. 3C) (Kruskal-Wallis test followed by post-hoc Dunn's test, *p<0.05, **p<0.01, ***p<0.001, respectively). In agreement with the inventors' previous report of cell type specific effects of MiS activity (FIGS. 1 and 2), the inventors found that the levels of minor intron retention in siU6atac-treated C4-2 cells was significantly greater than both siU6atac-treated PM154 and LNCaP cells (Kruskal-Wallis test and post-hoc Dunn's test: C4-2 vs LNCaP p<0.0001; C4-2 vs PM154 p=0.0296). Similarly, the level of minor intron retention in siU6atac-treated 22Rv1 cells was also significantly greater than siU6atac-treated LNCaP cells (Kruskal-Wallis test and post-hoc Dunn's test: p=0.0001). Since MiS inhibition is also predicted to trigger alternative splicing (AS) across minor introns (in addition to elevated minor intron retention), the inventors next quantified the number of AS-events detected in all eight samples. Indeed, the inventors found a robust increase in the number of AS-events occurring around minor introns in all four siU6atac-treated samples, with the most abundant change occurring for cryptic splice site usage. Regarding both minor intron retention and AS around minor introns, the largest effect upon siU6atac treatment was observed in C4-2 cells, followed by 22RV1 cells, then PM154, and lastly LNCaP cells (FIG. 3D).

Next, the inventors explored whether there was a cell-type specific effect of MiS inhibition by performing an intersection analysis of the MIGs with significantly elevated minor intron retention or AS. Here, the inventors separated the three cell lines from the organoid due to their different origins, culture conditions, genomic architectures and PCa phenotypes (CRPC-NE), which was also reflected in principal component analysis. Of the 380, 419, and 390 MIGs with significantly elevated minor intron retention in LNCaP, C4-2, and 22RV1 cells, respectively, the inventors found that 337 MIGs were common to all (FIG. 3E). The PM154 organoid was found to have 379 MIGs with elevated minor intron retention, of which 303 MIGs were shared with the three cell lines. GO-term enrichment analysis of each set of MIGs with elevated minor intron retention enriched for basic molecular pathways such as RNA and DNA processing, vesicle transport and mRNA splicing. Similarly, of the 159, 255, and 225 MIGs with significantly elevated AS events in LNCaP, C4-2, and 22RV1 cells, respectively, and 137 MIGs were common to all (FIG. 3F). In PM154 cells, the inventors detected 196 MIGs with significantly elevated AS-events (student's two-tailed T-test, p<0.05), of which 111 were shared with the three cell lines.

The aberrant minor intron splicing in siU6atac-treated samples should impact the overall transcriptome of PCa, which the inventors captured by differential gene expression analysis. The inventors set a 1 transcript per million (TPM) threshold for gene expression and, using isoDE2, the inventors found 68 genes that were significantly upregulated (log 2FC≥1, p≤0.01) and 691 genes significantly downregulated (log 2FC≤−1, p:s 0.01) in the siU6atac-treated LNCaP cells compared to siScrambled (FIG. 11). Similarly, the inventors found 154, 228, and 121 genes upregulated in 96 h siU6atac-treated C4-2, 22Rv1, and PM154 organoids, respectively, with 787, 638, and 189 genes downregulated, respectively (FIG. 11). Intersection analysis of the LNCaP, C4-2, and 22Rv1 cells revealed 11 upregulated protein-coding genes common to the three cell lines, and only 1 (CNST) was a MIG. In contrast, the inventors found 268 downregulated protein coding genes common to the three cell lines, and 18 were MIGs. Using DAVID, the inventors discovered that the shared downregulated genes enriched for many gene ontology (GO) Terms related to the cell cycle. Alternatively, the 189 downregulated genes in 96 h siU6atac-treated PM154 cells enriched for a single GO Term—cell differentiation—which was unique to this cell line. Gene Set Enrichment Analysis (GSEA) based on hallmark gene-sets on the up- and downregulated genes confirmed that pro-proliferative (E2F targets, G2M checkpoint, mitotic spindle) and DNA repair pathways were reduced upon siU6atac mediated MiS inhibition and additionally revealed a reduction in prostate-specific pathways such as androgen response or Spermatogenesis.

Next, the inventors sought to untangle the downstream molecular defect of aberrant minor intron splicing in conjunction with the transcriptomic changes captured by RNAseq. For this, the inventors took the list of MIGs with elevated minor intron retention common to either the LNCaP, C4-2, and 22Rv1 cell lines (FIG. 3G) and selected MIGs the inventors had identified as direct interactors of prostate cancer-causing genes (FIG. 1B and C). The inventors focused here on minor intron retention alone, and not MIGs with AS, as the molecular event is singular (the minor intron is either retained or not retained, whereas multiple AS events can occur in conjunction) and thus simplifies the downstream molecular predictions. This yielded 55 MIGs for the LNCaP, C4-2, and 22RV1 cell lines. The inventors hypothesized that minor intron retention in these MIGs would exert their influence on the transcriptome, which was reflected in the downregulated non-MIGs. Subsequently, the inventors investigated whether the MIGs and non-MIGs formed a PPI network by performing STRING network analysis on the combined list of 55 MIGs with minor intron retention plus 268 downregulated genes in all three cell lines. Most of the downregulated genes formed a tight-knit central network that was surrounded by MIGs with elevated minor intron retention (FIG. 3H). The same analysis for PM154 organoids showed a slightly more diffused network, though the MIGs with elevated retention were found to be clearly associated with an abundance of the downregulated genes (FIG. 3H). Subsequently, the inventors used Ingenuity Pathway Analysis (IPA) to i) uncover the biological pathways underlying these networks, and ii) determine if the same pathways affected in the cell lines were also affected in the organoid (FIG. 3). Of the 71 and 212 significantly enriched biological pathways for the cell lines and organoid, respectively, the inventors found 52 were shared. The overlapping pathways included terms such as cyclins and cell cycle regulation, PCa signalling, and MAPK signalling (FIG. 3). Notably, both the cell lines and the organoid enriched for numerous unique terms relevant to apoptosis and MAPK signaling. Finally, the inventors sought to breakdown the large STRING network into functionally relevant sub-networks, and then determine if similar sub-networks were affected in the cell lines and the organoid. The inventors found that only a single sub-network was shared in both conditions, where the three leading disease associations were cancer, haematological disease, and immunological disease (FIG. 3). All remaining sub-networks for the cell lines and the organoid did not overlap. Importantly, the sub-networks found to be enriched in the three cell lines were often associated with cell cycle (e.g., G2/M DNA Damage Checkpoint Regulation and G1/S Checkpoint Regulation) and DDR (e.g., Role of BRCA1 in DNA Damage Response and DNA Double-Strand Break Repair by Homologous Recombination). In contrast the organoid sub-networks were frequently associated with oncogenic signaling pathways (e.g., PI3K/Akt signaling and PTEN signaling) and with actions executed by the cell cytoskeleton (e.g., morphology changes, EMT and actin cytoskeleton), which regulates cancer hallmarks such as invasion, metastatic spread, migratory ability and cell movement. MIGs with elevated minor intron retention were found to be involved in nearly all the sub-networks for both the cell lines and the organoid. Overall, siRNA-mediated downregulation of U6atac resulted in a robust minor intron splicing defect in a large subset of MIGs involved in cancer-relevant pathways.

### Example 5: Impact of MiS Inhibition on the Prostate Cancer Proteome

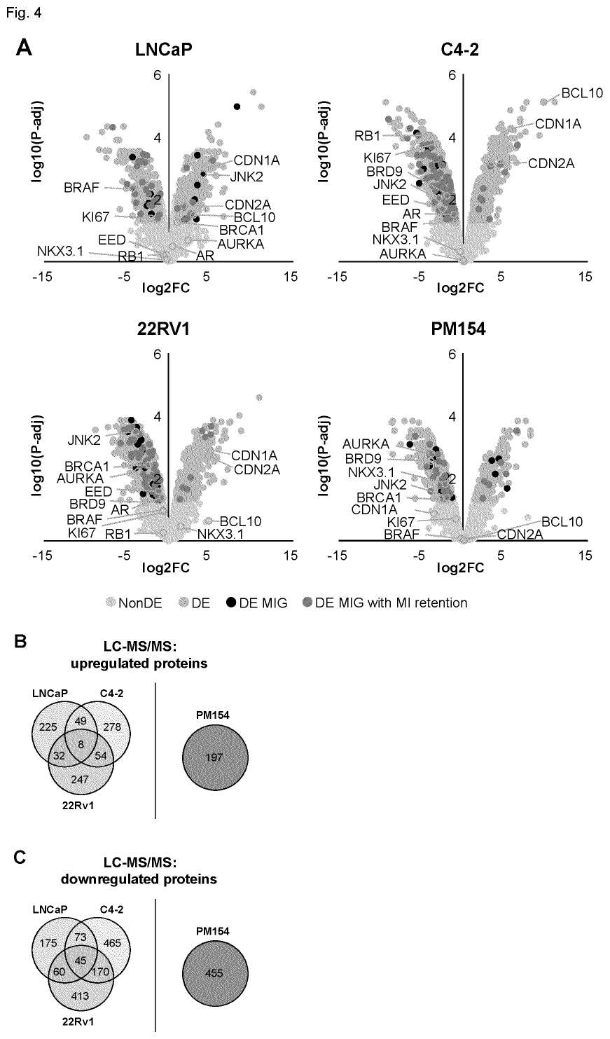

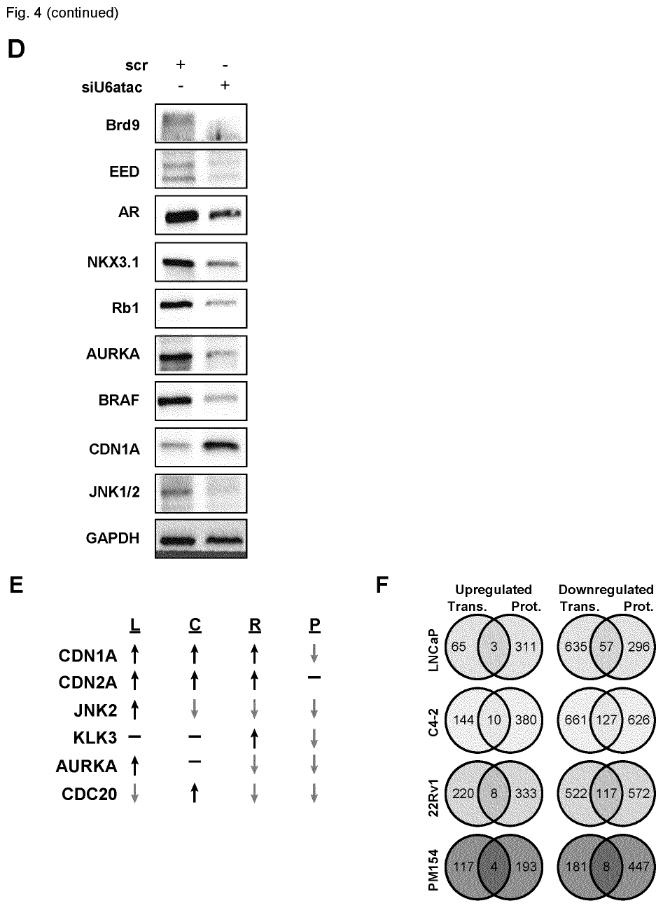

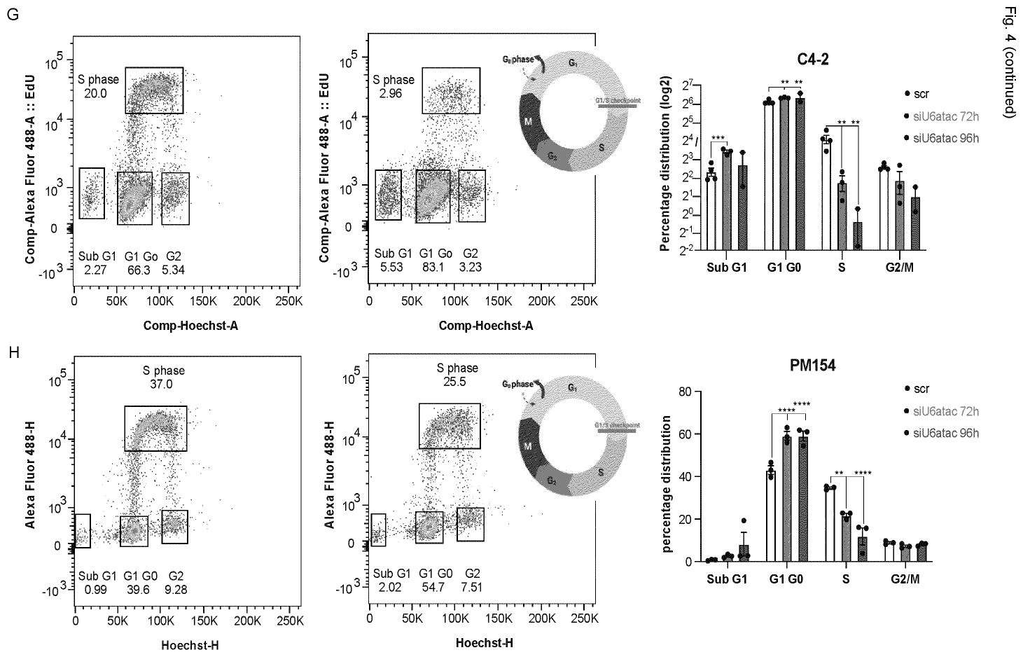

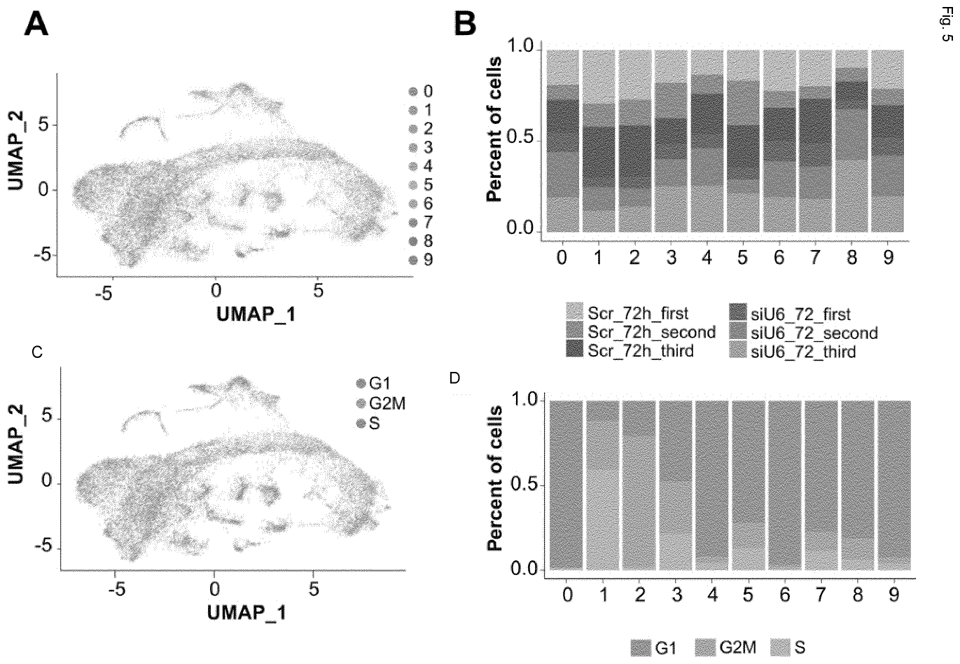

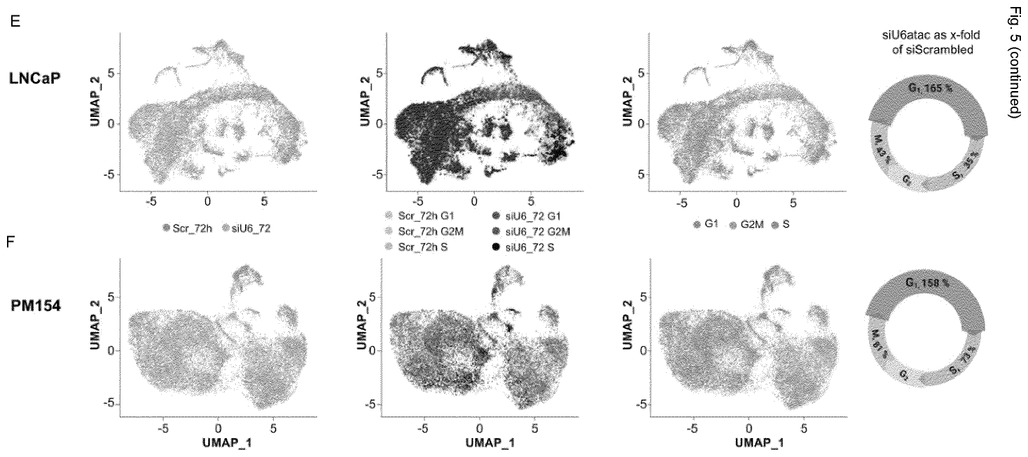

To determine whether the transcriptional changes captured for each cell line extended to differential protein production, the inventors performed LC-MS/MS on the same cell lines treated with siRNA (FIG. 4 and FIG. 12). With U6atac knock-down (log 2FC>±1, p≤0.05) the inventors found 314 up- and 353 downregulated proteins in LNCaP cells; 390 up- and 753 downregulated proteins in C4-2 cells; 341 up- and 689 downregulated proteins in 22RV1 cells; and 197 up- and 455 downregulated proteins in PM154 cells (FIG. 4A-E). For all cells, the inventors found that most differentially expressed MIG-encoded proteins were downregulated (FIG. 4A). Importantly, almost all MIGs with significantly elevated minor intron retention were found to be downregulated in the LC-MS/MS (FIG. 4A). For the three cell lines (LNCaP, C4-2, and 22Rv1), the inventors found shared upregulation of only 8 proteins (FIG. 4B), whereas 45 proteins were downregulated in all three (FIG. 4C). While none of the common upregulated proteins were encoded by MIGs, 9 of the common downregulated proteins were found to be MIGs, including SLC12A7, CEP170, TCEA3, POLA2, XPO4, WDFY1, ORC3, C17orf75, and SRPK1. Besides CEP170 and TCEA3 proteins, the rest of the proteins had significantly elevated minor intron retention in their respective transcripts by RNAseq in all cell lines. More importantly, POLA2 and SRPK1 are MIG-encoded proteins that directly interact with the proteins encoded by PCa-causing genes identified in FIG. 1C.

Comparing the proteomic data of the four analysed cell lines representing PCa disease progression revealed a cell type, and thus probably PCa subtype and context dependent MiS-dependent proteome, much like the RNAseq analysis. Each cell line expressed a unique set of up and downregulated proteins including MIGs and non-MIGs, (FIG. 4B and E) each in its own way important for cancer biology. Among others, the AR (non-MIG), Rb1 (non-MIG), the epigenetic regulator EED (MIG), a common target during PCa therapy, and the proliferation marker Ki67 (non-MIG) were strongly decreased upon siU6atac treatment in the CRPC-adeno line C4-2 (FIG. 4D). Similarly, the DNA damage response (DDR) proteins BRCA1 and AURKA (non-MIG) and JNK2 were decreased in therapy-resistant C4-2, 22Rv1 and PM154 lines but increased in therapy-responsive LNCaP cells (FIG. 4E). AURKA has been linked to lineage plasticity and neuroendocrine differentiation in PCa. Consistent with previous results, the inventors observed an increase in tumour suppressors such as the cell cycle regulators CDN1A (p21) (non-MIG) and CDN2A (non-MIG) in all three cell lines tested. in contrast, the inventors found that these proteins were decreased in the siU6atac-treated neuroendocrine organoid PM154 (FIG. 4E). Finally, the inventors also observed an increase in pro-apoptotic proteins, such as BCL10 (non-MIG), upon MiS inhibition. GSEA based on hallmark gene-sets on the up- and downregulated proteins confirmed that pro-proliferative (E2F targets, G2M checkpoint, mitotic spindle) and DNA repair pathways were reduced, whereas apoptotic and stress sensing pathways (p53, unfolded protein response, UV response or hypoxia mediated oxidative stress) were increased upon siU6atac mediated MiS inhibition. Differential protein expression was validated by WB analysis (FIG. 4D and FIG. 13).

The inventors next explored concordance between the transcriptome and proteome data, which were generally discordant in each cell line (FIG. 4F). MIGs with elevated retention upon MiS inhibition have been reported to escape NMD. For example, the inventors found proteins such as EED, JNK1/2, BRAF and RAF1 to be decreased even though their expression by RNAseq remained unchanged, albeit with elevated minor intron retention and AS upon siU6atac treatment (FIG. 4A-E). The inventors also found proteins such as the AR and Rb1 to be decreased only in the proteome, implying indirect regulation mechanisms executed through the MiS. Despite this, the inventors identified 57, 127, 117, and 8 genes whose mRNA transcripts and encoded proteins were significantly downregulated for 96 h siU6atac-treated LNCaP, C4-2, 22Rv1, cells and PM154 organoids, respectively (FIG. 4F). For example, the inventors found the proliferation marker Ki67 not only to be downregulated in the transcriptome but also in the proteome of LNCaP and C4-2 cells. 22Rv1 cells showed a decrease in the DDR proteins BRCA1 and AURKA/B. In contrast, CDN1A (p21) was upregulated in transcriptome and proteome in all three cell lines. Upon independent submission of these four gene sets to DAVID, the inventors found a high degree of GO Term overlap that mostly centered on cell cycle. For example, genes with similarly downregulated transcripts and encoded proteins in LNCaP, C4-2, and 22RV1 cells enriched for cell division, mitotic nuclear division, spindle pole, sister chromatid cohesion, G2/M transition of mitotic cell cycle, cell proliferation, and DNA strand elongation involved in DNA replication. Similarly, the 8 genes downregulated both transcriptionally and proteomically in the siU6atac-treated PM154 organoid enriched for a single GO Term—mitotic metaphase plate congression—which was also enriched by the siU6atac-treated 22RV1 cells. Taken together, siU6atac-mediated MiS inhibition altered the transcriptome and the proteome, which together enriched for cell cycle regulators. Based on the molecular signature, the inventors next explored whether MiS inhibition would indeed affect cell cycle progression. Therefore, the inventors employed FACS analysis at 72 and 96 hours post transfection, which revealed a significant increase (two-way ANOVA, 72 h: **p=0.0027, 96 h: **p=0.0018) in G1/GO phase cells and a significant decrease (two-way ANOVA, 72 h**p=0.0080, 96 h: **p=0.0085) in S-phase cells in therapy-resistant C4-2 cells treated with siU6atac (FIG. 4H). PM154 organoids showed a similar result under siU6atac conditions (FIG. 41). These cell cycle defects observed here revealed that MiS inhibition provokes a G1/S cell cycle arrest in PCa.

### Example 6: Single Cell RNAseq Reveals Cell Cycle Defects Triggered by MiS Inhibition

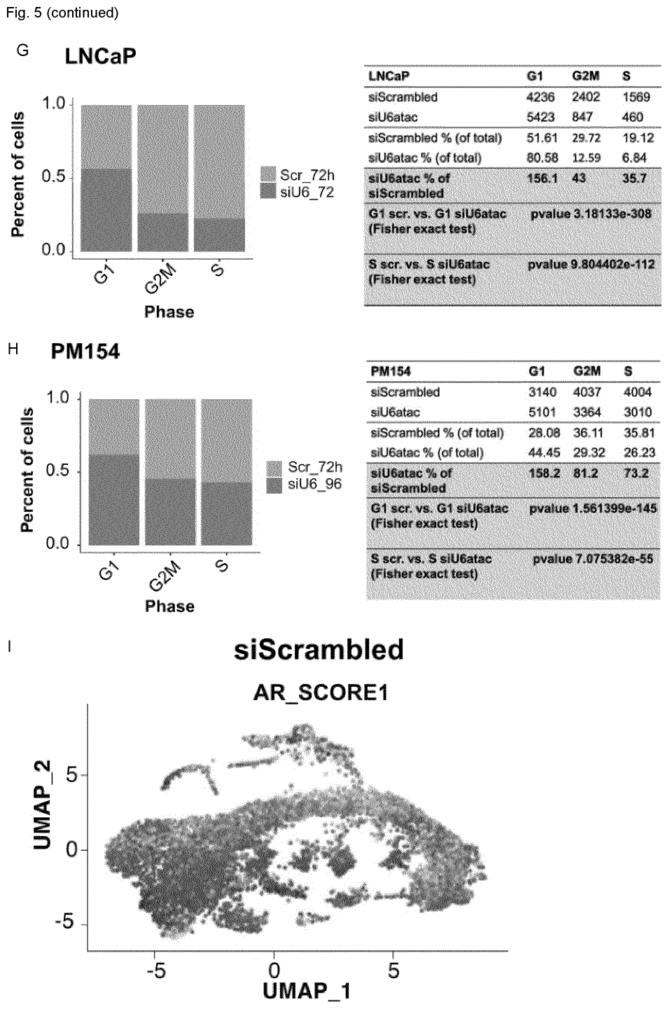

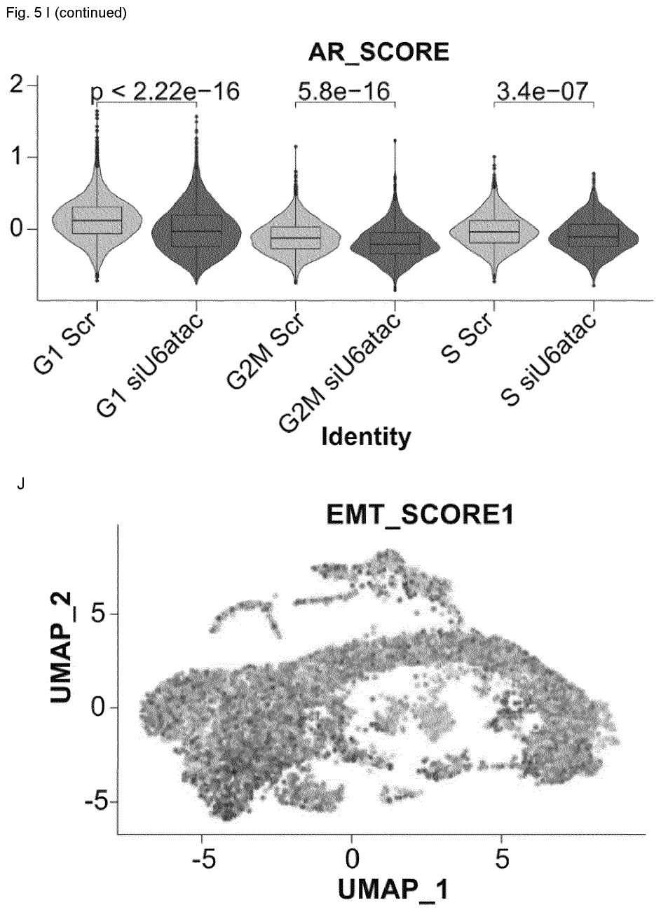

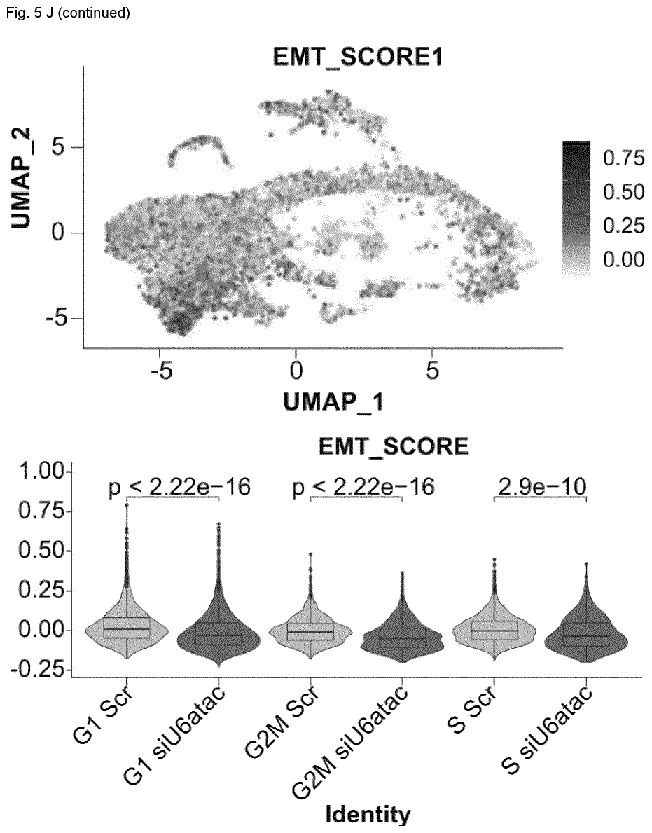

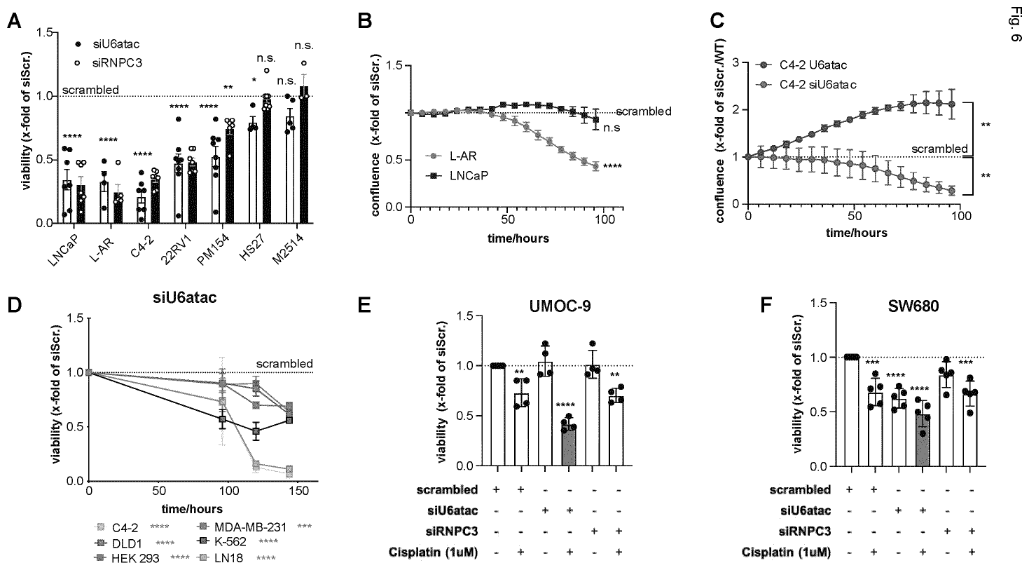

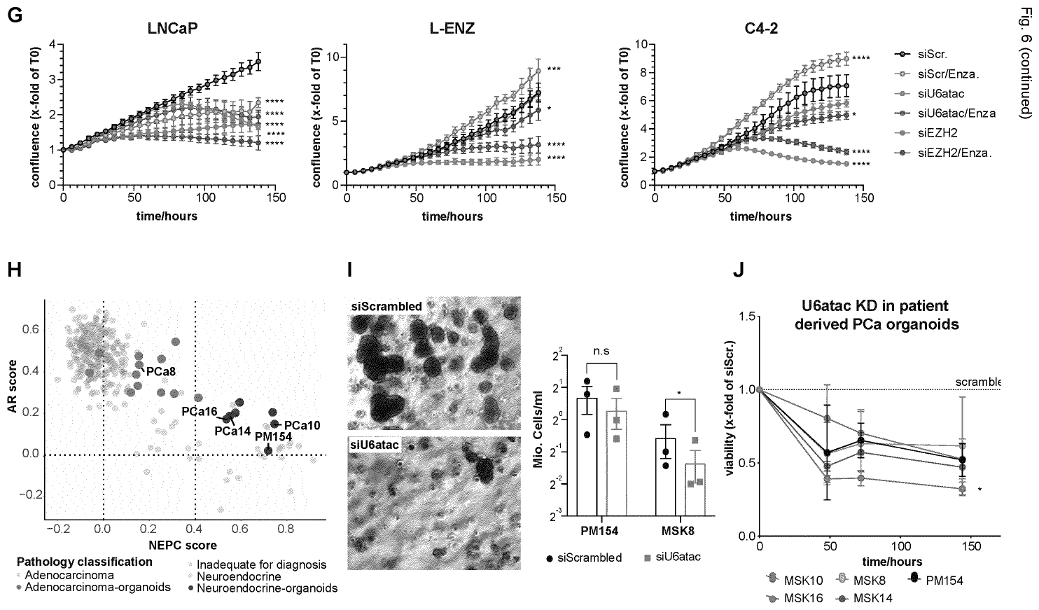

Next, the inventors wanted to identify cellular heterogeneity in the response to siU6atac mediated MiS inhibition. The inventors thus also performed single cell RNAseq (scRNAseq) on LNCaP cells and PM154 organoids post siU6atac. After standard data processing and quality control procedures (methods) the inventors obtained transcriptomic profiles for 8206 siScrambled and 6730 LNCaP cells and 11181 siScrambled and 11475 PM154 organoids. The inventors employed unsupervised clustering to identify heterogeneity in response to siU6atac. K-means clustering and UMAP projection of the combined data across both genotypes, which revealed 9 major cell clusters (FIG. 5A). The inventors found that clusters 0, 3, 4, 6, 7 and 9 were populated by an equal number of cells treated with siScrambled and siU6atac (FIG. 5B). Clusters 1(Fisher's exact test, p=3.16934e-69), 2 (Fisher's exact test, p=1.702664e-63) and 5 (Fisher's exact test, p=3.977987e-05) showed higher percentages of siScrambled cells, relative to siU6atac cells. Finally, cluster 8 (Fisher's exact test, p=1.533612e-14) was predominantly populated by cells treated with siU6atac compared to siScrambled (FIG. 5B). GO Term enrichment analysis of the Top 5 upregulated genes, in the cells belonging to clusters 1, 2 and 5 revealed cell division, G1/S transition and mitotic nuclear division. In contrast, there was no Go Term enrichment for the Top 5 genes in cluster 8. The enrichment of cell cycle terms combined with previous findings that MiS inhibition impacts cell cycle regulation in cancer (FIG. 3,4) led the inventors to employ a list of cell cycle regulators as a means of sorting single cells based on cell cycle stage. Thus, the inventors superimposed cell cycle stage on the UMAP representation of the unsupervised data clustering (FIG. 5D). The inventors found that clusters 0, 3, 4, 6, 7 and 9 showed the highest percentage of G1 cells (FIG. 5E), whereas clusters 1 and 2 showed the highest percentage of cells in S and G2/M phase respectively (FIG. 5E). Similar analysis for PM154 organoids yielded 7 clusters (FIG. 14A). A majority of the cells populating cluster 5 (Fisher's exact test, p=3.287745e-40) were from siScrambled, while cluster 6 (Fisher's exact test, 2.738288e-57) was predominated populated by siU6atac-treated cells (FIG. 14B). There was no Go Term enrichment for genes upregulated in those clusters, yet cluster 5, as with clusters 1, 2 and 5 of LNCaP cells, showed a high percentage of cells in S and G2/M phase, respectively (FIG. 14C, D).

The comparison of distribution of cells from each condition in the 9 clusters and the distribution of cells in the different phases of cell cycle led the inventors to investigate cell cycle defects through scRNAseq analysis (FIG. 5). Therefore, the inventors superimposed the cell identities (i.e., siScrambled or siU6atac) onto the UMAP of the clusters along with the cell cycle phases (FIG. 5). For LNCaP cells, the inventors found a significant enrichment (Fisher's exact test, p=3.18e-308, OR=3.89, 95% Cl 3.60-4.19) of siU6atac-treated cells in G1 phase and a reduction of these cells in S phase (Fisher's exact test, p=9.80e-112, OR=3.22, 95% Cl 2.88-3.60) (FIG. 5). Similarly, for PM154 organoids, the inventors observed a significant enrichment of siU6atac cells in G1 (Fisher's exact test, p=1.56e-145, OR=2.05, 95% CI 1.93-2.16) and a decrease in S-phase (Fisher's exact test, p=7.07e-55, OR=1.57, 95% CI 1.48-1.66) (FIG. 5). These findings are consistent with FACS analysis (FIG. 4).

Given that siU6atac is successful at blocking cell cycle progression in PCa and that AR signaling is a crucial driver of PCa, the inventors next explored the AR score (a crucial metric of CRPC-adeno progression) in siU6atac-treated LNCaP cells and PM154 organoids (FIG. 5 and FIG. 14E). The inventors discovered that siU6atac was indeed able to reduce the AR scores of both models significantly in G1 (Wilcoxon test, p<2.22e-16), G2M (Wilcoxon test, LNCaP: p=5.8e-16, PM154: p<2.22e-16) and S-phase (Wilcoxon test, LNCaP: p=3.4e-07, PM154: p<2.22e-16). The inventors did not observe any shift in the AR score post siU6atac treatment in bulk RNAseq (FIG. 14G), which is not surprising, as the inventors lose single cell resolution and dilution of expression of some of the key AR score markers. Another hallmark of cancer progression is EMT, which the inventors investigated by using an EMT score (Dong, B et al., Communications Biology 2020, 3 (1), 778). The inventors found that EMT score was also significantly reduced in G1 (Wilcoxon test, p<2.22e-16), G2M (Wilcoxon test, LNCaP: p=2.9e-10, PM154: p=2.9e-06) and S-phase (Wilcoxon test, LNCaP: p=3.4e-07, PM154: p=0.0089) in siU6atac-treated cells and organoids (FIG. 5 and FIG. 14F). Next, the inventors looked at the expression of MIGs that were identified as most crucial nodes from the inventors' PPI analysis in the scRNAseq data (FIG. 1B and C). Indeed, the inventors found a significantly lower number of siU6atac-treated cells and organoids that expressed these MIGs such as AURKA, EED, and PARP1 (FIG. 15 and FIG. 16).

Together, scRNAseq Revealed siU6atac Mediated Transcriptomic Remodeling that Contributes to PCa progression and lineage plasticity by decreasing cell cycle, AR signaling and EMT.

### Example 7: The Minor Spliceosome is Essential for PCa Growth and Viability

Application of siU6atac significantly decreased proliferation in hormone responsive LNCaP cells and in therapy-resistant L-AR, C4-2, 22RV1 and PM154 cell and organoids (FIG. 6A and FIG. 17A and B). Previous reports have shown tissue-specific minor intron splicing as reflected by differential rate of minor intron retention across various tissues. Thus, the inventors explored whether siU6atac treatment resulted in a similar, context dependent failure to survive. Indeed, the inventors observed a significant decrease in confluence of L-AR cells compared to LNCaP cells upon siU6atac treatment (FIG. 6B, FIG. 18A). L-AR cells showed a higher proliferation defect than LNCaP cells, most likely due to higher doubling rate of L-AR cells (FIG. 18B). L-AR cells (as well as therapy-resistant cells) were shown to possess higher MiS activity than their native hormone responsive lines. These data suggest that a higher basal MiS activity (FIG. 2) is indicative of an increased dependency on this non-canonical splicing mechanism. Of note, viability of non-cancer cells, such as fibroblasts (HS27) and primary mouse prostate cells (M2514), were not affected by siU6atac-mediated MiS inhibition (FIG. 6A and FIG. 17A and FIG. 19). In accordance with the KD data, overexpression of U6atac in C4-2 cells re-enforces cell growth (FIG. 6C), which agrees with previous findings showing that the MiS is essential for cell proliferation and plays a major role in cell cycle progression.

While the inventors targeted U6atac to inhibit the MiS, the minor spliceosome complex consists of other unique components, including snRNAs and snRNPs. Therefore, the inventors chose to inhibit the MiS by targeting RNPC3, another component of the MiS. Indeed, RNPC3 KD provoked similar reactions in PCa cells to siU6atac, indicating that the observed decrease in cell proliferation and viability can be attributed to a decrease in MiS activity in general and is not a U6atac-specific observation (FIG. 6A and FIG. 17A and B). Importantly, the inventors found that MiS inhibition not only affects PCa, but that the viability of other cancer cell lines such as colon, kidney, breast, glioblastoma, and CML was also decreased by a KD of several MiS components (FIG. 6D and FIG. 18C and D). This finding reveals that the MiS is essential for other cancer types and that dependency on its integrity might represent a broad phenomenon that is not restricted to PCa alone. Particularly, among all tested siRNAs (RNPC3, PDCD7, U12 and U6atac), a U6atac KD turned out to be the most efficient. Surprisingly, even KD of U6atac was not sufficient to decrease viability of the bladder cancer lines UMOC9 and SW680. However, siU6atac (in combination with cisplatin) led to a significant decrease in viability (FIG. 6E and F), suggesting that, in certain cases, MiS inhibition might work synergistically with current state-of-the-art treatments. In fact, CRPC requires combination therapy like inhibition of EZH2, which sensitizes towards ARSi (enzalutamide) treatment (Ku, S. Y. et al., Science 2017, 355 (6320), 78-83; Bai, Y. et al., J Biol Chem 2019, 294 (25), 9911-9923). Therefore, the inventors tested whether U6atac inhibition plus enzalutamide might be similar or better than EZH2 inhibition plus enzalutamide. The inventors observed that U6atac KD plus enzalutamide was more effective than EZH2 plus enzalutamide in CRPC C4-2 cells and in enzalutamide-resistant LNCaP (L-rENZ) cells (FIG. 6G and FIG. 18E). Surprisingly, U6atac KD alone was significantly better than EZH2 plus enzalutamide.

Finally, the inventors wanted to further explore the effects of MiS inhibition in a model system that captures PCa disease heterogeneity better than the previously used cell culture studies. Therefore, the inventors applied siRNA against U6atac in PCa patient-derived organoids that were cultured in 3D. Superimposing the RNAseq results of those organoids with clinical data from the SU2C study (FIG. 6H) confirmed the clinical relevance of those model systems. Treatment of the organoids MSK8 (PCa8, CRPC-Ad), MSK10, 16,14 (PCa 10,16,14, CRPC-NE) and PM154 (CRPC-NE) with siU6atac provoked a significant (two-way ANOVA, p=0.0246) reduction (Mskl6: P=0.019) in organoid growth and viability (FIG. 6I and J and FIG. 18F). Collectively, these data show that knocking down MiS components (especially U6atac) is sufficient to decrease cancer growth and viability.

In summary, the inventors conducted the first-in-field evaluation of MiS in PCa using a multi-pronged approach that combined RNAseq, mass spectrophotometry, FACS, and scRNAseq. This demonstrates that MiS inhibition results in severe cell cycle defects through aberrant splicing of MIGs, which fundamentally impacts PCa progression, survival and lineage plasticity

### Example 8: Minor Splicing Correlates with PCa Progression

The rational for the inventors' usage of reporter assays as splicing readout is that the assessment of splicing kinetics of endogenous genes is often complicated by multiple factors (chromatin architecture, rate of transcription, trans-acting factors and the activity of the nonsense mediated decay (NMD) pathway). In the field of splicing especially minor intron splicing there is therefore a long history of using splicing reporters like the one the inventors used in the current study. The rational especially when comparing splicing in different cell types or cancer stages is that the expression of the transfected splicing reporter is independent of the endogenous regulatory modalities. Thus, the same amount of expression of the reporter across different cell lines allows the inventors to truly interrogate the efficiency of MiS activity.

To assess the whether the changes seen by reporter assays reflect the changes of endogenous MIGs within each cell line. This question relates to the inventors' splicing kinetics analysis of CoA3 which the inventors performed to verify that siU6atac indeed impacts minor splicing of MIGs (FIGS. 10, 12, 18). CoA3 is one of the rare genes that has only one intron that happens to be a minor intron and thus represents a good endogenous MIG reporter of minor intron splicing. To address this the inventors have now measured expression of spliced CoA3 as x-fold of unspliced CoA3 (splice index) in most of the cell lines used in the manuscript (FIG. 9E, 24). Due to the above explained reasons, values of endogenous CoA3 splicing were highly variable yet the inventors do also observe a trend towards more advanced PCa lines showing a higher CoA3 splice index.

### Example 9: MIG Expression in LNCaP, C4-2, 22Rv1 and PM154 Cells

The inventors further analyzed the RNAseq datasets of the four cell lines representing different stages of PCa progression (LNCaP, C4-2, 22Rv1 and PM154). Specifically, the inventors analyzed the RNAseq data of the samples treated with scrambled siRNA which represents the de novo control transcriptome (FIGS. 21, 24). The inventors found that the number of MIGs expressed and therefore the amount of MiS requirement increased with PCa progression and androgen receptor signaling inhibition (ARSi) therapy resistance (LNCaP (3 MIGs)<C4-2 (5 MIGs)<22Rv1 (16 MIGs)<PM154 (41 MIGs)). Levels of endogenous expressed MIGs within each cell line thus resemble the inventors' reporter assay results in this analysis.

Interestingly the inventors observed that while therapy responsive LNCaPs expressed MIGs that play a role in anti-viral response, MIGs of C4-2 cells encompassed proteins involved in DNA damage and autophagy. MIGs of 22Rv1 included MAPK11 as well as tumor antigens (CTAG2) or splice factors (SRPK3) and the MIG repertoire of PM154 organoids included amongst others MAPK10, chromatin remodeling factors such as ACTL6B and several proteins involved in cytoskeletal signaling (CEP170, EML4, PDE6D) but also proteins involved in DNA damage repair (MSH3). One could thus argue that as PCa progresses, the cellular MIG pool favors cancer survival. Off note, the inventors also compare endogenous AS events in the RNAseq data of the samples treated with scrambled siRNA found only one AS event that is significantly different in all four cell lines, which is occurring more often in PM154. This event occurs in TSPYL2 which recently was shown to contribute to abiraterone, an ARSi, resistance in CRPC via CYP gene transcription regulation.

### Example 10: p38MAPK Regulates MiS in PCa-NE

To answer the question why CRPC-NE cells/organoids display high MiS activity although they usually have low AR activity and if there are multiple mechanisms to regulate MiS activity, the inventors explored an AR independent mechanism of MiS regulation in NEPC. pP38MAPK was recently found to trigger neuroendocrine differentiation in LNCaP cells. Moreover p38MAPK has been implicated in therapy-resistant PCa cells to enhance invasion, metastasis, and immune evasion. Interestingly, p38MAPK is in part regulated by the rapid turnover of U6atac snRNA. The Dreyfuss lab showed that anisomycin mediated activation of the p38MAPK results in increased U6atac stability thereby activating the MiS. Based on these lines of evidence, the inventors hypothesized that in NE organoids p38MAPK regulates MiS activity.

To address this, the inventors tested this effect of anisomycin² to activate p38MAPK in LNCaP and C4-2 PCa-adeno cells, and H660 PCa-NE cells as well as in 5 of the inventors' organoids (MSK8, MSK10, MSK16, PM154 and PM1262) transfected with a luc-splicing reporter assay constructs. According to a recently published study on patient derived organoids from MSKCC (designated MSK) and The inventorsill Cornell (designated PM), Tang et al. classify MSK8 as stem-cell like PCa-adeno, MSK16 and PM1262 organoids group into organoids which enrich for WNT signalling and MSK 10 and Pm154 genomically enrich for a NEPC signature. Surprisingly, the effect on minor intron splicing was only observed in PCa-NE cells and organoids (H660, PM154 and MSK10) thereby suggesting that MiS activity in neuroendocrine PCa might be regulated through the p38MAPK axis FIG. 22A and D.

To directly test this, the inventors explored the expression and minor intron splicing of all 4 p38MAPKs (MAPK11,12,13 and 14) in the inventors' omincs datasets. Integration of both siU6atac RNAseq and proteomics showed that MAPK14 and 13 might be the key components that could be leveraged to regulate MiS activity. The inventors found that all 4 p38MAPK family members display intron retention or AS in LNCaP, C4-2, 22Rv1 and PM154 cells and organoids upon a U6atac KD. Comparing RNAseq with proteome data, the inventors found that MAPK14 protein expression however is only decreased in 22Rv1 cells which are considered an intermediate between adeno and neuroendocrine stages and MAPK13, which promotes neurotoxicity and is required for prostate epithelial differentiation was only decreased in PM154 organoids.

Therefore, the inventors performed siRNA mediated KD of MAPK14 or 13 in LNCaP, C4-2, 22Rv1 and H660 cells as well as in MSK8,10,16, PM154 and PM1262 organoids along with the luc-splicing reporter assay (FIG. 22B). The inventors discovered that MAPK13 downregulation did not show significant minor splicing changes as reflected by luciferase activity of the minor intron reporter. siMAPK14 decreased minor splicing significantly only in H660 cells and PM154 organoids. Together these findings reveal a novel mechanism of regulating MiS in neuroendocrine PCa in the absence of AR. Importantly the major splicing reporter construct did not show significant changes. In summary, the inventors now show that MiS is regulated in a bimodal fashion where AR activity informs MiS in prostate adenocarcinoma. While MAPK13 and 14 is one potential regulator of MiS activity in neuroendocrine PCa (FIG. 22D).

### Example 11: Pathway for Minor Intron Splicing Related to Prostate Cancer Progression

To answer the question whether MIGs can influence mRNA levels within the transcriptome and whether U6atac is not impacting gene expression through its function in the MiS. Currently U6atac is only known to regulate minor intron splicing through its interaction with U4atac as part of the MiS. Knockdown of U6atac was predicted to result in elevated minor intron retention which was captured by the inventors' RNAseq analysis (FIG. 3).

It is surprising that in this case the inventors do not observe significant downregulation in expression levels of MIGs with intron retention. However, there are multiple explanations for this outcome. First major NMD players including UPF1 and NCBP2 are themselves MIGs that show aberrant splicing. Thus, NMD pathway itself is compromised thereby allowing these aberrantly spliced transcripts to escape NMD, which is reflected in the lack of downregulation of MIGs. Despite this lack of downregulation, the aberrant splicing of MIGs will affect the function of the encoded protein because the majority of the transcripts fail to encode the full-length protein. In fact, the inventors have previously shown that aberrant splicing of MIGs through MiS inhibition results in transcripts that escape mRNA surveillance mechanisms⁸. Moreover, the inventors have previously also shown that aberrantly spliced MIG transcripts are bound to the ribosome as such production of novel potentially toxic proteins might contribute to the observed cell cycle defects. Finally, there is new literature support for intron retention as a distinct form of regulation that is not just restricted to NMD.

To analyze MIGs influencing transcriptome, MIGs execute varies functions that are disrupted upon aberrant splicing of these MIGs. For example, MIGs are splicing factors (SFs), subunits of polymerase II and III (Pol II, III), transcription factors (TFs), RNA binding proteins (RBPs), chromatin remodeling factors (CRFs) and ribonucleoprotein particles (RNPs). In all, U6atac KD results in aberrant splicing of MIGs, which partly escape NMD and are not observed to be downregulated, end up impacting transcription directly and/or indirectly the transcriptome of the cell.

As described above siU6atac mediated aberrant splicing of MIGs has a global transcriptomic effect. Since the inventors' objective was to understand how the primary defect of inhibiting MiS activity manifests in the cancer cell to ultimately result in cell cycle defect and cell death, the inventors endeavored to capture the overall transcriptome changes that underpin the cell cycle defect and cell death. Thus, initially the inventors emphasized genes either MIGs or non-MIGs that are affected by siU6atac.

Next, the inventors added additional PCa relevant MIGs to the listing such as JNK2 and JNK3, E2F1 and E2F2, RNPC3, Brd9, BRAF, ACTL6A, SMARCA1 and ACTL6B (also known as BAF53b), which the inventors recently described as a crucial NEPC biomarker. Below is stated a table that underlines the dynamic and context dependency of minor splicing in different PCa cell lines (Table 1). In that sense the inventors found that siU6atac triggered context dependent decrease in expression of many MIGs and non-MIGs with known functions in NEPC transdifferentiation such as the previously mentioned p38MAPK or BAF53B and BAF53A, EED, EZH2, AURKA which is likely based on intron retention and AS events (Table 1).

| | LNCaP | C4-2 | 22Rv1 | PM154 |
|---|---|---|---|---|
| ACTL6A (MIG) | Transcript | Transcript | Only retention or | Only retention or |
| | downregulation | downregulation | AS | AS |
| ACTL6B (MIG) | — | — | Only retention or | Only retention or |
| | | | AS | AS |
| EED (MIG) | Only retention or | retention or AS, | retention or AS, | Only retention or |
| | AS | protein decrease, | protein decrease, | AS |
| | | transcript | transcript | |
| | | unchanged | unchanged | |
| EZH2 (non-MIG) | — | Transcript | Transcript | — |
| | | downregulation | downregulation | |
| AURKA (non- | Transcript | Transcript | Transcript | Protein decrease |
| MIG) | downregulation | downregulation | downregulation | |
| | | | and protein | |
| | | | decrease | |

### Example 12: Minor Splicing Relates to a Decrease in Cell Cycle Genes

The primary defect in cell cycle is mediated by MIGs that are actively participating in the regulation of cell cycle. Importantly the inventors show that MIGs with elevated retention that are also downregulated at the protein level highly enrich for cell cycle regulation (GO-Term enrichment analysis). Thus, the primary molecular defect driving the observed cell cycle defect is aberrant splicing of MIGs. Additionally, it is now well established in the literature that disruption of minor splicing is associated with cell cycle defects, affecting S-phase, mitosis, and cytokinesis during brain development. Given that the inventors have demonstrated that siU6atac results in minor intron splicing defects similar to that observed in other model systems it is not a stretch to connect aberrant splicing to the observed cell cycle defects in PCa. Indeed, this is reflected in the inventors' molecular transcriptome/proteome intersection analysis (FIG. 4). Together the inventors' data in PCa thus nicely lines up with the literature connecting aberrant splicing of MIGs to cell cycle in developing cortex and limb.

### Example 13: Differential Clustering of Cancer Types

Next, the inventors found that differential clustering noticeably improves when including more cancer types (and thus samples). In particular, the disparities in clustering based on MIGs and non-MIGs grew as a result of including more cancer types (i.e., the differences in Silhouette scores between MIGs and non-MIGs grew after including more cancer types in the inventors' analysis). The results from this revised analysis are given in FIG. 25.

FIG. 4B suggests that there may exist a strictly linear relationship between the fraction of MIGs within a sample and the resulting Silhouette scores (in contrast to potentially more complex, non-linear relationships such as those that may suggest saturation, for example). In order to evaluate the extent to which the fraction of MIGs indeed influence Silhouette scores in a linear fashion, the inventors used simple linear regression to regress the median Silhouette scores on the fraction of MIGs. Specifically, the inventors simply take a given median Silhouette score (for a specific fraction of MIGs) to be the median score among those associated with the same 1000 resampled gene sets that had been used to generate FIG. 4B. The results from this regression strongly a linear relationship, and the inventors provide the associated summary statistics below:

t-statistic and p-value associated with the slope:
t-stat: 64.97
p-val: 2.45e-13
R_squared: 0.9979
F statistic: 4221 (on 1 and 9 degrees of freedom)

Residual standard error: 0.0005889 on 9 degrees of freedom

Specifically, the inventors chose to include only those cancer types for which a sufficient number of samples with expression data are available. The inventors now only enforce that each cancer type be represented by a sufficiently large number of samples (we chose a threshold of 18). This enabled the inventors to retain 23 cancer types across a sample size of N=1224.

### Example 14: MiS Influences REST Splicing

To answer the central question in the field of understanding PCa drug resistance to ARSi, whether MiS is indeed linked to the CRPC/NE transition in Pca, the authors previously reported that 10-15% of CRPC-adenos undergo lineage plasticity to a AR negative state, some express neuroendcrine surface markers and others are double negative. For neuroendocrine transdifferentiation, REST is a known critical factor.

RE1-silencing factor (REST) is a well-defined repressor of neural differentiation. Loss of REST expression has been associated with up-regulation of genes that are used to define CRPC-NE (e.g., SYP and CHGA). Moreover, loss of REST expression precedes neuroendocrine differentiation in PCa. As such loss of REST has been considered a potential NEPC driver PCa. Relevant to this study, REST is regulated through dynamic alternative splicing such that a miniexon in intron 3 when included results in a premature stop codon truncating the open reading frame. This truncated form of REST protein, referred to as REST4, cannot bind to the RE1 silencing element but can block REST FL/DNA contact. REST4 thus acts as a dominant negative and consequently increased levels of REST4 would result in inhibition of REST function enabling NE differentiation. The inventors observed that REST expression by qPCR is dynamic across the four different cell lines representing PCa progression (LNCaP>C4-2>22Rv1>PM154) (FIG. 25A). Importantly when the inventors compared REST4 expression, the upregulation of this isoform in 22Rv1 and PM154 was highly significant (FIG. 25A). Analysis of the SU2C data matches these results (FIG. 25B). The inventors found that canonical REST positively correlates with the AR score while it displays a negative correlation with the NEPC Score. In line with this REST expression was significantly decreased in NEPC-samples (NEPC score >0.4) when compared to AR-samples (AR-score >0.4). In contrast, when the inventors queried SU2C for REST isoform expression the inventors found that REST4 negatively correlates with AR- and positively correlates with NEPC-Score. Here REST4 expression was significantly increased in NEPC-samples (NEPC score >0.4) when compared to AR-samples (AR-score >0.4).

When the inventors related this information to minor splicing, the inventors found that U6atac KD increases canonical REST after 96 h and decreases REST4 expression after 48-72 h in NE-like cells (22Rv1) and the NE-organoids (PM154) (FIG. 25C and D). Surprisingly, there was no change at the overall gene expression of REST in the inventors' RNAseq data of scrambled and siU6atac cells. However, the inventors did observe compensatory changes in two alternatively spliced REST isoforms after siU6atac (48 h) treatment (REST4 and REST′3c+) (FIG. 25D-F); MiS inhibition through siU6atac resulted in downregulation of REST4 in 22Rv1 and PM154 cells and organoids (FIG. 25D-F). As described above REST4 upregulation acts as a dominant negative to block REST function. This finding suggests that MiS not only correlates with PCa progression but might be involved in the CRPC to NEPC transition via regulation of alternative REST splicing. Thus the inventors are able to now show that MiS is indeed involved in CRPC/NE transition via regulation of alternative REST splicing.

### Example 15: AR and Minor Splicing

First, the inventors show that MIGs are crucial players in executing the oncogenic program downstream of PCa causing genes. This placement of MiGs positions the MiS as an important target for PCa therapeutics. Specifically, the inventors show that not only is the MiS directly downstream of AR-axis which blocks U6atac turnover (FIGS. 2 and 26A), but inhibition of MiS results in downregulation of AR protein (FIGS. 4, 26B). Taken together this finding shows the intimate relationship of the MiS in AR driven PCa progression. Therefore, the inventors suggest that MiS is a therapeutic target for CRPC-adeno. Moreover, it is known that REST function is critical for progression to AR independent CRPC-NE. In this regard, please see the previous paragraph. Once CRPC-NE is established p38MAPK has been shown to play a critical role in MiS regulation (FIG. 21). To sum up the inventors think that the MiS is regulated by different oncogenes (AR, p38MAPK, etc.) and intertwined with tumorigenic pathways. Thus, the therapeutic window in context of PCa is multifaceted and can be leveraged to treat both CRPC-adeno and CRPC-NE.

### Example 16: MiS Inhibition Affects PCa Cells Over Normal Cells

Next, the inventors revisited the cell doubling (FIG. 27) and performed Incucyte experiments with all cell lines that were used FIG. 3. Briefly confluence of cells treated for 120 h with siU6atac or siScrambled was measured every 6 h. At the end of each Incucyte experiment viability of every sample was tested by cell titer glow (CTG) assays. FIG. 26 shows Incucyte and CTG results (y-axis) as x-fold of the scrambled control plotted against the doubling time of the cell lines (x-axis). Cell lines were ranked according to their doubling time. The inventors did not observe a correlation between cell growth (doubling time) and siU6atac response. Thus, the inventors conclude that while minor splicing definitely impacts cell cycle and proliferation, MiS dependency does not merely track with the proliferative rate of a cell. Instead, MiS dependency is cancer type and stage specific. Indeed the inventors also show that PCa specific oncogenic drivers such as AR, REST, P38MAPK are all intertwined with MiS activity and might play a role in defining miS dependency. Taken together, the inventors conclude that MiS inhibition will disproportionally affect PCa cells over normal cells.

### Example 17: siU6atac Mediated MiS Inhibition is Affected in PCa

In agreement with the inventors' assumption that MiS inhibition will disproportionally affect PCa cells over normal cells the inventors show that MS2514 (normal mouse prostate cells), that have a low doubling time (FIG. 26) and HS27 (normal human fibroblasts), which have a high doubling time (FIG. 26) do not show a significant response to siU6atac (FIG. 26 and FIG. 3A). Importantly, human fibroblasts HS27, have a doubling time similar to LNCaP or 22Rv1 cells, yet are significantly less sensitive towards a U6atac KD.

To further address tumor selectivity, the inventors performed co-culture experiments with C4-2-GFP cancer cells and HS27-mCherry fibroblasts (FIG. 27). Briefly C4-2 cells were fluorescently tagged with green fluorescent AIEDot nanoparticles while HS27 cells were tagged with red fluorescent AIEDot nanoparticles. Subsequently both cell lines were co-cultured and then treated with Scrambled or U6atac siRNA. Cell growth was measured every 6 h for a total of 5 days. At the end of the experiment the cell mix was FACS sorted to separate C4-2 and HS27 cells and U6atac KD was confirmed via FACS sorting and subsequent qRT-PCR. As expected siU6atac decreased green fluorescent signal (C4-2) significantly but did not impact the red fluorescent signal, even though U6atac KD was stronger in HS27 as compared to C4-2 cells (FIG. 27).

Taken together the inventors show that siU6atac mediated MiS inhibition is not just linked to proliferative rate, rather it is specifically affected in PCa as it uses a lot of MIGs to execute the oncogenic program. In that sense the inventors are currently building infrastructure to address the issue of therapeutic window to titrate MiS inhibition for translating the inventors' discovery into the clinics.

### Example 18: Molecular Splicing Defects of Inhibiting the MiS

In PCa the inventors found that MiS downstream signaling encompasses critical MIGs such as BRAF; PARP1, EED, ACTL6A and B, MAPK8, 9, 13 and 14. The exact MIG repertoire is however cell type dependent. In that sense the inventors found for example that a U6atac KD triggers a decrease of ACTL6A transcript in LNCaP and C4-2 cells but not in 22Rv1 and PM154 cells (FIGS. 3 and 4). The paralogue of ACTL6A, ACTL6B however displayed increased intron retention after siU6atac treatment in 22Rv1 and PM154 cells but not in LNCaP and C4-2 cells. Similar siU6atac triggered an increase of MAPK9 in hormone responsive LNCaP cells but a decrease in therapy resistant C4-2, 22Rv1 and PM154 cells (FIG. 4 A-E) whereas PARP1 displayed intron retention in all 4 cell lines.

Here the inventors identified PCa relevant MIGs via RNAseq, qRT-PCR (FIG. 3), masspec and immunoblot analysis in U6atac KD cells FIG. 4, FIG. 26 (PARP). In all the inventors rely on the disruption of a repertoire of MIGs that the inventors show is a novel therapeutic strategy for PCa.

In order to show that disruption of the PCa relevant MIGs primarily goes through U6atac the inventors have performed rescue experiments (FIG. 8A). The inventors created C4-2 cells that stably expressed a doxycycline (dox) inducible hairpin against U6atac. The inventors dox-treated the cells as well as the shScrambled control cells for 48, 72 and 96 hours with 2 ug/ml doxycycline, froze half of the cells as U6atac KD, washed the other half 3 times with PBS to get rid of doxycycline and re-cultured for another 72 hours (U6atac KD rescue cells). Finally, the inventors performed qPCR against U6atac as well as several MIGs (CoA3, PARP, EED, ACTL6B, PTEN). In line with the inventors' hypothesis that siU6atac decreases MIGs critical for PCa depleting doxycycline from the media rescued the shU6atac triggered decrease in U6atac and in the tested MIGs. However, the inventors only observed a decrease after 48 h. At later timepoints the inventors observed an gradual increase of all genes tested which might be due to a potential positive feedback here. Unlike the siRNA the inventors noticed that shU6atac, which reproduced elevated minor intron retention, did not result in significant cell death. Indeed the differences in efficacy of shRNA and siRNA are inherent due to the underlying mechanism through which they act. However, this experimentation allowed the inventors to show the molecular splicing defects of inhibiting the MiS.

### Example 19: U6atac KD Causes Observed Cancer Death

To test whether the inventors can rescue the cell death phenotype observed in siU6atac experiments, the inventors used CRISPR mediated KO of endogenous RNU6ATAC in cells overexpressing U6atac snRNA from a transfected plasmid in C4-2 cells (C4-2′U6atac cells) (FIG. 9A and B). The inventors designed 2 gRNAs targeting sites flanking the RNU&6ATAC gene (FIG. 9). The rational here is that only endogenous U6atac will decrease while cells that express the U6atac Plasmid will still express functional U6atac (FIG. 24).

As expected overexpression of U6atac plasmid increased growth of C4-2 cells significantly (FIG. 9C). In line with that assumption C4-2′U6atac cells did not show a decrease in U6atac expression even though the gene was CRISPERed out successfully (FIG. 9E and F). In line with the inventors' siRNA based U6atac KD approach a KO of U6atac triggered cell death in C4-2 cells but not in C4-2′U6atac cells (FIG. 9G and H) which verifies that U6atac KD is the cause of the inventors' observed cancer death.

### Example 20: Pulldown Experiments with Anti-PDCD7 ABs

The inventors performed the pulldown experiments with anti-PDCD7 ABs for 2 reasons: 1) anti-PDCD7 so far is the only reliable AB among all 7 unique MiS protein ABs. 2) Unlike major intron 5′ splice sites, minor intron 5′ splice sites are initially recognized and bound by a protein which is PDCD7. Since the inventors wanted to pull-down the pre-mRNAs, the inventors chose to perform the PD with this direct interactor protein.

For 1H and 11 the inventors have run the correlation analysis for all 7 proteins unique to the MiS as well as for ZRSR2 and CENPA which are proteins shared by minor and major spliceosome (FIG. 30). So far KD of ZRSR2 and CENPA studies have reported the predominant defect is actually in minor intron splicing.

Of course, ZRSR protein loss of function in the testis has also reported major intron defect. Intriguingly the inventors observe while the majority of MiS proteins shows a trend towards a positive correlation with EZH2, ZMAT5 and PDCD7 show a trend towards a negative correlation with EZH2. Together these findings show that MiS and its components are dynamically regulated across PCa progression and the combinatorial changes in expression of the MiS components need further exploration. Moreover, these proteins have not been extensively studied as such the inventors do not understand other potential functions they might execute. For example, PDCD7, as the name suggests was identified as Programmed Cell Death Protein 7 gene and was reported as a potential transcription factor that activates the transcription e-cadherins. In all this analysis is one of the first to begin to place the entire MiS components in PCa progression. One caveat of course is that proteins of the MiS can have multiple functions, but snRNAs are currently thought to be exclusively involved in executing minor splicing.

### Example 21: Number of MIGs Expressed in Cell Lines

Here the inventors identified the number of MIGs expressed in all three cell lines (LCR-515 MIGs) or the PM154 organoid (555 MIGs) and submitted these lists to DAVID, which yielded 81 and 99 significant GO terms, respectively. The inventors then intersected these GO terms, which reflect the functional enrichment of MIGs at baseline, with those generated by MIGs with elevated minor intron retention in the cell lines (39 GO terms) and the organoid (42 GO terms). For the cell lines, this intersectional approach identified 54 GO terms unique to MIGs expressed at baseline, 12 were unique to MIGs with retention, and 27 were shared. For the organoid, 70 GO terms were unique to MIGs expressed at baseline, 13 were unique to MIGs with retention, and 29 were shared. For both the cell lines and the organoid, the inventors found that: GO terms unique to MIGs expressed at baseline notably included cell cycle; spliceosomal complex assembly, RNA metabolic processes, DNA repair, small GTPase binding etc., shared GO terms included MAP kinase activity, nucleotide excision repair, snRNA transcription from RNA Polymerase II promoter, ATP binding, nuclear pore etc.; and GO terms unique to MIGs with retention included RAN GTPase binding, protein serin/threonine kinase activity, polA RNA binding and protein transport. Thus, the enrichment of pathways by mis-spliced MIGs reflects only some of the pathways that MIGs regulate at baseline, and also includes novel terms that do not emerge when only focusing on MIGs expressed at baseline.

### Example 22: Conclusion

In the PCa field there is active debate on classification on PCa stages. Therefore, the inventors have presented the classification scheme of the different PCa cell lines based on Bluemn et al. to clarify the inventors' analysis (FIG. 29). 22Rv1 represent an intermediate PCa state, they are AR positive but do express several NEPC markers. In other words, they bear some degree of transcriptomic similarities to neuroendocrine PCa cell lines (FIG. 31) thus the inventors placed them at the transition from CRPC to NEPC in the graph. C4-2 and 22Rv1 labels were exchanged in FIG. 2B to be consistent with previous ordering.

Here the inventors report for the first time that siU6atac and MiS inhibition is a potential therapeutic target. It will take considerably effort and time to translate this finding into the clinic to specifically treat therapy resistant lethal PCa. The inventors wanted to therefore show that this novel strategy is indeed a therapeutical viable approach by comparing it to the most exciting possible therapeutics in the field such as the combination of enzalutamide with EZH2 inhibition. This combinational approach is not FDA/EDA approved but there are several Phase 1 and 2 trials exploring this combination in CRPC. This combination approach is based on the demonstration by several groups (Mu, P. et al., Science 2017, 355 (6320), 84-88; Ku, S. Y et al., ibid) that EZH2 inhibitors re-sensitize therapy-resistant PCa towards enzalutamide treatment. Therefore, the significance of the finding of FIG. 6G is to underscore that siU6atac and MiS inhibition holds high therapeutic value and potential. The inventors have clarified this in the text.

### Example 23: Discussion

MIG-expression discriminates cancer from benign tissue. MIG-expression and the pathways they regulate are intimately linked to oncogenes (FIG. 1A). Thus, MIGs are critical bottlenecks for disparate molecular pathways downstream of these oncogenes, and as such are part of the aberrant regulatory logic of cancer related phenotypes. Therefore, the MiS likely represents another master regulator of cancer whose inhibition would trigger tumour checkpoint collapse. Indeed, MIG expression was discriminatory between different cancer types, while levels of minor intron retention were informative in PCa progression (FIG. 1). Thus MIGs, with diverse functions, are ideal effectors of the disparate pathways driven by cancer-causing genes. Moreover, minor intron splicing is potentially a crucial regulatory node for cancer progression, which is consistent with the idea of increased cell cycle speed that cancer progression necessitates. In agreement with these findings, the inventors found MiS snRNAs (crucial regulators of MiS activity) to be upregulated in cancer and across cancer progression (FIG. 1 and FIG. 2A). These findings suggest that MiS activity is enhanced and actively regulated across cancer progression. Moreover, increased U11, PDCD7 and RNPC3 expression have been associated with PCa, AML and increased anti-RNPC3 cancer associated scleroderma respectively. Even more drastic upregulation was observed for the MiS catalytic core snRNA U6atac, which is controlled through turnover rather than transcriptional upregulation. According to the “molecular switch theory,” U6atac is rapidly turned over and, as such, is the limiting snRNA that dictates MiS activity. Cellular stress signalling and, among others, p38MAPK activation, causes a rapid increase in U6atac expression along with enhanced splicing of MIGs that are otherwise inefficiently spliced or degraded. Thus, stabilization of U6atac, which elevates its levels, can enhance minor spliceosome activity. Re-phrased, U6atac levels can control MIG expression “on-demand” when required. Consistent with this model, U6atac upregulation was observed in tumours from various tissues compared to their respective benign counterparts (FIG. 1). This finding underscores the idea that U6atac snRNA levels are a crucial regulator of minor spliceosome activity during cancer progression.

MiS activity increases with cancer progression. MIG expression is uniquely dependent on the splicing efficiency of minor introns. As such, it is not surprising that the inventors found differential efficiencies for the splicing of minor introns when the inventors compared different PCa subtypes. Indeed, studying U6atac levels during progression of prostate cancer showed that U6atac expression is closely correlated to the progression of Pca and is positively associated with Pca metastasis (FIG. 2A). Support for this idea was observed in minor intron splicing reporters, which were more efficiently spliced in cell lines representing more aggressive cancer stages (FIG. 2B). For example, splicing of minor introns, unlike major introns, was more efficient in different therapy-resistant CRPC-adeno cells and in various CRPC-NE organoids (FIG. 2B). Considering the “molecular switch theory,” MIG regulation may thus represent a dynamic mechanism of adaptation by cancer cells in response to changing environmental conditions, as seen in therapy resistance. In this sense, the inventors found that MIG splicing increases with prolonged ARSi treatment of prostate cancer. Resistance to ARSi is almost always based on a re-activation of the AR axis in prostate cancer, which lines up with the inventors' data showing that MIG splicing is regulated by the AR in PCa (FIG. 2D-F). These findings, along with the inventors' previous discovery of U6atac as a powerful modulator of MiS activity, strongly suggest that controlling U6atac levels is a point of therapeutic intervention. Together these findings reveal a unique role for MiS activity in the onset and progression of cancer and positions the MiS as a potential therapeutic target.

siU6atac blocks cell cycle of cancer cells. Given that U6atac snRNA has no other known function outside of the MiS, siU6atac specifically inhibited MiS function, which was reflected in elevated minor intron retention (FIG. 3). In fact, siU6atac resulted in the largest number of MIGs with elevated minor intron retention compared to published reports of MiS inhibition with other components. Unlike MiS inhibition through other components, the largest AS events observed in siU6atac samples were cryptic splicing events executed by the major spliceosome (FIG. 3D). MiS inhibition through siU6atac is expected to disrupt the U4atac-U6atac-U5 tri-snRNP that is recruited after 5′ splice site recognition by the U11/U12 di-snRNP. Thus, the exon definition requirement is mostly fulfilled and exon-skipping by the major spliceosome is less likely (FIG. 3D, red). This finding bolsters the idea that inhibition of the minor spliceosome induces specific types of AS events depending on the specific MiS component that is targeted and the cell type in which it is targeted. This finding opens a new way to target snRNAs, which allows inhibition of the minor spliceosome directly.

The cell type-specific response to MiS inhibition was reflected in the different numbers of MIGs with altered minor intron splicing for each cell line (FIG. 3C). Indeed, principal component analysis with either minor intron retention or AS showed that the response to MiS inhibition in C4-2 is closer to LNCaP, whereas 22Rv1 is closer to PM154 (FIG. 11). This finding is consistent with the inventors' understanding that LNCaP, C4-2, 22Rv1 and PM154 can be placed in that order in PCa progression. The separation of PM154 organoids from the three cell lines was also evident in the overlap analysis of minor intron retention and AS events (FIG. 3). STRING network analysis showed how simultaneous aberrant splicing of MIGs converge upon a central node composed of downregulated genes that predominantly enrich for cell cycle regulation. (FIG. 3). Interestingly, the same analysis performed for the PM154 organoids did not yield similar results. This suggests that MiS inhibition is indeed cell type specific, tumour stage and context dependent, which is consistent with hierarchical clustering of 9 cancer types using MIG expression (FIG. 1D). However, IPA analysis with PCa interacting MIGs and downregulated genes in the three cell lines and organoid showed distinct cancer-related biological pathways (FIG. 3). This underscores the idea that MiS inhibition can perturb disparate MIGs in different cell lines yet converge on the same biologically relevant endpoint for cancer (i.e., cell cycle and survival). For example, biological network analysis revealed a MAPK-centric network for the adeno cell lines whereas in the organoids a PARP1, ACTL6A, SRPK1 driven network was observed. The latter finding is consistent with the idea that the CRPC-NE organoids are molecularly distinct from the three CRPC-adeno cell lines.

Perturbation of predicted biological pathways based on transcriptomic changes are ultimately implemented by the proteins produced. Mass spectrophotometry analysis showed downregulation of many MIGs that were aberrantly spliced. However, the inventors did not observe a one-to-one correlation (FIG. 4), which is consistent with published reports about the discordance between the transcriptome and proteome. Nonetheless, proteins (MIGs and non-MIGs) that are up or downregulated in the siU6atac condition were relevant to PCa progression. For example, AURKA, which was shown to be important in CRPC differentiation and aggressiveness, was increased in therapy-sensitive LNCaP cells but decreased in CRPC-adeno cells 22Rv1 and CRPC-NE organoids PM154. Similarly, the polycomb group protein EED, known to regulate AR expression levels and a potential target in CRPC was decreased in C4-2 and 22Rv1 cells. In contrast, the inventors observed an increase in the protein levels for the G1 cell cycle arrest mediator CDN1A in all CRPC-adeno cells (FIG. 4A-E). While there is inherent discrepancy between the transcriptome and proteome, the inventors found that genes with downregulation of both transcript and protein highly enriched for cell cycle regulation and DDR GOTerms. Thus, despite the dynamic fluctuations in the levels of mRNA and protein, the core molecular defect is that of inhibition of proliferation. Indeed, FACS analysis confirmed G1/GO arrest in C4-2 cells and PM154 organoid (FIG. 4).

ScRNAseq reveals reduction in AR- and EMT-score in siU6atac-treated cells and organoids. Cell cycle defects observed by FACS analysis (FIG. 4) were further confirmed by scRNAseq analysis, which revealed a high number of siU6atac treated cells in G1-phase (FIG. 5). The inventors also observed increase in the number of siU6atac treated cells in S-phase, which is consistent with the enrichment of the DDR pathway. Again, these findings underscore the cell type specific effects of MiS inhibition that ultimately result in a block in proliferation, a key feature for any therapeutic strategy. Consistent with this, the inventors observed a decrease in EMT activity after MiS inhibition, which implies that targeting the MiS not only blocks cancer progression but also metastasis (FIG. 5 and FIG. 13). The main strategy in PCa therapy however is to block AR activity. Intriguingly, the inventors found that MiS inhibition led to reduced AR activity score in PCa cells and organoids (FIG. 5 and FIG. 14) This reveals inhibition of AR transcriptional activity as a novel mechanism of action of the MiS, which is potentially cancer type or even cancer stage dependent. Considering that MiS activity, and thus MiS-dependent signaling, is tissue and cancer type-specific, this finding indicates that the MiS may represent a dynamic mechanism of adaption for cancer cells in response to changing environmental conditions. The MiS may thus be a common denominator of prominent cancer driver axis such as the AR axis in PCa, that could be exploited as an all-in-one target for many cancer types.

The minor spliceosome is essential for PCa growth and viability. Whereas siU6atac-mediated MiS inhibition substantially impacted cancer cells, it did not strongly affect human fibroblasts or benign mouse prostate cells (FIG. 6A and B). This finding has therapeutic relevance as it appears that MiS inhibition is specifically detrimental to highly proliferative cells. Although U6atac KD resulted in less viability of LNCaP cells, their proliferative index was not significantly affected by a MiS inhibition (FIG. 6C). This difference in response by LNCaP cells can be explained by the slow rate of cell division, which has been shown to be dependent on AR activation. Consistent with previous results that nominated the AR as a regulator of MiS activity, the inventors noticed that AR overexpression increased proliferation of LNCaP cells, thereby making them more susceptible to siU6atac-mediated MiS inhibition (FIG. 6C). Thus, U6atac is a promising target for CRPC-adeno PCa with persistent (re)-activation of the AR⁵⁵. The inventors' study underscores the importance of inhibiting the MiS, which consists of other crucial components besides U6atac. Indeed, the inventors found that the use of siRNA against the unique MiS protein RNPC3 led to similar effects as seen with siU6atac in PCa (FIG. 6A). However, the inventors found that MiS inhibition can have differential effects based on both the cell type it is inhibited in and the MiS component targeted for its inhibition. For example, the inventors found that siU6atac, while being quite broadly effective, is best in PCa and glioblastoma cells (FIG. 6D). Similarly targeting other MiS components such as RNPC3 and PDCD7 showed significant results in PCa and glioblastoma cells (FIG. 18). In contrast, U12 KD had no significant effect on viability in all cell lines tested (FIG. 18).

Interestingly, U6atac KD alone did not impact bladder cancer cells, but it was very effective when combined with cisplatin (FIG. 6E and F). This finding suggests U6atac inhibition might be a valid addition for combination therapy for bladder cancer. Considering that many invasive bladder cancer patients are cisplatin-ineligible, a combination of cisplatin and siU6atac might represent a promising alternative to treat those patients. Similarly, enzalutamide-resistant CRPC could benefit from combination therapy that includes siU6atac. Enzalutamide alone increased CRPC growth in resistant cells, which emphasizes the need for new CRPC treatments (FIG. 6G). For instance, it has been shown that EZH2 inhibition overcomes enzalutamide resistance in cultured PCa cells and xenograft models. Surprisingly, the inventors found that U6atac KD by itself is sufficient to block proliferation of CRPC cells, which is better than enzalutamide, siEZH2 or siEZH2/enzalutamide (FIG. 6G). Importantly, the inventors show that MiS inhibition also works in PCa patient-derived organoids (CRPC-adeno and CRPC-NE), whose transcriptome signature correlates with those from patients tested in the SU2C study (FIG. 6H).

Taken together, the inventors show that MiS activity plays a crucial role in the progression of PCa and that MiS inhibition is a viable therapeutic target. The inventors show that inhibiting different MiS components can block cancer cell proliferation and viability, but in the inventors' study, U6atac was the most effective. The inventors show that MiS activity corresponds to AR signalling across stages of PCa progression, which is reflected by the increase in U6atac expression. Indeed, this discovery suggests that U6atac could also be employed as a diagnostic marker for lethal PCa and other cancers. Regardless of the stage of PCa, siU6atac can successfully inhibit proliferation and viability through disruption of pathways such as MAPK, cell cycle and DNA repair. While in the current study PCa is used as a model to investigate the efficacy of MiS inhibition as a therapeutic strategy, these observations could extend to other cancer types.

### Example 24: Material and Methods

### Cell and Organoid Lines

LNCaP (male, ATCC, RRID: CVCL_1379), C4-2 (male, ATCC, RRID: CRL-3314), 22Rv1 (male, ATCC, RRID: CRL-2505), PC3 (male, ATCC, RRID: CRL-3470), DLD-1 (2 (male, ATCC, RRID: CCL-221), L-rENZ and L-AR cells were maintained in RPMI medium (Gibco, A1049101), supplemented with 10% FBS (Gibco, 10270106), and 1% penicillin-streptomycin (Gibco, 11548876) on poly-L-lysine coated plates. RWPE cells (male, ATCC, RRID: CVCL_3791) were maintained in Keratinocyte Serum Free Medium (Gibco, 17005075) supplemented with bovine pituitary extract and human recombinant EGF (included), and 1% penicillin-streptomycin (Gibco, 11548876). HEK293T cells (female, ATCC, RRID: CVCL_0063), VCaP (male, ATCC, RRID: CRL-2876), MDA-MB-231 (female, ATCC, RRID: HTB-26), K-562 (female, ATCC, RRID: CCL-243), LN-18 (male, ATCC, RRID: CRL-2610) PC-3M-Pro4 and DU145 cells (male, ATCC, RRID: CVCL_0105) were maintained in DMEM (Gibco, 31966021), supplemented with 10% FBS, and 1% penicillin-streptomycin. NCI-H660 cells (male, ATCC, RRID: CRL-5813) were maintained in RPMI medium (Gibco, A1049101), supplemented with 5% FBS, 1% penicillin-streptomycin (Gibco, 11548876), 0.005 mg/ml Insulin (Sigma-Aldrich, 19278), 0.01 mg/ml Apo-Transferrin (Sigma-Aldrich, T1147), 30 nM Sodium selenite (Sigma-Aldrich, S9133), 10 nM Hydrocortisone (Sigma-Aldrich, H6909) 10 nM beta-estradiol (Sigma-Aldrich, E2257) and L-glutamine (for final conc. of 4 mM) (Sigma-Aldrich, G7513). PC-3M-Pro4 cells were a kind gift from Dr. Kruithof-De Julio. LNCaP-AR cells were a kind gift from Dr. Sawyers and Dr. Mu (Memorial Sloan Kettering Cancer Center). L-ENZ cells were established through constant enzalutamide exposure. Briefly low passaged LNCaP cells were treated over night with 20 uM enzalutamide in C/S media. The media was exchanged to normal RPMI (10% FBRS, 1% P/S) the next day and surviving LNCaP cells (˜10%) were maintained until they reached a confluency of ˜80%. This procedure was repeated twice. Subsequently the enzalutamide concentration was increased for three treatments to 40 uM and for 25 treatments to 80 uM. Cells are treated since them once a week with 80 uM enzalutamide.

All cell lines were grown at 37° C. with 5% CO₂. All cell lines were authenticated by STR analysis and regularly (every 3 month) tested for mycoplasma.

MSKCC-PCa8,10,14 and 16 CRPC-Adeno patient derived organoids were a kind gift from Dr. Chen (Memorial Sloan Kettering Cancer Center). All organoids including WCM154 were maintained in three-dimension according to the previously described protocol (Puca, L. et al., Nat Commun 2018, 9 (1), 2404; Gao, D., et al., Cell 2014, 159 (1), 176-187). Briefly Advanced DMEM (Thermo Fisher Scientific, 31966047) with GlutaMAX 1× (Thermo Fisher Scientific, 35050061), HEPES 1 mM (Thermo Fisher Scientific, 15630056), AA 1× (Life Technologies, 15240-062), 1% penicillin-streptomycin, B27 (Thermo Fisher Scientific, 17504001), N-Acetylcysteine 1.25 mM (Sigma-Aldrich, A9165), Recombinant Murine EGF 50 ng/ml (PeproTech, 315-09), Human Recombinant FGF-10 20 ng/ml (Peprotech, 100-26), Recombinant Human FGF-basic 1 ng/ml (Peprotech, 100-18B), A-83-01 500 nM (Tocris, 29-391-0), SB202190 10 μM (Sigma-Aldrich, S7076), Nicotinaminde 10 mM (Sigma-Aldrich, N0636), (DiHydro) Testosterone 1 nM (Fluka, 10300), PGE2 1 μM (Tocris, 2296), Noggin conditioned media (5%) (PeproTech, 120-10C) and R-spondin conditioned media (5%) (PeproTech, 315-32). The final resuspended pellet was mixed with growth factor-reduced Matrigel (VWR, BDAA356239) in a 1:2 volume ratio. Droplets of 40 pl cell suspension/Matrigel mixture were pipetted onto each well of a six-well cell suspension culture plate (Huberlab, 7.657185) To solidify the droplets the plate was placed into a cell culture incubator at 37° C. and 5% CO₂ for 30 min. Subsequently 3 ml of human organoid culture media was added to each well. 50% of the media was exchanged every 3-4 day during organoid growth. organoids were passaged as soon as they reached a size from 200 to 500 um. To this end, organoid droplets were mixed with TrypLE Express (Gibco) and placed in a water bath at 37° C. for a maximum of 5 min. The resulting cell clusters and single cells were washed and re-cultured, according to the protocol listed above.

### In Situ Validation Collection

Tissue micro-arrays were kindly provided by the Translational Research Unit (TRU) Platform, Bern (www.ngtma.com). For PCa the inventors used TMAs from the Bern PCBM cohort (Briganti, A. et al., Eur Urol 2013, 63 (4), 693-701) (28 patients) and a tissue microarray of 210 primary prostate tissues, part of the European Multicenter High Risk Prostate Cancer Clinical and Translational research group (EMPaCT) (Tosco, L. et al., Eur Urol Focus 2018, 4 (3), 369-375; Chys, B. et al., Front Oncol 2020, 10, 246; Dawson, H. et al., Histopathology 2020, 76 (4), 572-580).

### Mass Spectrometry Analysis

LNCaP, C4-2, 22Rv1 and PM154 cells (400 000) were seeded in a 6 well and treated for 96 hours with siScrambled or siU6atac RNA (16 pmol). 96 hours post transfection cells were harvested and 50% of the cell pellet was used for U6atac KD confirmation by qRT-PCR. The remaining pellet was washed twice with PBS and subjected to mass spectrometry (MS) analysis:

Cells were lysed in 8M urea/100 mM Tris pH8/protease inhibitors with sonication for 1 minute on ice with 10 seconds intervals. The supernatant was reduced, alkylated and precipitated overnight. The pellet was re-suspended in 8M urea/50 mM Tris pH8 and protein concentration was determinate with Qubit Protein Assay (Invitrogen). 10 μg protein was digested with LysC 2 hours at 37C followed by Trypsin at room temperature overnight. 800 ng of digests were loaded in random order onto a pre-column (C18 PepMap 100, 5 μm, 100A, 300 μm i.d.×5 mm length) at a flow rate of 50 μL/min with solvent C (0.05% TFA in water/acetonitrile 98:2).

After loading, peptides were eluted in back flush mode onto a home packed analytical Nano-column (Reprosil Pur C18-AQ, 1.9 μm, 120A, 0.075 mm i.d.×500 mm length) using an acetonitrile gradient of 5% to 40% solvent B (0.1% Formic Acid in water/acetonitrile 4, 9:95) in 180 min at a flow rate of 250 nL/min. The column effluent was directly coupled to a Fusion LUMOS mass spectrometer (Thermo Fischer, Bremen; Germany) via a nano-spray ESI source.

Data acquisition was made in data dependent mode with precursor ion scans recorded in the orbitrap with resolution of 120′000 (at m/z=250) parallel to top speed fragment spectra of the most intense precursor ions in the Linear trap for a cycle time of 3 seconds maximum.

### Generation of U6atac Overexpressing Cell Lines

LV290591-RNU6ATAC Lentiviral Vector (Human) (CMV) (pLenti-GIII-CMV-GFP-2A-Puro) as well as the corresponding empty vector control were purchased from ABM. DNA was amplified via chemical transformation of One Shot Machi T1 Phage-Resistant Chemically Competent E. coli cells (Invitrogen, C862003). Lentivirus was produced in HEK293T cells by transfection with the constructs, and subsequent virus containing media was used to transduce C4-2 cells. Three days post transduction the cells were subjected to puromycin selection (1 pg/mL). After the selected cells reached a confluence of 80%, they were FACS sorted for GFP positivity. This was repeated 3 times.

### Drug Treatments

For DHT stimulation experiments cells were starved of hormone for 48 hours in phenol red-free RPMI media (Gibco, 11-835-030) with 10% charcoal stripped FBS (Gibco, A3382101), then treated with 10 nM dihydrotestosterone (Fluka, 10300) for 24 hours.

For long-term ADT treatment cells were exposed weekly to 20 μM enzalutamide (Selleck Chemicals, S1250) or 10 uM Abiraterone.

For growth experiments cells were treated with siRNA and two hours later with 20 uM enzalutamide. Enzalutamide was refreshed 3 days later.

For splicing reporter assays cells were exposed to Anisomycin 1 ug/ml for 4 h (Sigma Aldrich, A9789).

### Proximity Ligation Assay

L-AR cells (50 000) were seeded in p-Slide 8 The inventorsll (ibidi, 80826). The next day cells were washed once in ice-cold PBS and fixed in 4% PFA for 10 minutes. Subsequently cells were permeabilized with PBS+0.2% Triton for 10 minutes. Proximity ligation assay using the Duolink® In Situ Red Starter Kit Mouse/Rabbit (Sigma-Aldrich, DUO92101-1KT) was performed according to the manufacturer's instructions. Briefly primary monoclonal antibodies against mouse-AR (Thermo Fisher Scientific, MA5-13426), rabbit-HSP90 (Abcam, ab203085), rabbit-PDCD7 (Abcam, 121258) and rabbit IgG Isotype Control antibody (Thermo Fisher Scientific, 026102) were diluted in Duolink Antibody Diluent (1:50, 1:200, 1:100 and 1:1000). Cells were incubated in the AB solution over night at 4 C. The next day cells were washed twice and incubated for one hour at 37 C in a moisture chamber with PLUS and MINUS PLA probes. Subsequently cells were washed twice and incubated at 37 C (humidity chamber) for 30 min in the ligation mix and 100 minutes in the amplification solution. After two final washes for 10 minutes slides were mounted with DAPI containing media and monitored with a fluorescence microscope (LEICA, DMI4000 B).

Cell Transfection and siRNA-Mediated Knock-Down

### Cells

ON-TARGET plus siRNA SMARTpool siRNAs against U6atac, AR, EZH2, PDCD7, mouse RNPC3 and the Non-targeting (siScrambled) siRNA were purchased from Dharmacon. siRNAs against RNU6atac and RNU12 and the Silencer Select Negative Control were purchased from Thermo Fisher Scientific and siRNA against mouse U6atac was purchased from Ambion. Transfection was performed for the respective timepoints on attached cells using the Lipofectamine™ RNAiMAX Transfection Reagent (Thermo Fisher Scientific, 13778150) to the proportions of 16 pmol of 20 μM siRNA per well.

### Organoids

Before transfection organoids were cultured for 2-3 weeks in human organoid growth medium. Media was removed and organoids were first mechanically dissociated. To obtain single cells organoids were trypsinized in 1 ml TripIE (Thermo Fisher Scientific, 12605036) for 15-18 minutes at 37 C. The reaction was stopped with 1 ml growth media and cells were spun for 5 minutes at 300 g. Subsequently the cells were strained and counted. Per condition one million cells were plated in a 6 well. Lipofectamine™ RNAiMAX complexes were prepared according to the standard Lipofectamine™ RNAiMAX protocol. In short, 5 ul of RNAiMAX reagent and 40 nM of siRNA plus 10% FBS were each diluted in 125 ul Opti-MEMH medium. Both mixes were pooled and incubated for 10 minutes before the siRNA-reagent complex was added to the cells. Cell/siRNA mix was centrifuged at 600 g at 32C for 60 min, and then incubated over night at 37 C. The next day cells were resuspended and collected by centrifugation (300 g, 5 min, RT). The pellet was resuspended in 280 ul Matrigel and the mix was separated into 7 drops that were added into a 6 well. Organoids were grown in human organoid media for 96 h (CTG assay) or seven days (cell counting assay).

RNA Extraction from Cells and qRT-PCR

Cells were harvested for RNA isolation using the ReliaPrep™ miRNA Cell and Tissue Miniprep System (Promega, Z6212). Synthesis of complementary DNAs (cDNAs) using FIREScript RT cDNA Synthesis Kit (Solis BioDyne, 06-15-00200) and real-time reverse transcription PCR (RT-PCR) assays using HOT FIREPol EvaGreen qPCR Mix Plus (Solis BioDyne, 08-24-00020) were performed using and applying the manufacturer protocols. Quantitative real-time PCR was performed on the ViiA 7 system (Applied Biosystems). All quantitative real-time PCR assays were carried out using three technical replicates. Relative quantification of quantitative real-time PCR data used GAPDH, ACTB as housekeeping genes.

### Single Cell Sequencing

Cell counting and viability assessments were conducted using a ViCell XR Cell counter and viability analyzer (Beckman Coulter, BA30273). Thereafter, GEM generation & barcoding, reverse transcription, cDNA amplification and 3′ gene expression library generation steps were all performed according to the Chromium Next GEM Single Cell 3′ Reagent Kits v3.1 User Guide (10× Genomics CG000204 Rev D) with all stipulated 10× Genomics reagents. Generally, 11.8.-27.5 μL of each cell suspension (600-1′400 cells/μL) and 15.7-31.4 μL of nuclease-free water were used for a targeted cell recovery of 10′000 cells. GEM generation was followed by a GEM-reverse transcription incubation, a clean-up step and 10-12 cycles of cDNA amplification. The resulting cDNA was evaluated for quantity and quality using a Thermo Fisher Scientific Qubit 4.0 fluorometer with the Qubit dsDNA HS Assay Kit (Thermo Fisher Scientific, Q32854) and an Advanced Analytical Fragment Analyzer System using a Fragment Analyzer NGS Fragment Kit (Agilent, DNF-473), respectively. Thereafter, 3′ gene expression libraries were constructed using a sample index PCR step of 11-12 cycles. The generated cDNA libraries were tested for quantity and quality using fluorometry and capillary electrophoresis as described above. The cDNA libraries were pooled and sequenced with a loading concentration of 300 μM (150 μM in runs using XP workflow), paired end and single indexed, on an illumina NovaSeq 6000 sequencer using a NovaSeq 6000 S2 Reagent Kit v1.5 (100 cycles; illumina 20028316) and two NovaSeq 6000 S4 Reagent Kits v1.5 (200 cycles; illumina 20028313). The read set-up was as follows: read 1: 28 cycles, i7 index: 8 cycles, i5: 0 cycles and read 2: 91 cycles. The quality of the sequencing runs was assessed using illumina Sequencing Analysis Viewer (illumina version 2.4.7) and all base call files were demultiplexed and converted into FASTQ files using illumina bc12fastq conversion software v2.20. All steps were performed at the Next Generation Sequencing Platform, University of Bern.

### Bulk RNA Sequencing

Total RNA was extracted from LNCaP, C4-2, 22Rv1 and PM154 cells treated for 96 h with siU6atac or siScrambled. The recommended DNase treatment was included. The quantity and quality of the extracted RNA was assessed using a Thermo Fisher Scientific Qubit 4.0 fluorometer with the Qubit RNA BR Assay Kit (Thermo Fisher Scientific, Q10211) and an Advanced Analytical Fragment Analyzer System using a Fragment Analyzer RNA Kit (Agilent, DNF-471), respectively. Thereafter cDNA libraries were generated using an illumina Stranded Total RNA Prep, Ligation with Ribo-Zero Plus (illumina, 20040529) in combination with IDT for Illumina RNA UD Indexes Sets A and B (Illumina, 20040553 and 20040554, respectively). The illumina protocol was followed exactly with the recommended input of 100 ng total RNA. The quantity and quality of the generated NGS libraries were evaluated using a Thermo Fisher Scientific Qubit 4.0 fluorometer with the Qubit dsDNA HS Assay Kit (Thermo Fisher Scientific, Q32854) and an Advanced Analytical Fragment Analyzer System using a Fragment Analyzer NGS Fragment Kit (Agilent, DNF-473), respectively. As a further quality control step, prior to NovaSeq 6000 sequencing, the pooled cDNA library pool underwent paired end sequencing using iSeq 100 i1 reagent v2, 300 cycles (illumina, 20040760) on an iSeq 100 sequencer. The library pool was re-pooled to ensure an equal number of reads/library and then paired end sequenced using a NovaSeq 6000 S4 reagent kits v1.5, 300 cycles (illumina, 20028312) on an Illumina NovaSeq 6000 instrument. The quality of the sequencing runs was assessed using illumina Sequencing Analysis Viewer (illumina version 2.4.7) and all base call files were demultiplexed and converted into FASTQ files using illumina bcl2fastq conversion software v2.20. The average number of reads/libraries was 82 million. The RNA quality-control assessments, generation of libraries and sequencing runs were performed at the Next Generation Sequencing Platform, University of Bern, Switzerland.

### U6atac In Situ Hybridization

mRNA ISH was performed by automated staining using Bond RX (Leica Biosystems) and Basescope® technology (Advanced Cell Diagnostics, Hayward, CA, USA). All slides were dewaxed in Bond dewax solution (product code AR9222, Leica Biosystems) and heat-induced epitope retrieval at pH 9 in Tris buffer based (code AR9640, Leica Biosystems) for 15 min at 950 and Protease treatment for 5 min. The following probes from RNAscope 2.5 LS (Advanced Cell Diagnostics) were used: BaseScope™ LS Probe—BA-Hs-RNU6ATAC-1zz-st-C1ref 1039918, PPIB-1zz ref 710178 and DapB-1zz ref 701028, were used as positive and negative control respectively. Probe efficiency was tested using U6atac overexpressing C4-2 cells (5 million) of which 50% were treated with siU6atac RNA.

All probes were incubated at 370 for 120 min. Basescope™ 2.5 LS Assay (Ref 323600, Advanced Cell Diagnostics) was used as pre-amplification system. Subsequent the reaction was visualized using Fast red as red chromogen (Bond polymer Refine Red detection, Leica Biosystems, Ref DS9390) for 20 min. Finally, the samples were counterstained with Haematoxylin, air dried and mounted with Aquatex (Merck). Slides were scanned and photographed using Pannoramic 250 (3DHistech). U6atac intensity was scored manually by a pathologist (Mark Rubin) blinded to the clinical data, using the digital online TMA scoring tool Scorenado (University of Bern, Switzerland) especially developed for TMA scoring on de-arrayed spots.

For the analysis of TMA data, samples annotated as ‘center’ were used. U6ATAC score was calculated by multiplying the percentage of positive cells by the intensity. The sample with the highest score was used where more than one value was recorded for a block. Comparisons between groups were carried out using Wilcox test.

325 primary, 25 primary with metastatic potential and 32 metastatic samples, from 24 patients were used for the comparison of U6ATAC expression in PCa and PCBM.

### Flow Cytometry

C4-2 and PM154 cells were seeded in a 6 well (500 000/well) and transfected with siU6atac or siScrambled RNA for 72 and 96 hours as previously described. Flow Cytometry cell cycle analysis was performed using the Click-iT™ EdU Alexa Fluor™ 488 Flow Cytometry Assay Kit (Thermo Fisher Scientific, C10420). Briefly EdU (10 uM) was added into the media and cells were incubated for one hour at 37 C. cells were washed with 1% BSA in PBS and fixed in 100 ul Click-iT fixative for 15 minutes. After three additional washing step cells were permeabilized for 15 minutes in 100 ul 1×Click-iT saponin based reagent. Click-iT reaction cocktail was prepared according to manufacturer's instructions and 500 ul reaction mix/condition were incubated for 30 min with the cells at room temperature. Cells were washed and resuspended in 500 ul saponin-based permeabilization buffer. Hoechst (1 ug/ml) was added 20 minutes prior analysis to the reaction mix. Cells were analyzed using the FACSDiva Software on a BD LSR II Flow Cytometer (BD Biosciences) in the FACSIab Core facility of the University of Bern. Data was further quantified with FlowJo 10.7.1. Values were calculated as fold-change as compared to siScrambled treated controls.

### Immunoblotting

Cells were lysed in GST-Fish buffer (10% (v/v) Glycerol, 50 mM Tris-HCl pH7.4, 100 mM NaCl, 1% (v/v) Nonidet P-40, 2 mM MgCl₂, 1 mM PMSF) with freshly added protease and phosphatase inhibitors. Total protein concentration was measured using the Pierce BCA Protein Assay Kit (Thermo Fisher Scientific). 50 ug protein samples were resolved 4-15% Mini-Protean TGX gels (BioRad, 456-1084) in SDS-PAGE and transferred to nitrocellulose membranes using the iBlot2 system (Thermo Fisher, 1B23001). Blots were blocked for 1 hour at room temperature in 5% milk/TBST or BSA/TBST and incubated overnight at 4° C. with primary antibodies which were dissolved in 5% BSA/TBST buffer. After 3 washes, the membrane was incubated with secondary antibody conjugated to horseradish peroxidase for 1 h at room temperature. After 3 washes, signal was visualized by chemiluminescence using the Luminata Forte substrate (Thermo Fisher Scientific, WBLUF0100) for strong antibodies and The inventorssternBright Sirius-HRP Substrate (Witec AG, K-12043-D10) for weak antibodies. Images were acquired with the FUSION FX7 EDGE Imaging System (Witec AG).

### Luciferase Reporter Assay

CMV-luc2CP/ARE (major intron splicing reporter), CMV-luc2CP (empty vector backbone control) and luc1CFH4 (minor splicing intron reporter) were a kind gift from Dr. Gideon Dreyfuss (University of Pennsylvania). DNA was amplified via chemical transformation of One Shot Mach1 T1 Phage-Resistant Chemically Competent E. coli cells (Invitrogen, C862003) and Sanger sequenced.

To determine the minor/major intron splicing rate cells were seeded in white 96 well plate (Huberlab, 7.655 098) (8000/well) and treated according to assay conditions. 24 hours prior analysis cells of each condition were co-transfected with each reporter plasmid and the empty vector backbone plasmid. In short 1.5 ul of P3000 reagent plus 0.5 ug of DNA and 1.5 ul Lipofectamine P300 were each diluted in 25 ul Opti-MEMH medium. Both mixes were pooled and incubated for 20 minutes before the solution was added to the cells. Luciferase expression was measured with the Dual-Glo® Luciferase Assay System (Promega, E2940): media was removed, 100 ul of PLB were added and cells were frozen for two hours at −20 C. After a one hour shaking step 100 ul of LAR substrate was added and Firefly luciferase expression was measured with a Tecan Infinite M200PRO reader. Values were calculated as x-fold of CMV-luc2CP expression and subsequently as the x-fold of the respective reference control.

### Minigene Reporter Assay

pRSV2-p120-AmpR XL10 (p120), pCMV7.1 SCN4A frag (hSCN4A) and pLUX hSCN8A-minigene AmpR STbl3 (hSCN8a) were a kind gift from Dr. Mark-David Ruepp (King's College London). DNA was amplified via chemical transformation of One Shot Mach1 T1 Phage-Resistant Chemically Competent E. coli cells (Invitrogen, C862003).

To determine the minigene splice index cells were seeded in a 6-well (350 000/well) and treated according to assay conditions. SiRNA was added 96 hours prior measurement. 72 hours prior measurement cells were transfected with the respective minigene. Briefly 10 ul of P3000 reagent plus 1.5 ug of DNA and 7.5 ul Lipofectamine P3000 were each diluted in 125 ul Opti-MEMH medium. Both mixes were pooled and incubated for 20 minutes before the solution was added to the cells. 48 hours before the measurement media was exchanged for media with 10% charcoal stripped FBS (Gibco, A3382101) and 24 hours prior measurement 100 nM DHT was added to the respective condition. qRT-PCR was performed to verify the KD and to determine the splice index of each minigene. The minigene splice index was calculated by forming the ratio of normalized mRNA levels of cells transfected with the minigene versus mRNA levels of WT cells to consider the transfection efficiency. Subsequently the values corresponding to the spliced minigene were divided by the values corresponding to the unspliced minigene.

### Cell Growth Experiments

### Viability

Cells were seeded in a 6 well (400 000) and treated according to assay conditions over night. Cells were then seeded in Poly-L-Lysine coated 96-well plates (8000 cells/well, n=3 per condition) and WCM154 cells were seeded in a collagen-coated 96-well plates (5000 cells/well, n=3 per condition). Remaining cells were used for U6atac KD control via qRT-PCR. Cell viability was determined after 24, 48, 72, and 96 h with a Tecan Infinite M200PRO reader using the CellTiter-Glo® Luminescent Cell Viability Assay according to manufacturer's directions (Promega, G9243). Viability values were calculated as x-fold of cells transfected with siRNA for 0 h. Cell confluence (n=4 per condition) was determined using the Incucyte S3 instrument and the IncuCyte S3 2018B software (Essen Bioscience, Germany). Values were calculated as x-fold of timepoint 0 and then as fold-change in confluency as compared to siScrambled treated controls.

### Organoids

Organoids were transfected with siRNA as described previously. The following day 160000 cells were resuspended in 320 ul Matrigel and drops of 40 ul (one drop/well, four timepoints, n=2) were plated in a suspension 48-well plate (Huberlab, 7.677 102). Remaining cells were plated in a 6-well for q RT-PCR U6atac KD control. The 24-well plate was incubated for three minutes in 37C and for 20 minutes upside down in 37 C. Subsequently 500 ul of organoid media were added and viability was measured using the CellTiter-Glo 3D Cell Viability Assay (Promega, G9683).

### Co-Immunoprecipitation

For the co-immunoprecipitation (co-IP), cytosolic fractions of LNCaP-AR cells were isolated using the Universal ColP Kit (Active Motif, 54002). Chromatin of the cytosolic fraction was mechanically sheared using a Dounce homogenizer Fisher Scientific, 11898502). Cytosolic membrane and debris were pelleted by centrifugation and protein concentration of the cleared lysate was determined with the Pierce BCA Protein Assay Kit (Thermo Fisher Scientific, 23227). One microgram of the rabbit anti-PDCD7 (ab131258, Abcam) and rabbit anti-AR (ab133273, Abcam) antibodies and 1 μg of rabbit IgG Isotype Control antibody (Thermo Fisher Scientific, 026102) were incubated with 1 mg protein supernatant overnight at 4° C. with gentle rotation. The following morning, 30 μl of Protein G Magnetic Beads (Active Motif were washed twice with 500 μl ColP buffer and incubated with Antibody-containing lysate for 2 hours at 4° C. with gentle rotation. Bead-bound PDCD7 or AR complexes were washed twice with ColP buffer and subsequently twice with a buffer containing 150 mM NaCl, 50 mM Tris-HCL (pH 8) and Protease and Phosphatase inhibitors. Washing procedure was executed at 4° C. with gentle rotation. Bead-bound protein, supernatant and Input controls were reduced and denatured in 40 μl Laemmli buffer containing DTT through boiling for 5 min at 95° C. Magnetic beads were removed from solution using Magnetized Pipette Racks (Thermo Fisher Scientific, 11757325) and 20 μl of reduce protein was loaded on an SDS-PAGE gel with subsequent immunoblotting using iBlot (Life Technologies). Membranes were blocked in 5% BSA solution and then incubated over night with respective antibodies against targets of interest: AR, PDCD7, AR45 (Androgen Receptor Antibody (Carboxy-terminal Antigen), Cell Signaling Technologies, 54653S), ZRSR2 (Abcam, ab223062). Protein signal was detected using HRP-labeled native anti-rabbit IgG antibody (CST, #5127) and Luminata Forte substrate (Thermo Fisher Scientific, WBLUF0100) using the FUSION FX7 EDGE Imaging System (Witec AG).

### Protein-Protein Interaction Network Analysis

Assuming that cancer genes perturb a large molecular network through their interactions with other genes, distance to MIGs in a protein-protein interaction network of 160,881 interactions between 15,366 human proteins as of the HINT database was determined (Das, J., et al., ibid). In particular, a MIG that directly interacts with a cancer-causing gene is a distance d=1 away, while a protein that is separated by a protein in between is a distance d=2 away from a cancer-genes in question. To find these interaction distances, a list of 403 cancer-causing genes from 186 different cancer types as of the Cancer Genome Interpreter database was considered (https://www.cancergenomeinterpreter.org) in the underlying protein-protein interaction network of 160,881 interactions between 15,366 human proteins as of the HINT database²¹. A profile of the numbers of MIGs with a given distance d away from each cancer gene, N_{d}^{MIG} (d=1, . . . , 5) was obtained. To assess if the presence of MIGs in the vicinity of cancer genes is significant, sets of 542 MIGs found in the underlying interaction networks were randomly sampled. In particular, the corresponding distances of cancer genes to these randomly sampled MIGs was measured and a profile of numbers of random MIGs with a given distance d away from cancer genes N_{d}^{r,MIG} (d=1, . . . , 5) was obtained. The enrichment of MIGs is defined a distance d away from cancer genes as

$${E}_{d}^{MIG} = {lg}_{2} ( \frac{{N}_{d}^{MIG}}{{N}_{d}^{r , MIG}} ) ,$$

with average E over 100,000 random samples of MIGs.

To find a tight-knit web of MIGs and cancer genes, such direct interactions of cancer genes and MIGs were distracted in the underlying human protein-protein interaction network and determined the size of the largest connected subnetwork S. Randomly sampling MIGs N=100,000 times. The sizes of the largest subnetworks was calculated using these random sets, Sᵣ, and the significance of S by

$$P = \frac{\# ( {S}_{r} > S )}{N}$$

was determined.

### Principal Component Analysis

MIG expression data from 3 sources: prostate samples from GTEx (healthy prostate tissue), prostate cancer samples from TCGA (primary prostate cancer samples), and prostate cancer samples from SU2C (advanced prostate cancer) was merged. Gene expression values were normalized following the protocol adopted by GTEx (and detailed in the inventors' pan-cancer analysis). PCA analysis on this normalized gene expression matrix was carried out, thereby enabling visualization of the resultant data as the projections onto the space spanned by the first 2 PCs. Notably, this visualization appears to capture the progression from these 3 broad phases of prostate cancer progression, from healthy tissue in GTEx (at lower ends of the first PC) to advanced stages in SU2C (at higher ends of the first PC).

### Quantitative Comparisons Between MIG- and Non-MIG-Based Gene Expression Clustering

The Silhouette coefficient⁶⁴ was used in order to characterize the relative performance of gene expression clustering for gene sets containing different relative abundances of MIGs and non-MIGs. The Silhouette coefficient provides an objective metric for measuring what is visually discerned to be structure (or any lack thereof) in a given heatmap. This coefficient constitutes an unsupervised approach to provide a score ranging −1 and 1, with scores closer to 1 indicative of well-defined and dense clustering (i.e., more meaningful structure in a given heatmap). This coefficient quantifies how similar a given data point (i.e., sample) is to its own cluster relative to different clusters. In the inventors' study, a data point consists of a N-length vector, where N is the number of distinct samples in an expression matrix, and the number of distinct clusters is pre-defined to be n_cluster=9 in the inventors' pan-cancer analysis, since this analysis was carried out on a dataset of 9 distinct cancer types. The Silhouette coefficient has also been adopted for similar purposes in previous studies (Belorkar, A. et al., BMC Bioinformatics 2016, 17 (Suppl 17), 540; Van Laere, S. J. et al., Clin Cancer Res 2013, 19 (17), 4685-96; Zhao, S. et al., Biol Proced Online 2018, 20, 5; Lovmar, L. et al., BMC Genomics 2005, 6, 35).

Prior to calculating the Silhouette coefficient, an agglomerative clustering on a normalized gene expression matrix was performed. Gene expression values from 9 cancer types (Biliary-, Breast-, ColoRect-, Lung-, Ovary-, Panc-, Prost-, Stomach-, Thy-adenocarcinoma) totaling N=444 samples were taken from PCAWG, and the gene expression normalization was performed using the same approach as that adopted by GTEx (PMID: 29022597). Briefly, the entire gene expression matrix was normalized using quantile normalization. Then, inverse quantile normalization was applied to this quantile-normalized matrix in order to map to a standard normal (this also enabled the inventors to remove outliers). For the PCa progression analysis, gene expression values from GTEx, TCGA, and SU2C were merged into one expression matrix. Briefly the pre-normalized expression matrix was normalized using quantile normalization, followed by inverse quantile normalization to map to a standard normal distribution; this step also removed outlier genes. This normalization scheme was the same as that adopted previously by GTEx (PMID: 29022597). Using this normalized gene expression matrix, clustering was performed by using hierarchical clustering by employing the Euclidean metric and Ward linkage.

The entire analysis was run using different expression matrices with varying fractions of MIG genes, and the results are plotted in FIG. 1F and G. Each box in this plot (for instance, the right-most box, which represents 100% non-MIG sets) corresponds to a 1000-length simulation in which non-MIGs are randomly sampled from among all non-MIGs in the genome. Thus, for the case of the right-most box, 1000 random sets are sampled, each of which has a composition of 100% non-MIGs. The P-value is based on a two-sided t-test in which each sample value is taken to be the difference between the Silhouette coefficient of one of the 1000 100% non-MIG samples and the corresponding Silhouette coefficient when only MIGs are used for clustering (i.e., the Silhouette coefficient corresponding to 0% non-MIGs).

### MSI Analysis

Primary tumor RNA-seq patient samples from The Cancer Genome Atlas (TCGA) were randomly queried using the GenomicDataCommons package in R (https://github.com/Bioconductor/GenomicDataCommons). The minor intron retention pipeline developed by Olthof et. al. (https://github.com/amolthof/minor-intron-retention) was run on the queried TCGA samples from the following cohorts: breast invasive carcinoma (BRCA; N=20), cholangiocarcinoma (CHOL; N=20), colon adenocarcinoma (COAD; N=20), lung adenocarcinoma (LUAD; N=20), ovarian serous cystadenocarcinoma (OV; N=20), pancreatic adenocarcinoma (PAAD; N=20), prostate adenocarcinoma (PRAD; N=16), and thyroid carcinoma (THCA; N=20). For the prostate cancer analyses, the following additional samples from the Stand Up to Cancer dataset were analyzed: androgen receptor (AR; N=20) and neuroendocrine (NE; N=22). NE samples were samples which had an NEPC score of greater than 0.4 while AR samples had an NEPC score of less than or equal to 0.4. Prostate cancer samples from the Genome-Tissue Expression Portal were analyzed as well (GTEX; N=20). A Kruskal-Wallis with post-hoc Dunn's Test was performed between the TCGA cohorts and the prostate cohorts. A heatmap was generated with the gplots package in R with the default clustering method for the pan-cancer TCGA cohorts and the prostate cohorts across the MIGs (https://CRAN_R-pr2ect.org/package=gplots). A GO Enrichment Analysis was performed on the genes that clustered for each cancer cohort, grouping genes with a mean MSI value in the ranges of 0, 0 to 0.04, 0.04 to 0.75, and 0.75 to 1.

### RNU11 Quantification According to Gleason Score

Gene-expression data of primary prostate cancer specimen was retrieved from The Cancer Genome Atlas (TCGA) in form of raw-counts. Sequencing reads were aligned to the human reference genome (hg38) using STAR (Lovmar, L. et al., BMC Genomics 2005, 6, 35). Gene-expression was quantified at gene-level using Gencode annotations (v29) (Harrow, J. et al., Genome Res 2012, 22 (9), 1760-74). Subsequent analysis and library-size normalization were performed using edgeR pipeline (Chen, Y. et al., F1000Res 2016, 5, 1438). RNU11 mapping reads were identified in 23 out of 497 samples (5%) which reflects the difficulty of capturing this gene product using canonical PolyA+ sequencing techniques. Nonetheless, a clear association between Gleason score and RNU11 mRNA expression in these 23 samples was identified. Significance was assessed using non-parametric Wilcoxon Test.

### Single-Cell RNAseq

### Single-Cell Preprocessing and Quality Control:

Cell ranger analysis pipeline v6.0.1 was used to align reads to the human genome reference sequence (GRCh38) and generate a gene-cell matrix from these data. The gene expression matrix was analyzed using Seurat 4.0.3 (https://github.com/satijalab/seurat). The inventors removed low quality cells and multiplets by excluding genes detected in less than 5 cells and by discarding cells with more than 10000 fewer than 1000 detected genes. Cells containing mitochondrial gene counts greater than 25% were also removed.

UMI counts were normalized with the NormalizeData Seurat function using the LogNormalize normalization method with default parameters (10000 scale.factor).

### Cell Cycle Phase Classification and Cell Scores:

Prediction of cell cycle phase for each cell was performed using Seurat CellCycleScoring function. A score was computed and a cell phase (G2/M, S and G1) was assigned to the cell as described previously (Tirosh, I. et al., Science 2016, 352 (6282), 189-196). Fisher's exact test was performed to check whether the slU6 cells have significantly a different number of cells than the Scr in G1 or S phase using the R fisher.test function.

The inventors used Seurat AddModuleScore function to evaluate the degree to which individual cells express a certain pre-defined gene set. The inventors defined scores to estimate the activities of prostate AR pathway, and EMT state, as described previously (Dong, B. et al., Communications Biology 2020, 3 (1), 778). The AR pathway gene set included AR, KLK3, KLK2, FKBP5, TMPRSS2, FOXA1, GATA2, SLC45A3 and EMT state CDH2, CDH11, FN1, VIM, TWIST1, SNAI1, ZEB1, ZEB2 and DCN. Violin plots were drawn using Seurat and p-values were calculated using Wilcoxon test (Hadley Wickham, ggplot2: Elegant Graphics for Data Analysis 2016).

### Multiple Datasets Integration and Batch Correcting:

For merging multiple datasets and minimizing the batch effect between them, the inventors integrated the inventors' 6 samples (3 replicates SCR and 3 replicates siU6) for each cell line following the procedure of Seurat v4.0.3 (Stuart, T. et al., Cell 2019, 177 (7), 1888-1902.e21).

Briefly, the inventors selected the most variable genes for each dataset using the FindVariableFeatures function (selection.method=“vst”) and ranked them according to the number of datasets in which they were independently identified as highly variable. The 2000 most variable genes were thus integrated by merging pairs of datasets according to a given distance.

Integration anchors, representing two cells that are predicted to originate from a common biological state in both datasets using a Canonical Correlation Analysis (CCA), were done using the FindIntegrationAnchors function. The expression of the target dataset was corrected using the difference in expression between the two expression vectors for each pair of anchor cells. This step was performed using the IntegrateData function. This process resulted in an expression matrix containing the batch-effect-corrected expression for the 2000 selected genes for all cells from the 6 samples for each cell line.

### Dimension Reduction and Clustering:

A PCA was performed on the scaled data using RunPCA Seurat function (npcs=30). Uniform Manifold Approximation and Projection (UMAP), a nonlinear dimension reduction method, was run using RunUMAP Seurat package function in order to embed cells in a 2-dimensional space. A K-nearest neighbor graph (KNN) based on the Euclidean distance in PCA space was constructed to cluster the cells with the Louvain algorithm (resolution=0.2) using the FindNeighbors and FindClusters Seurat functions. The inventors selected the optimal clustering resolution using the clustree R package (v0.4.3) (Zappia, L., et al., GigaScience 2018, 7 (7)). Barplots were performed using dittoSeq R package (Bunis, D. G. et al., Bioinformatics 2020, 36 (22-23), 5535-5536).

### Differential Gene Expression Analysis:

Differentially expressed genes (DEGs) were identified between the different clusters using the FindAllMarkers function from the Seurat package (one-tailed Wilcoxon rank-sum test, p values adjusted for multiple testing using the Bonferroni correction). To compute DEGs, all genes were tested provided they were expressed in at least 25% of cells in either of the two compared populations, and the expression difference on a natural log scale was at least 0.25. The heatmap was produced using the DoHeatmap Seurat function by selecting the top five genes for each cluster.

### RNAseq

### Gene Expression:

Paired-end, total RNA reads for each replicate (N=4) were mapped to the mm10 genome via Hisat2 (Kim et al., 2015). Reads mapping to multiple locations were removed. Gene expression values were calculated using IsoEM2 (Mandric et al., 2017). Differential gene expression was calculated by IsoDE2 (Mandric et al., 2017), which uses 200 rounds of iterative bootstrapping to produce a 95% confidence interval for the expression of each gene, then statistically compares these values between experimental conditions. A threshold of log 2FC≥1, P<0.01 for upregulation, and log 2FC <−1, P s 0.01 for downregulation was employed.

### Minor Intron Retention:

The inventors report here minor intron retention as a mis-splicing index through the methodology described in Olthof et al., 2019. Briefly, uniquely mapped reads from the region of interest around minor introns (from two exons upstream to two exons downstream) were extracted. The mis-splicing index was then calculated by summing reads that map to the 5′ splice site and 3′ splice site of a minor intron, divided by the sum of reads that map to the 5′ splice site, 3′ splice site, and 2× canonically spliced reads. The inventors only considered introns that pass the inventors' filtering criteria, which requires >4 exon-intron boundary reads, >1 read mapping to both the 5′ splice site and 3′ splice site, and >95% intron coverage, in all replicates of a condition as retained. Statistically significant global minor intron retention was determined using a Kruskal-Wallis test with post-hoc Dunn's test (P≤0.05). Determination of individual MIGs with significantly elevated minor intron retention was calculated using a two-tailed student's T-test (P s 0.05).

### Alternative Splicing:

The inventors employed the methodology reported by Olthof et al., 2017 for the inventors' alternative splicing analysis. Briefly, the inventors used BEDTools to classify differential 5′ splice site and 3′ splice site usage around the region of interest for all minor introns and binned them into one of 8 categories (FIG. 3D). The inventors then calculated a mis-splicing index by quantifying the number of AS reads divided by the sum of AS reads and canonically spliced reads per category per sample. The inventors used a filtering criteria wherein the inventors only considered introns to have AS if the average mis-splicing index for all replicates for each condition was >10%. Additionally, the inventors normalized the number of reads supporting an AS event by the total sequencing depth. As such, the inventors only included AS events with >1 read per 3 million uniquely mapped reads for analysis. Determination of individual MIGs with significantly elevated AS was calculated using a two-tailed student's T-test (P s 0.05).

### DAVID Analysis:

Gene lists were submitted to DAVID for gene ontology (GO) enrichment. The inventors considered only GO Terms with Benjamini-Hochberg adjusted P-value s 0.05 as significant.

### STRING and Ingenuity Pathway Analysis:

For LNCaP. C4-2, and 22Rv1, overlapping MIGs with significantly elevated minor intron retention that were also found to be associated with prostate cancer-causing genes were grouped with overlapping protein coding genes with significant downregulation. This list was submitted to STRING under the default parameters to obtain the gene interaction network. Subsequently, the same list was submitted to IPA as a core analysis using default parameters. All reported biological networks and pathways from IPA were significant using a Benjamini-Hochberg adjusted P-value s 0.05 cut-off. The same analysis was performed for PM154 alone.

### Principal Component Analysis:

Principal component analyses were performed using clustvis (Metsalu & Vilo, 2015) with default parameters. Ellipses show 95% confidence interval.

### GSEA Analysis:

To perform gene set enrichment analysis RNAseq data was pre-ranked using the metric: log 10(Pvalue)/sign (log FC). GSEA was performed using the GSEA v.4.0.3 software. Hallmark gene sets, obtained from the GSEA website (www.broadinstitute.org/Qsea) was used for enrichment of siU6atac related pathway genes. Dotplot was used to visualize the most significant enriched terms. Normalised enrichment score (NES) and False discovery rate (FDR) were applied to sort siU6atac pathway enrichment after gene set permutations were performed 1000 times for the analysis.

### MassSpec

MS data was interpreted with MaxQuant (version 1.6.1.0) against a SwissProt human database (release 2019_07) using the default MaxQuant settings, allowed mass deviation for precursor ions of 10 ppm for the first search and maximum peptide mass of 5500 Da; match between runs with a matching time window of 0.7 min was activated, but prevented between different groups of replicates by the use of non-consecutive fractions. Furthermore, the four cell lines were treated as different parameter groups and normalized independently. Settings that differed from the default also included: strict trypsin cleavage rule allowing for 3 missed cleavages, fixed carbamidomethylation of cysteines, variable oxidation of methionines and acetylation of protein N-termini.

Protein intensities are reported as MaxQuant's Label Free Quantification (LFQ) values, as well as Top3 values (sum of the intensities of the three most intense peptides); for the latter, variance stabilization was used for the peptide normalization. Missing peptide intensities were imputed in the following manner, provided there was at least one identification in the group: two missing values in a group of replicates would be replaced by draws from a Gaussian distribution of width 0.3×sample standard deviation centred at the sample distribution mean minus 1.8× the sample standard deviation, whereas a single missing value per group would be replaced following the Maximum Likelihood Estlimation (MLE) method. Imputation at protein level for both LFQ and Top3 values was performed if there were at least two measured intensities in at least one group of replicates; missing values in this case were drawn from a Gaussian distribution of width 0.3× sample standard deviation centered at the sample distribution mean minus 2.5× the sample standard deviation. Differential expression tests were performed using the moderated t-test empirical Bayes (R function EBayes from the limma package version 3.40.6) on imputed LFQ and Top3 protein intensities. The Benjamini and Hochberg method was further applied to correct for multiple testing. The criterion for statistically significant differential expression is that the maximum adjusted p-value for large fold changes is 0.05, and that this maximum decrease asymptotically to 0 as the log 2 fold change of 1 is approached (with a curve parameter of one time the overall standard deviation). The protein imputation step was repeated 20× so as to be able to flag those proteins that are persistently significantly differentially expressed throughout the cycles.

Imputed iTop3 was used to calculate relative protein abundances. Differential expression was calculated using the Empirical Bayes test. Protein upregulation and downregulation was determined by setting a threshold of Benjamini-Hochberg adjusted P-value <0.05, log 2FC >1 or <−1, respectively.

### REFERENCES

Bluemn, E. G.; Coleman, I. M.; Lucas, J. M.; Coleman, R. T.; Hernandez-Lopez, S.; Tharakan, R.; Bianchi-Frias, D.; Dumpit, R. F.; Kaipainen, A.; Corella, A. N.; Yang, Y. C.; Nyquist, M. D.; Mostaghel, E.; Hsieh, A. C.; Zhang, X.; Corey, E.; Brown, L. G.; Nguyen, H. M.; Pienta, K.; Ittmann, M.; Schweizer, M.; True, L. D.; Wise, D.; Rennie, P. S.; Vessella, R. L.; Morrissey, C.; Nelson, P. S., Androgen Receptor Pathway-Independent Prostate Cancer Is Sustained through FGF Signaling. Cancer Cell 2017, 32 (4), 474-489.e6.
Das, J.; Yu, H., HINT: High-quality protein interactomes and their applications in understanding human disease. BMC Syst Biol 2012, 6, 92.
Tamborero, D.; Rubio-Perez, C.; Deu-Pons, J.; Schroeder, M. P.; Vivancos, A.; Rovira, A.; Tusquets, I.; Albanell, J.; Rodon, J.; Tabernero, J.; de Torres, C.; Dienstmann, R.; Gonzalez-Perez, A.; Lopez-Bigas, N., Cancer Genome Interpreter annotates the biological and clinical relevance of tumor alterations. Genome Medicine 2018, 10 (1), 25.=
Dong, B.; Miao, J.; Wang, Y.; Luo, W.; Ji, Z.; Lai, H.; Zhang, M.; Cheng, X.; Wang, J.; Fang, Y.; Zhu, H. H.; Chua, C. W.; Fan, L.; Zhu, Y.; Pan, J.; Wang, J.; Xue, W.; Gao, W.-Q., Single-cell analysis supports a luminal-neuroendocrine transdifferentiation in human prostate cancer. Communications Biology 2020, 3 (1), 778.
Ku, S. Y.; Rosario, S.; Wang, Y.; Mu, P.; Seshadri, M.; Goodrich, Z. W.; Goodrich, M. M.; Labbe, D. P.; Gomez, E. C.; Wang, J.; Long, H. W.; Xu, B.; Brown, M.; Loda, M.; Sawyers, C. L.; Ellis, L.; Goodrich, D. W., Rbl and Trp53 cooperate to suppress prostate cancer lineage plasticity, metastasis, and antiandrogen resistance. Science 2017, 355 (6320), 78-83.
Bai, Y.; Zhang, Z.; Cheng, L.; Wang, R.; Chen, X.; Kong, Y.; Feng, F.; Ahmad, N.; Li, L.; Liu, X., Inhibition of enhancer of zeste homolog 2 (EZH2) overcomes enzalutamide resistance in castration-resistant prostate cancer. J Biol Chem 2019, 294 (25), 9911-9923.
Mu, P.; Zhang, Z.; Benelli, M.; Karthaus, W. R.; Hoover, E.; Chen, C. C.; Wongvipat, J.; Ku, S. Y.; Gao, D.; Cao, Z.; Shah, N.; Adams, E. J.; Abida, W.; Watson, P. A.; Prandi, D.; Huang, C. H.; de Stanchina, E.; Lowe, S. W.; Ellis, L.; Beltran, H.; Rubin, M. A.; Goodrich, D. W.; Demichelis, F.; Sawyers, C. L., SOX2 promotes lineage plasticity and antiandrogen resistance in TP53- and RB1-deficient prostate cancer. Science 2017, 355 (6320), 84-88.
Puca, L.; Bareja, R.; Prandi, D.; Shaw, R.; Benelli, M.; Karthaus, W. R.; Hess, J.; Sigouros, M.; Donoghue, A.; Kossai, M.; Gao, D.; Cyrta, J.; Sailer, V.; Vosoughi, A.; Pauli, C.; Churakova, Y.; Cheung, C.; Deonarine, L. D.; McNary, T. J.; Rosati, R.; Tagawa, S. T.; Nanus, D. M.; Mosquera, J. M.; Sawyers, C. L.; Chen, Y.; Inghirami, G.; Rao, R. A.; Grandori, C.; Elemento, O.; Sboner, A.; Demichelis, F.; Rubin, M. A.; Beltran, H., Patient derived organoids to model rare prostate cancer phenotypes. Nat Commun 2018, 9 (1), 2404.
Gao, D.; Vela, I.; Sboner, A.; laquinta, P. J.; Karthaus, W. R.; Gopalan, A.; Dowling, C.; Wanjala, J. N.; Undvall, E. A.; Arora, V. K.; Wongvipat, J.; Kossai, M.; Ramazanoglu, S.; Barboza, L. P.; Di, W.; Cao, Z.; Zhang, Q. F.; Sirota, I.; Ran, L.; MacDonald, T. Y.; Beltran, H.; Mosquera, J. M.; Touijer, K. A.; Scardino, P. T.; Laudone, V. P.; Curtis, K. R.; Rathkopf, D. E.; Morris, M. J.; Danila, D. C.; Slovin, S. F.; Solomon, S. B.; Eastham, J. A.; Chi, P.; Carver, B.; Rubin, M. A.; Scher, H. I.; Clevers, H.; Sawyers, C. L.; Chen, Y., Organoid cultures derived from patients with advanced prostate cancer. Cell 2014, 159 (1), 176-187.
Rodriguez, A.; Gallon, J.; Akhoundova, D.; Maletti, S.; Ferguson, A.; Cyrta, J.; Amstutz, U.; Garofoli, A.; Paradiso, V.; Tomlins, S. A.; Hewer, E.; Genitsch, V.; Fleischmann, A.; Rushing, E. J.; Grobholz, R.; Fischer, I.; Jochum, W.; Cathomas, G.; Bubendorf, L.; Moch, H.; Ng, C. K. Y.; Sommer, S. G.; Piscuoglio, S.; Rubin, M. A., The Genomic Landscape of Prostate Cancer Brain Metastases. bioRxiv 2020, 2020.05.12.092296.
Briganti, A.; Spahn, M.; Joniau, S.; Gontero, P.; Bianchi, M.; Kneitz, B.; Chun, F. K.; Sun, M.; Graefen, M.; Abdollah, F.; Marchioro, G.; Frohenberg, D.; Giona, S.; Frea, B.; Karakiewicz, P. I.; Montorsi, F.; Van Poppel, H.; Jeffrey Karnes, R., Impact of age and comorbidities on long-term survival of patients with high-risk prostate cancer treated with radical prostatectomy: a multi-institutional competing-risks analysis. Eur Urol 2013, 63 (4), 693-701.
Tosco, L.; Laenen, A.; Briganti, A.; Gontero, P.; Karnes, R. J.; Bastian, P. J.; Chlosta, P.; Claessens, F.; Chun, F. K.; Everaerts, W.; Gratzke, C.; Albersen, M.; Graefen, M.; Kneitz, B.; Marchioro, G.; Salas, R. S.; Tombal, B.; Van den Broeck, T.; Van Der Poel, H.; Walz, J.; De Meerleer, G.; Bossi, A.; Haustermans, K.; Van Poppel, H.; Spahn, M.; Joniau, S., The EMPaCT Classifier: A Validated Tool to Predict Postoperative Prostate Cancer-related Death Using Competing-risk Analysis. Eur Urol Focus 2018, 4 (3), 369-375.
Chys, B.; Devos, G.; Everaerts, W.; Albersen, M.; Moris, L.; Claessens, F.; De Meerleer, G.; Haustermans, K.; Briganti, A.; Chlosta, P.; Gontero, P.; Graefen, M.; Gratzke, C.; Karnes, R. J.; Kneitz, B.; Marchioro, G.; Salas, R. S.; Spahn, M.; Tombal, B.; Van Der Poel, H.; Walz, J.; Van Poppel, H.; Joniau, S., Preoperative Risk-Stratification of High-Risk Prostate Cancer: A Multicenter Analysis. Front Oncol 2020, 10, 246.
Dawson, H.; Christe, L.; Eichmann, M.; Reinhard, S.; Zlobec, I.; Blank, A.; Lugli, A., Tumour budding/T cell infiltrates in colorectal cancer: proposal of a novel combined score. Histopathology 2020, 76 (4), 572-580.
Belorkar, A.; Wong, L., GFS: fuzzy preprocessing for effective gene expression analysis. BMC Bioinformatics 2016, 17 (Suppl 17), 540.
Van Laere, S. J.; Ueno, N. T.; Finetti, P.; Vermeulen, P.; Lucci, A.; Robertson, F. M.; Marsan, M.; Iwamoto, T.; Krishnamurthy, S.; Masuda, H.; van Dam, P.; Woodward, W. A.; Viens, P.; Cristofanilli, M.; Birnbaum, D.; Dirix, L.; Reuben, J. M.; Bertucci, F., Uncovering the molecular secrets of inflammatory breast cancer biology: an integrated analysis of three distinct affymetrix gene expression datasets. Clin Cancer Res 2013, 19 (17), 4685-96.
Zhao, S.; Sun, J.; Shimizu, K.; Kadota, K., Silhouette Scores for Arbitrary Defined Groups in Gene Expression Data and Insights into Differential Expression Results. Biol Proced Online 2018, 20, 5.
Lovmar, L.; Ahlford, A.; Jonsson, M.; Syvsnen, A. C., Silhouette scores for assessment of SNP genotype clusters. BMC Genomics 2005, 6, 35.
Dobin, A.; Davis, C. A.; Schlesinger, F.; Drenkow, J.; Zaleski, C.; Jha, S.; Batut, P.; Chaisson, M.; Gingeras, T. R., STAR: ultrafast universal RNA-seq aligner. Bioinformatics 2013, 29 (1), 15-21.
Harrow, J.; Frankish, A.; Gonzalez, J. M.; Tapanari, E.; Diekhans, M.; Kokocinski, F.; Aken, B. L.; Barrell, D.; Zadissa, A.; Searle, S.; Barnes, I.; Bignell, A.; Boychenko, V.; Hunt, T.; Kay, M.; Mukherjee, G.; Rajan, J.; Despacio-Reyes, G.; Saunders, G.; Steward, C.; Harte, R.; Lin, M.; Howald, C.; Tanzer, A.; Derrien, T.; Chrast, J.; Walters, N.; Balasubramanian, S.; Pei, B.; Tress, M.; Rodriguez, J. M.; Ezkurdia, I.; van Baren, J.; Brent, M.; Haussler, D.; Kellis, M.; Valencia, A.; Reymond, A.; Gerstein, M.; Guigó, R.; Hubbard, T. J., GENCODE: the reference human genome annotation for The ENCODE Project. Genome Res 2012, 22 (9), 1760-74.
Chen, Y.; Lun, A. T.; Smyth, G. K., From reads to genes to pathways: differential expression analysis of RNA-Seq experiments using Rsubread and the edgeR quasi-likelihood pipeline. F1000Res 2016, 5, 1438.
Tirosh, I.; Izar, B.; Prakadan, S. M.; Wadsworth, M. H.; Treacy, D.; Trombetta, J. J.; Rotem, A.; Rodman, C.; Lian, C.; Murphy, G.; Fallahi-Sichani, M.; Dutton-Regester, K.; Lin, J.-R.; Cohen, O.; Shah, P.; Lu, D.; Genshaft, A. S.; Hughes, T. K.; Ziegler, C. G. K.; Kazer, S. W.; Gaillard, A.; Kolb, K. E.; Villani, A.-C.; Johannessen, C. M.; Andreev, A. Y.; Van Allen, E. M.; Bertagnolli, M.; Sorger, P. K.; Sullivan, R. J.; Flaherty, K. T.; Frederick, D. T.; Jané-Valbuena, J.; Yoon, C. H.; Rozenblatt-Rosen, O.; Shalek, A. K.; Regev, A.; Garraway, L. A., Dissecting the multicellular ecosystem of metastatic melanoma by single-cell RNA-seq. Science 2016, 352 (6282), 189-196.
Dong, B.; Miao, J.; Wang, Y.; Luo, W.; Ji, Z.; Lai, H.; Zhang, M.; Cheng, X.; Wang, J.; Fang, Y.; Zhu, H. H.; Chua, C. W.; Fan, L.; Zhu, Y.; Pan, J.; Wang, J.; Xue, W.; Gao, W.-Q., Single-cell analysis supports a luminal-neuroendocrine transdifferentiation in human prostate cancer. Communications Biology 2020, 3 (1), 778.
Stuart, T.; Butler, A.; Hoffman, P.; Hafemeister, C.; Papalexi, E.; Mauck, W. M., 3rd; Hao, Y.; Stoeckius, M.; Smibert, P.; Satija, R., Comprehensive Integration of Single-Cell Data. Cell 2019, 177 (7), 1888-1902.e21.
Zappia, L.; Oshlack, A., Clustering trees: a visualization for evaluating clusterings at multiple resolutions. GigaScience 2018, 7 (7).
Bunis, D. G.; Andrews, J.; Fragiadakis, G. K.; Burt, T. D.; Sirota, M., dittoSeq: universal user-friendly single-cell and bulk RNA sequencing visualization toolkit. Bioinformatics 2020, 36 (22-23), 5535-5536.

1. A pharmaceutical nucleic acid agent capable of downregulating or inhibiting the activity of the minor spliceosome (MiS) for use in treatment or prevention of recurrence of cancer.

2. The pharmaceutical nucleic acid agent for use according to claim 1, wherein the agent is capable of downregulating or inhibiting expression of snRNA U6atac.

3. The pharmaceutical nucleic acid agent for use according to claim 1, wherein the agent is or encodes an antisense oligonucleotide.

4. The pharmaceutical nucleic acid agent for use according to claim 1, wherein the agent is or encodes an siRNA.

5. The pharmaceutical nucleic acid agent for use according to claim 1, wherein said cancer is a cancer of advanced, therapy resistant phenotype.

6. The pharmaceutical nucleic acid agent for use according to claim 1, wherein said cancer is selected from glioblastoma, breast cancer, chronic myeloid leukemia (CML), bladder cancer, colon cancer, kidney cancer and prostate cancer.

7. The pharmaceutical nucleic acid agent for use according to claim 6, wherein said cancer is prostate cancer.

8. The pharmaceutical nucleic acid agent for use according to claim 7, wherein said cancer is selected from castration-resistant prostate cancer (CRPC), neuroendocrine prostate cancer (NEPC), castration-resistant neuroendocrine prostate cancer (CRPC-NE) and small cell prostate cancer.

9. The pharmaceutical nucleic acid agent for use according to claim 1, wherein said agent is administered in combination with a platinum-containing complex, particularly in combination with a platinum-containing drug selected from carboplatin, satraplatin, cisplatin, dicycloplatin, nedaplatin, oxaliplatin, picoplatin, and/or triplatin.

10. A method for assigning a likelihood of having or developing cancer to a patient, wherein a high likelihood of having or developing cancer is assigned if
an expression level of snRNA U6atac is 2 or 3.

11. The method according to claim 10, wherein a high likelihood of having or developing a cancer of advanced, therapy resistant phenotype is assigned if
an expression level of snRNA U6atac is 2 or 3.

12. The method according to claim 10, wherein said cancer is selected from glioblastoma, breast cancer, chronic myeloid leukemia (CML), bladder cancer, colon cancer, kidney cancer and prostate cancer.

13. The method according to claim 10, wherein said cancer is prostate cancer.

14. The method according to claim 10, wherein said expression level is determined in a tumour sample isolated from said patient.

15. The pharmaceutical nucleic acid agent for use according to claim 1, wherein a high likelihood of having or developing cancer, or of having or developing a cancer of advanced, therapy resistant phenotype is assigned to the patient—if an expression level of snRNA U6atac is 2 or 3.

16. A method for treatment or prevention of recurrence of cancer in a patient, said method comprising administering a pharmaceutical nucleic acid agent according to claim 1 to a patient.

17. A method for treatment or prevention of recurrence of cancer in a patient, said method comprising the steps:
a. obtaining a tumour sample from said patient;
b. determining an expression level of snRNA U6atac in said sample;
c. identifying a patient with high risk of recurrence of cancer if said expression level of snRNA U6atac is 2 or 3;
d. treating said patient with high risk of recurrence of cancer with antineoplastic drug.

18. The method according to claim 17, wherein said patient is treated via administering a pharmaceutical nucleic acid agent capable of downregulating or inhibiting the activity of the minor spliceosome (MiS).

19. A system for performing the method according to claim 10.

20. (canceled)
